(19) United States
(12) Patent Application Publication
(10) Pub. No.: US 2009/0076274 A1
(43) Pub. Date: Mar. 19, 2009

(54) BENZOXAZINONE DERIVATIVES, PREPARATION THEREOF AND USES IN THE TREATMENT OF CNS AND OTHER DISORDERS

(75) Inventors: Barbara Bertani, Verona (IT); Manuela Borriello, Verona (IT); Andrea Bozzoli, Verona (IT); Steven Mark Bromidge, Verona (IT); Enrica Granci, Verona (IT); Colin Leslie, Verona (IT); Halina Serfinowska, Harlow (GB); Luigi Stasi, Verona (IT); Antonio Vong, Harlow (GB); Valeria Zucchelli, Verona (IT)

Correspondence Address: GLAXOSMITHKLINE;Corporate Intellectual Property - UW2220, P.O. Box 1539, King of Prussia, PA, 19406-0939, US

(73) Assignee: Glaxo Group Limited

(21) Appl. No.: 12/269,313

(22) Filed: Nov. 12, 2008

Related U.S. Application Data
(62) Division of application No. 10/535,711, filed on Feb. 7, 2006.

(30) Foreign Application Priority Data
Nov. 21, 2002 (GB) 0227240.9

(51) Int. Cl. C07D 405/02

(52) U.S. Cl. 544/363

(57) ABSTRACT

Intermediates useful in the preparation of compounds of formula (I) and pharmaceutically acceptable salts thereof are disclosed:

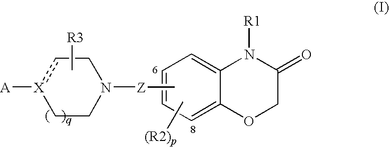

wherein A, R1, R2, R3, [symbol] , p, q, and X are as defined in the specification.

### CROSS REFERENCE TO PRIOR APPLICATIONS

This is a divisional application of U.S. application Ser. No. 10/535,711 filed on Feb. 7, 2006, which is a 371 application of International Application No. PCT/EP2003/013085 filed Nov. 20, 2003, which claims priority to GB 0227240.9 filed Nov. 21, 2002.

The present invention relates to novel compounds, processes for their preparation, pharmaceutical compositions containing the same and their use as medicaments. More particularly this invention relates to novel benzoxazinone derivatives and their utility in the treatment of CNS and other disorders.

WO02/34754 discloses a series of benzoxazinone compounds as being useful for treating certain CNS disorders such as depression. Patent application DT-2429253-A1 discloses certain benzoxazinone compounds including 2H-1,4-benzoxazin-3(4H)-one-6-[[4-(2-naphthalenyl)-1-piperazinyl]acetyl], 2H-1,4-benzoxazin-3(4H)-one-6-[[4-(1-naphthalenyl)-1-piperazinyl]acetyl], 2H-1,4-benzoxazin-3(4H)-one-6-[1-hydroxy-2-[4-(2-naphthalenyl)-1-piperazinyl]ethyl]2H-1,4-benzoxazin-3(4H)-one-6-[1-hydroxy-2-[4-(1-naphthalenyl)-1-piperazinyl]ethyl] and salts thereof, which are claimed to be of sympatholytic, sedative, analgesic and anticholesteremic use.

Artigas (Trends in Pharmacological Sciences, Vol. 14, 262, 1993) suggests that the co-administration of a 5-HT_{1A} receptor antagonist and a selective serotonin reuptake inhibitor (SSRI) may give rise to an improvement in anti-depressant efficacy. Patent applications WO 00/40580 (American Home Products) and WO 00/40581 (American Home Products) both disclose a series of benzoxazine derivatives that are claimed to possess such a combined activity profile.

A novel series of benzoxazinone compounds has now been found that possess high affinity for 5-HT₁ type receptors and/or possess serotonin reuptake inhibition activity. The present invention therefore provides, in a first aspect, a compound of formula (I) or a pharmaceutically acceptable salt thereof:

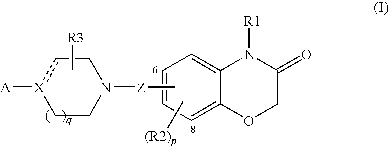

wherein:
A is a bicyclic 6, 5 or 6,6 aromatic or heteroaromatic group which is optionally substituted by 1-4 substituents, which substituents may be the same or different, and which are selected from the group consisting of halogen, hydroxy, cyano, nitro, trifluoromethyl, trifluoromethoxy, C₁₋₆alkyl, trifluoromethanesulfonyloxy, pentafluoroethyl, C₁₋₆alkoxy, arylC₁₋₆alkoxy, C₁₋₆alkylthio, C₁₋₆alkoxyC₁₋₆alkyl, C₃₋₇cycloalkylC₁₋₆alkoxy, C₁₋₆alkanoyl, C₁₋₆alkoxycarbonyl, C₁₋₆alkylsulfonyl, arylsulfonyl, arylsulfonyloxy, C₁₋₆alkylsulfonamido, C₁₋₆alkylamido, arylsulfonamido, arylcarboxamido, aroyl, arylC₁₋₆alkanoyl, and a group Ar¹—B, wherein B represents a single bond, O, S or CH₂ and Ar¹ represents a phenyl or a monocyclic heteroaromatic group, said Ar¹ group being optionally substituted by 1-3 substituents, which may be the same or different, and which are selected from the group consisting of a halogen, hydroxy, cyano, trifluoromethyl, C₁₋₆alkyl, C₁₋₆alkoxy or C₁₋₆alkanoyl;
R1 is hydrogen, C₁₋₆alkyl, haloC₁₋₆alkyl, C₃₋₇cycloalkyl, C₃₋₇cycloalkylC₁₋₆alkyl, C₃₋₆alkenyl, C₃₋₆alkynyl or arylC₁₋₆alkyl;
R2 is independently halogen, C₁₋₆alkyl, cyano, haloC₁₋₆alkyl, C₁₋₆alkanoyl, C₁₋₆alkoxy or hydroxy;
p is 0, 1 or 2;
R3 (a) is a group —(R4)r wherein R4 is selected from the group consisting of: C₁₋₆alkyl, halogen, hydroxy, oxo, cyano, nitro, C₁₋₄alkoxy, haloC₁₋₄alkyl, haloC₁₋₄alkoxy, arylC₁₋₄alkoxy, C₁₋₄alkylthio, hydroxyC₁₋₄alkyl, C₁₋₄alkoxyC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, aroyl, aroylC₁₋₄alkyl, arylC₁₋₄alkanoyl, C₁₋₄acyl, aryl, arylC₁₋₄alkyl, C₁₋₄alkylaminoC₁₋₄alkyl and a group R30R31N- (where each of R30 and R31 independently represents a hydrogen atom or a C₁₋₄alkyl group or where appropriate R30R31 forms part of a C₃₋₆azacyloalkane or C₃₋₆(2-oxo)azacycloalkane ring), and r is 0, 1, 2 or 3; or
(b) forms a bridge across the ring, the bridge consisting of a chain of 1 to 3 atoms, the bridge being optionally substituted by one, two or three groups selected from one, two or three groups selected from halogen, oxo, C₁₋₆alkyl, cyano, haloC₁₋₆alkyl, C₁₋₆alkanoyl, C₁₋₆alkoxy or hydroxy; or
(c) is a chain of 1 to 3 atoms optionally substituted by halogen, C₁₋₆alkyl, cyano, haloC₁₋₆alkyl, C₁₋₆alkanoyl, C₁₋₆alkoxy or hydroxy, the other end of the chain being attached to an available carbon atom in Z;
   X is CH, N or C; [symbol] represents a single bond when X is CH or N; and [symbol] represents a double bond when X is C;
   q is 0, 1 or 2, wherein when q is 0, X is not N;
   Z is attached to the 6-position or the 8-position of the benzoxazinone group and is a 3 to 7 membered cycloalkylene group, 3 to 7 membered cycloalkenylene group, —(CH═CH)— or a group

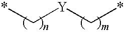

wherein m and n are independently 0, 1 or 2, and Y is a single bond, 3 to 7 membered cycloalkylene group, 3 to 7 membered cycloalkenylene group, —(CH═CH)—, —C(═O)—, —C(═CH₂)—, oxygen, or a methylene group optionally substituted by one or two groups selected from halogen, C₁₋₆alkyl, cyano, haloC₁₋₆alkyl, C₁₋₆alkanoyl, C₁₋₆alkoxy or hydroxy;
provided that when A is naphthyl, 5,6,7,8-tetrahydronaphthyl or 2,3-dihydroindene, Z is not —(CH₂CH(OH))—, —(CH₂CH₂CH(OH))— or —(CH₂C(═O).

Where used herein the term naphthyl, whether alone or as part of another group, is intended, unless otherwise stated, to denote both 1-naphthyl and 2-naphthyl groups.

The term “bicyclic 6, 5 or 6,6 aromatic or heteroaromatic group” refers to a stable bicyclic aromatic group having 9 or 10 carbon atoms in total, as well as a stable bicyclic heteroaromatic group having 9 or 10 atoms in total and containing 1 to 4 heteroatoms selected from oxygen, nitrogen and sulfur; in either case, both the rings in the bicyclic group may be unsaturated, or one of the two rings may be saturated or partially saturated. Examples of bicyclic 6, 5 or 6,6 aromatic groups in which both the rings are unsaturated include naphthyl and indene; examples of bicyclic 6, 5 or 6,6 aromatic groups in which one of the two rings is saturated or partially saturated include 5,6,7,8-tetrahydronaphthyl and 2,3-dihydroindene. Examples of bicyclic 6, 5 or 6,6 heteroaromatic groups in which both the rings are unsaturated include indolyl, quinolyl, quinazolinyl, isoquinolyl, benzofuranyl, benzothienyl, benzimidazolyl, indazolyl, 4-, 5-, 6- or 7-azaindolyl, benzothiazolyl, benzoxazolyl, benzisoxazolyl, benzisothiazolyl, quinoxalinyl and cinnolinyl. Examples of bicyclic 6, 5 or 6,6 heteroaromatic groups in which one of the two rings is saturated or partially saturated include 2,3-dihydrobenzodioxinyl.

The term “monocyclic heteroaromatic group” refers to stable monocyclic heteroaromatic groups having 5 or 6 atoms in total and containing 1 to 4 heteroatoms selected from oxygen, nitrogen and sulfur. Examples of monocyclic heteroaromatic groups include pyrrolyl, pyrrolinyl, pyrazolinyl, imidazolyl, pyrazolyl, oxadiazolyl, isothiazolyl, thiazolyl, thiazinyl, furyl, thienyl, pyridyl, pyridazinyl, pyrimidinyl and pyrazinyl.

The term “aryl”, whether alone or as part of another group, is intended, unless otherwise stated, to denote an aromatic carbocyclic or heterocyclic group such as phenyl, pyrrolyl, pyrrolinyl, pyrazolinyl, imidazolyl, pyrazolyl, oxadiazolyl, isothiazolyl, thiazolyl, thiazinyl, furyl, thienyl, pyridyl, pyridazinyl, pyrimidinyl, pyrazinyl, azepinyl or naphthyl, optionally substituted by one or more halogen, C₁₋₆alkyl, CF₃, cyano, hydroxy, C₁₋₆alkanoyl, or C₁₋₆alkoxy.

The term “C₁₋₆alkyl”, whether alone or part of another group, refers to alkyl groups having from one to six carbon atoms, in all isomeric forms, including methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “halogen” is used herein to describe, unless otherwise stated, a group selected from fluorine, chlorine, bromine and iodine.

The term “haloC₁₋₆alkyl” refers to C₁₋₆alkyl groups with one or more halo substituents, for example CF₃.

The term “C₁₋₆alkanoyl” refers to an alkanoyl group having from 1 to 6 carbon atoms, such as methanoyl (or “formyl”), ethanoyl (or “acetyl”), propanoyl, butanoyl, pentanoyl and hexanoyl.

The term “C₁₋₆alkoxy” refers to a straight chain or branched chain alkoxy (or “alkyloxy”) group having from one to six carbon atoms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “3 to 7 membered cycloalkylene group” refers to cycloalkylene groups having from 3 to 7 carbons, such as cyclohexylene.

The term “3 to 7 membered cycloalkenylene group” refers to cycloalkenylene groups having from 3 to 7 carbons, such as cyclohexenylene.

The term “C₁₋₆alkylthio” refers to a straight chain or branched chain alkylthio group having from one to six carbon atoms, such as methylthio, ethylthio, propylthio, isopropylthio, butylthio, isobutylthio, sec-butylthio, tert-butylthio, pentylthio, neopentylthio, sec-pentylthio, n-pentylthio, isopentylthio, tert-pentylthio and hexylthio.

The term “arylC₁₋₆alkoxy” refers to an aryl group which is linked by a C₁₋₆alkoxy group. Examples include phenylmethoxy, phenylethoxy, naphthymethoxy, naphthylethoxy, phenylpropoxy, naphthylpropoxy, phenylbutoxy and naphthylpentoxy.

The term “C₃₋₇cycloalkyl” refers to a cycloalkyl group consisting of from 3 to 7 carbon atoms, for example cyclopropane, cyclobutane, cyclopentane, cyclohexane and cycloheptane.

The term “aroyl” refers to a group having the formula “aryl-CO” wherein “aryl” is as defined above.

The term “C₃₋₆alkenyl” refers to an unsaturated hydrocarbon group containing one or more C═C bonds and having from three to six carbon atoms, in all isomeric forms, such as propenyl, butenyl, pentenyl, and hexenyl.

The term “C₃₋₆alkynyl” refers to an unsaturated hydrocarbon group containing one or more triple C—C bonds, having from three to six carbon atoms, in all isomeric forms, such as propynyl, butylidyne, pentenynyl, and pentylidyne.

When R1 is C₁₋₆alkyl, a preferred group is methyl or ethyl. Preferably R1 is hydrogen or methyl.

When p is other than 0, preferably R2 is halogen (particularly fluoro or chloro) or C₁₋₆alkyl (particularly methyl or ethyl).

R3 may be a group —(R4)r wherein R4 is selected from the group consisting of: C₁₋₆alkyl, halogen, hydroxy, oxo, cyano, nitro, C₁₋₄alkoxy, haloC₁₋₄alkyl, haloC₁₋₄alkoxy, arylC₁₋₄alkoxy, C₁₋₄alkylthio, hydroxyC₁₋₄alkyl, C₁₋₄alkoxyC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, aroyl, aroylC₁₋₄alkyl, arylC₁₋₄alkanoyl, C₁₋₄acyl, aryl, arylC₁₋₄alkyl, C₁₋₄alkylaminoC₁₋₄alkyl and a group R30R31N- (where each of R30 and R31 independently represents a hydrogen atom or a C₁₋₄alkyl group or where appropriate R30R31 forms part of a C₃₋₆azacyloalkane or C₃₋₆(2-oxo)azacycloalkane ring). If R3 is a group —(R4)r, r may be 0, 1, 2 or 3. If r is 2 or 3, the 2 or 3 R4 groups are independently selected from the above group.

Preferably R3 is a group —(R4)r. Preferably R4 is methyl and r is 0 or 1.

R3 may alternatively form a bridge across the ring to which it is attached, wherein the bridge consists of a chain of 1 to 3 atoms, the bridge being optionally substituted by one, two or three groups selected from halogen, oxo, C₁₋₆alkyl, cyano, haloC₁₋₆alkyl, C₁₋₆alkanoyl, C₁₋₆alkoxy or hydroxy. Suitably, the chain of atoms consists of 1 to 3 atoms selected from carbon, oxygen, nitrogen and sulfur. Examples of groups formed when R3 is a chain of 1 to 3 atoms forming a bridge across the ring are:

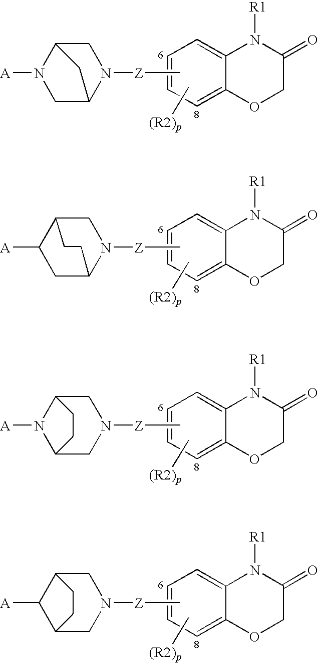

R3 may alternatively be a chain of 1 to 3 atoms optionally substituted by halogen, C₁₋₆alkyl, cyano, haloC₁₋₆alkyl, C₁₋₆alkanoyl, C₁₋₆alkoxy or hydroxy, wherein the chain is attached to an available carbon atom in group Z. Suitably, the chain of atoms consists of 1 to 3 atoms selected from carbon, oxygen, nitrogen and sulfur. Examples of compounds wherein R3 forms a chain of atoms attached to an available carbon atom in group Z include:

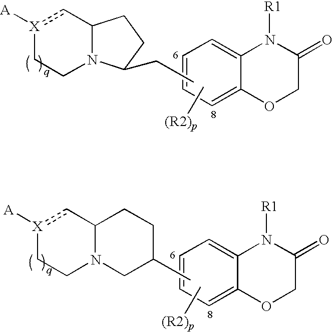

Preferably X is CH or N and [symbol] is a single bond.

Preferably q is 1.

Preferably Z is —(CH₂)₂— or —(CH₂)₃—.

If A is a bicyclic 6, 5 or 6,6 aromatic group, preferably A is 5,6,7,8-tetrahydronapthalenyl, optionally substituted by 1-4 substituents, which substituents may be the same or different, and which are selected from the group consisting of halogen, hydroxy, cyano, nitro, trifluoromethyl, trifluoromethoxy, C₁₋₆alkyl, trifluoromethanesulfonyloxy, pentafluoroethyl, C₁₋₆alkoxy, arylC₁₋₆alkoxy, C₁₋₆alkylthio, C₁₋₆alkoxyC₁₋₆alkyl, C₃₋₇cycloalkylC₁₋₆alkoxy, C₁₋₆alkanoyl, C₁₋₆alkoxycarbonyl, C₁₋₆alkylsulfonyl, arylsulfonyl, arylsulfonyloxy, C₁₋₆alkylsulfonamido, C₁₋₆alkylamido, arylsulfonamido, arylcarboxamido, aroyl, arylC₁₋₆alkanoyl, and a group Ar¹—B, wherein B represents a single bond, O, S or CH₂ and Ar¹ represents a phenyl or a monocyclic heteroaromatic group, said Ar¹ group being optionally substituted by 1-3 substituents, which may be the same or different, and which are selected from the group consisting of a halogen, hydroxy, cyano, trifluoromethyl, C₁₋₆alkyl, C₁₋₆alkoxy or C₁₋₆alkanoyl. Preferably A is a bicyclic 6, 5 or 6,6 heteroaromatic group which is optionally substituted by 1-4 substituents as defined above.

Preferably A is indolyl, quinolyl, quinazolinyl or 2,3-dihydrobenzodioxinyl, said groups being optionally substituted by 1-4 substituents, which substituents may be the same or different, and which are selected from the group consisting of halogen, hydroxy, cyano, nitro, trifluoromethyl, trifluoromethoxy, C₁₋₆alkyl, trifluoromethanesulfonyloxy, pentafluoroethyl, C₁₋₆alkoxy, arylC₁₋₆alkoxy, C₁₋₆alkylthio, C₁₋₆alkoxyC₁₋₆alkyl, C₃₋₇cycloalkylC₁₋₆alkoxy, C₁₋₆alkanoyl, C₁₋₆alkoxycarbonyl, C₁₋₆alkylsulfonyl, arylsulfonyl, arylsulfonyloxy, C₁₋₆alkylsulfonamido, C₁₋₆alkylamido, arylsulfonamido, arylcarboxamido, aroyl, arylC₁₋₆alkanoyl, and a group Ar¹—B, wherein B represents a single bond, O, S or CH₂ and Ar¹ represents a phenyl or a monocyclic heteroaromatic group, said Ar¹ group being optionally substituted by 1-3 substituents, which may be the same or different, and which are selected from the group consisting of a halogen, hydroxy, cyano, trifluoromethyl, C₁₋₆alkyl, C₁₋₆alkoxy or C₁₋₆alkanoyl.

When a substituent on A is a further group Ar¹—B, Ar¹ is preferably a monocyclic heteroaromatic group (particularly isoxazolyl or oxadiazolyl), optionally substituted as defined above. Preferably B is a single bond.

Preferred optional substituents for A are halogen (particularly fluoro or chloro), C₁₋₆alkyl (particularly methyl, ethyl and propyl), cyano, CF₃, C₁₋₆alkoxy (particularly methoxy, ethoxy or isopropoxy), C₁₋₆alkanoyl or a group Ar¹—B as defined above.

Most particularly preferred A groups, including optional substituents, are 5-quinolyl(2-Me), 5-quinolyl(2-Me, 7-Cl), 5-quinolyl(2-Me, 7-F) and 5-quinazolinyl(2-Me), 5-quinolyl(2-Me, 7-Me), 5-dihydrobenzo[1,4]dioxinyl, 8-quinolyl(6-methoxy), 8-quinolyl, 4-indolyl and 4-indolyl (2-Me).

In a further aspect the present invention provides a compound of formula (Ia) or a pharmaceutically acceptable salt thereof:

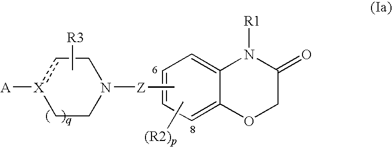

wherein:
A is a bicyclic 6, 5 or 6,6 heteroaromatic group which is optionally substituted by 1-4 substituents, which substituents may be the same or different, and which are selected from the group consisting of halogen, hydroxy, cyano, nitro, trifluoromethyl, trifluoromethoxy, C₁₋₆alkyl, trifluoromethanesulfonyloxy, pentafluoroethyl, C₁₋₆alkoxy, arylC₁₋₆alkoxy, C₁₋₆alkylthio, C₁₋₆alkoxyC₁₋₆alkyl, C₃₋₇cycloalkylC₁₋₆alkoxy, C₁₋₆alkanoyl, C₁₋₆alkoxycarbonyl, C₁₋₆alkylsulfonyl, arylsulfonyl, arylsulfonyloxy, C₁₋₆alkylsulfonamido, C₁₋₆alkylamido, arylsulfonamido, arylcarboxamido, aroyl, arylC₁₋₆alkanoyl, and a group Ar¹—B, wherein B represents a single bond, O, S or CH₂ and Ar¹ represents a phenyl or a monocyclic heteroaromatic group, said Ar¹ group being optionally substituted by 1-3 substituents, which may be the same or different, and which are selected from the group consisting of a halogen, hydroxy, cyano, trifluoromethyl, C₁₋₆alkyl, C₁₋₆alkoxy or C₁₋₆alkanoyl;
R1 is hydrogen, C₁₋₆alkyl, haloC₁₋₆alkyl, C₃₋₇cycloalkyl, C₃₋₇cycloalkylC₁₋₆alkyl, C₃₋₆alkenyl, C₃₋₆alkynyl or arylC₁₋₆alkyl;
R2 is independently halogen, C₁₋₆alkyl, cyano, haloC₁₋₆alkyl, C₁₋₆alkanoyl, C₁₋₆alkoxy or hydroxy;
p is 0, 1 or 2;
R3 (a) is a group —(R4)r wherein R4 is selected from the group consisting of: C₁₋₆alkyl, halogen, hydroxy, oxo, cyano, nitro, C₁₋₄alkoxy, haloC₁₋₄alkyl, haloC₁₋₄alkoxy, arylC₁₋₄alkoxy, C₁₋₄alkylthio, hydroxyC₁₋₄alkyl, C₁₋₄alkoxyC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, aroyl, aroylC₁₋₄alkyl, arylC₁₋₄alkanoyl, C₁₋₄acyl, aryl, arylC₁₋₄alkyl, C₁₋₄alkylaminoC₁₋₄alkyl and a group R30R31N- (where each of R30 and R31 independently represents a hydrogen atom or a C₁₋₄alkyl group or where appropriate R30R31 forms part of a C₃₋₆azacyloalkane or C₃₋₆(2-oxo)azacycloalkane ring), and r is 0, 1, 2 or 3; or
(b) forms a bridge across the ring, the bridge consisting of a chain of 1 to 3 atoms, the bridge being optionally substituted by one, two or three groups selected from halogen, oxo, C₁₋₆alkyl, cyano, haloC₁₋₆alkyl, C₁₋₆alkanoyl, C₁₋₆alkoxy or hydroxy; or
(c) is a chain of 1 to 3 atoms optionally substituted by halogen, C₁₋₆alkyl, cyano, haloC₁₋₆alkyl, C₁₋₆alkanoyl, C₁₋₆alkoxy or hydroxy, the other end of the chain being attached to an available carbon atom in Z;

### X is CH, N or C;

[symbol] represents a single bond when X is CH or N; and [symbol] represents a double bond when X is C;
   q is 0, 1 or 2, wherein when q is 0, X is not N;
   Z is attached to the 6-position or the 8-position of the benzoxazinone group and is a 3 to 7 membered cycloalkylene group, 3 to 7 membered cycloalkenylene group, —(CH═CH)— or a group

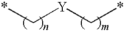

wherein m and n are independently 0, 1 or 2, and Y is a single bond, 3 to 7 membered cycloalkylene group, 3 to 7 membered cycloalkenylene group, —(CH═CH)—, —C(═O)—, —C(═CH₂)—, oxygen, or a methylene group optionally substituted by one or two groups selected from halogen, C₁₋₆alkyl, cyano, haloC₁₋₆alkyl, C₁₋₆alkanoyl, C₁₋₆alkoxy or hydroxy.

Preferred features of formula (I) apply to formula (Ia) mutatis mutandis.

Preferred compounds of this invention are:
6-{2-[4-(2-Methylquinolin-5-yl)piperazin-1-yl]ethyl}-4H-benzo[1.4]oxazin-3-one
6-{2-[4-(2,7-Dimethylquinolin-5-yl)piperazin-1-yl]ethyl}-4H-benzo[1,4]oxazin-3-one
6-{2-[4-(7-Chloro-2-methylquinolin-5-yl)piperazin-1-yl]ethyl}-4H-benzo[1,4]oxazin-3-one
6-[2-(4-Quinolin-4-ylpiperazin-1-yl)ethyl]-4H-benzo[1.4]oxazin-3-one
6-{2-[4-(2-Methylquinazolin-5-yl)piperazin-1-yl]ethyl}-4H-benzo[1.4]oxazin-3-one
6-{2-[4-(2,3-Dihydrobenzo[1,4]dioxin-5-yl)piperazin-1-yl]ethyl}-4H-benzo[1.4]oxazin-3-one
6-{2-[4-(6-Methoxyquinolin-8-yl)piperazin-1-yl]ethyl}-4H-benzo[1,4]oxazin-3-one
6-[2-(4-Quinolin-8-yl)piperazin-1-yl)ethyl]-4H-benzo[1,4]oxazin-3-one
6-{2-[4-(1H-Indol-4-yl)piperazin-1-yl]ethyl}-4H-benzo[1,4]oxazin-3-one
6-{2-[4-(7-Chloro-2-methylquinolin-5-yl)piperazin-1-yl]ethyl}-7-fluoro-4H-benzo[1,4]oxazin-3-one
4-Methyl-6-{2-[4-(2-methylquinolin-5-yl)piperazin-1-yl]ethyl}-4H-benzo[1,4]oxazin-3-one
6-{2-[4-(2-Methylquinolin-5-yl)piperazin-1-yl]ethanoyl}-4H-benzo[1,4]oxazin-3-one
6-{1-Hydroxy-2-[4-(2-methylquinolin-5-yl)piperazin-1-yl]ethyl}-4H-benzo[1,4]oxazin-3-one
6-{2-[4-(2-Methyl-4-(2-methylquinolin-5-yl)piperazin-1-yl]ethyl}-4H-benzo[1,4]oxazin-3-one
6-{2-[3-Methyl-4-(2-methylquinolin-5-yl)piperazin-1-yl]ethyl}-4H-benzo[1,4]oxazin-3-one
6-{2-[2-Methyl-4-(2-methylquinolin-5-yl)piperazin-1-yl]ethyl}-4H-benzo[1,4]oxazin-3-one
6-{2-[4-(2-Methylquinolin-5-yl)-3,6-dihydro-2H-pyridin-1-yl]ethyl}-4H-benzo[1,4]oxazin-3-one
6-{2-[4-(2-Methylquinolin-5-yl)piperidin-1-yl]ethyl}-4-H-benzo[1,4]oxazin-3-one
6-{2-[4-(2-Methylquinolin-5-yl)-[1,4]diazepan-1-yl]ethyl}-4H-benzo[1,4]oxazin-3-one
6-{2-[4-(2-Methylquinazolin-5-yl)-[1,4]diazepan-1-yl]ethyl}-4H-benzo[1,4]oxazin-3-one
7-Fluoro-6-{2-[4-(2-methylquinolin-5-yl)piperazin-1-yl]ethyl}-4H-benzo[1,4]oxazin-3-one
6-{3-[4-(2-Methylquinolin-5-yl)-piperazin-1-yl]-propyl}-4H-benzo[1,4]-oxa-zin-3-one
6-{3-[4-(7-Fluoro-2-methylquinolin-5-yl)piperazin-1-yl]-propyl}-4H-benzo-[1,4]oxazin-3-one
6-{3-[4-(2-Methylquinolin-5-yl)-piperazin-1-yl]-propanoyl}-4H-benzo[1,4]-oxa-zin-3-one
6-{1-Hydroxy-3-[4-(2-methylquinolin-5-yl)-piperazin-1-yl]-propyl}-4H-benzo-[1,4]oxazin-3-one
6-{(E)-3-[4-(2-Methylquinolin-5-yl)piperazin-1-yl]propenyl}-4H-benzo[1,4]-oxa-zin-3-one
6-{4-[4-(2-Methylquinolin-5-yl)piperazin-1-yl]butyl}-4H-benzo[1,4]oxazin-3-one
6-{4-[4-(2-Methylquinolin-5-yl)piperazin-1-yl]-cyclohex-1-enyl}-4H-benzo[1,4]-oxazin-3-one
6-{4-[4-(2-Methylquinazolin-5-yl)piperazin-1-yl]butyl}-4H-benzo[1,4]oxazin-3-one
6-{2-[4-(2-Methylquinolin-5-yl)piperazin-1-yl]ethoxy}-4-H-benzo[1,4]oxazin-3-one
4-Methyl-6-{2-[4-(2-methylquinolin-5-yl)piperazin-1-yl]ethoxy}-4-H-benzo[1,4]oxazin-3-one
7-Fluoro-6-{2-[4-(7-fluoro-2-methylquinolin-5-yl)piperazin-1-yl]ethyl}-4H-benzo[1,4]oxazin-3-one
6-{2-[4-(7-fluoro-2-methylquinolin-5-yl)piperazin-1-yl]ethyl}-4H-benzo[1,4]oxazin-3-one
7-Fluoro-6-{2-[4-(2-methylquinolin-5-yl)piperazin-1-yl]ethanoyl}-4H-benzo[1,4]oxazin-3-one
6-{1-Hydroxy-2-[4-(2-methylquinolin-5-yl)piperazin-1-yl]ethyl}-4H-benzo[1,4]-oxazin-3-one
6-{1-Methoxy-3-[4-(2-methylquinolin-5-yl)piperazin-1-yl]propyl}-4H-benzo[1,4]-oxazin-3-one
6-{2-[4-(2-Methyl-1H-indol-4-yl)piperazin-1-yl]-ethyl}-4H-benzo-[1,4]oxazin-3-one
6-{2-[4-(5,6,7,8-Tetrahydronaphthalen-1-yl)piperazin-1-yl]ethyl}-4H-benzo-[1,4]oxazin-3-one
6-[2-(4-Naphthalen-1-ylpiperazin-1-yl)ethyl]-4H-benzo[1,4]oxazin-3-one hydrochloride salt
6-{1-Fluoro-2-[4-(2-methylquinolin-5-yl)piperazin-1-yl]ethyl}-4H-benzo[1,4]-oxazin-3-one
6-{1-Fluoro-3-[4-(2-methylquinolin-5-yl)piperazin-1-yl]propyl}-4H-benzo[1,4]-oxazin-3-one
5-Fluoro-6-{2-[4-(2-methylquinolin-5-yl)piperazin-1-yl]ethyl}-4H-benzo[1,4]-oxazin-3-one
5-Fluoro-4-methyl-6-{2-[4-(2-methylquinolin-5-yl)piperazin-1-yl]ethyl}-4H-benzo[1,4]-oxazin-3-one
6-{2-[4-(7-Chloro-2-methylquinolin-5-yl)piperazin-1-yl]ethyl}-4-methyl-4H-benzo-[1,4]-oxazin-3-one
4-Ethyl-6-{2-[4-(2-methylquinolin-5-yl)piperazin-1-yl]ethyl}-4H-benzo[1,4]-oxazin-3-one
6-{2-[4-(7-Fluoro-2-methylquinolin-5-yl)piperazin-1-yl]ethyl}-4-methyl-4H-benzo-[1,4]-oxazin-3-one
6-{1-(Methyloxy)-2-[4-(2-methyl-5-quinolinyl)-1-piperazinyl]ethyl}-2H-1,4-benzoxazin-3(4H)-one
6-{1-Amino-2-[4-(2-methyl-5-quinolinyl)-1-piperazinyl]ethyl}-2H-1,4-benzoxazin-3(4H)-one
N-[2-[4-(2-Methyl-5-quinolinyl)-1-piperazinyl]-1-(3-oxo-3,4-dihydro-2H-1,4-benzoxazin-6-yl)ethyl]acetamide
6-{1-(Methylamino)-2-[4-(2-methyl-5-quinolinyl)-1-piperazinyl]ethyl}-2H-1,4-benzoxazin-3(4H)-one
6-[2-[4-(2-Methyl-5-quinolinyl)-1-piperazinyl]-1-(phenyloxy)ethyl]-2H-1,4-benzoxazin-3(4H)-one
[2-[4-(2-Methyl-5-quinolinyl)-1-piperazinyl]-1-(3-oxo-3,4-dihydro-2H-1,4-benzoxazin-6-yl)ethyl]formamide
6-{1-Hydroxy-1-methyl-3-[4-(2-methyl-5-quinolinyl)-1-piperazinyl]propyl}-2H-1,4-benzoxazin-3(4H)-one
6-{1-Hydroxy-1-methyl-2-[4-(2-methyl-5-quinolinyl)-1-piperazinyl]ethyl}-2H-1,4-benzoxazin-3(4H)-one
6-{(1E)-1-Methyl-3-[4-(2-methyl-5-quinolinyl)-1-piperazinyl]-1-propen-1-yl}-2H-1,4-benzoxazin-3(4H)-one
6-(1-{2-[4-(2-Methyl-5-quinolinyl)-1-piperazinyl]ethyl}ethenyl)-2H-1,4-benzoxazin-3(4H)-one
6-(1-{[4-(2-Methyl-5-quinolinyl)-1-piperazinyl]methyl}ethenyl)-2H-1,4-benzoxazin-3(4H)-one
2-[4-(2-Methyl-5-quinolinyl)-1-piperazinyl]-1-(3-oxo-3,4-dihydro-2H-1,4-benzoxazin-6-yl)ethyl acetate
6-{1-Hydroxy-2-[4-(2-methyl-5-quinolinyl)-1-piperazinyl]ethyl}-2H-1,4-benzoxazin-3(4H)-one
6-{[4-(8-Quinolinyl)-1-piperazinyl]methyl}-2H-1,4-benzoxazin-3(4H)-one
6-{2-[(1S,4S)-5-(2-Methyl-5-quinolinyl)-2,5-diazabicyclo[2.2.1]hept-2-yl]ethyl}-2H-1,4-benzoxazin-3(4H)-one
6-{2-[4-(2-Quinolinyl)-1-piperazinyl]ethyl}-2H-1,4-benzoxazin-3(4H)-one
6-{3-[4-(2-Quinolinyl)-1-piperazinyl]propyl}-2H-1,4-benzoxazin-3(4H)-one
6-{2-[4-(6-Chloro-2-quinolinyl)-1-piperazinyl]ethyl}-2H-1,4-benzoxazin-3(4H)-one
6-{2-[4-(6-Nitro-2-quinolinyl)-1-piperazinyl]ethyl}-2H-1,4-benzoxazin-3(4H)-one
6-{2-[4-(7-Methyl-1,8-naphthyridin-4-yl)-1-piperazinyl]ethyl}-2H-1,4-benzoxazin-3(4H)-one
6-{2-[4-(1,6-Naphthyridin-5-yl)-1-piperazinyl]ethyl}-2H-1,4-benzoxazin-3(4H)-one
6-{2-[4-(2-Phenylquinolin-5-yl)piperazin-1-yl]ethyl}-4H-benzo[1,4]oxazin-3-one
6-{[4-(7-Fluoro-2-methyl-5-quinolinyl)-1-piperazinyl]acetyl}-2H-1,4-benzoxazin-3(4H)-one
6-{2-[4-(7-Fluoro-2-methyl-5-quinolinyl)-1-piperazinyl]-1-hydroxyethyl}-2H-1,4-benzoxazin-3(4H)-one
6-{1-Fluoro-2-[4-(7-fluoro-2-methyl-5-quinolinyl)-1-piperazinyl]ethyl}-2H-1,4-benzoxazin-3(4H)-one
8-Fluoro-6-{2-[4-(2-methyl-5-quinolinyl)-1-piperazinyl]ethyl}-2H-1,4-benzoxazin-3(4H)-one
8-Fluoro-6-{[4-(2-methyl-5-quinolinyl)-1-piperazinyl]acetyl}-2H-1,4-benzoxazin-3(4H)-one
8-Fluoro-6-{1-hydroxy-2-[4-(2-methyl-5-quinolinyl)-1-piperazinyl]ethyl}-2H-1,4-benzoxazin-3(4H)-one
8-Fluoro-6-{1-fluoro-2-[4-(2-methyl-5-quinolinyl)-1-piperazinyl]ethyl}-2H-1,4-benzoxazin-3(4H)-one
8-Fluoro-6-{2-[4-(7-fluoro-2-methyl-5-quinolinyl)-1-piperazinyl]ethyl}-2H-1,4-benzoxazin-3(4H)-one
8-Fluoro-6-{[4-(7-fluoro-2-methyl-5-quinolinyl)-1-piperazinyl]acetyl}-2H-1,4-benzoxazin-3(4H)-one
8-Fluoro-6-{2-[4-(7-fluoro-2-methyl-5-quinolinyl)-1-piperazinyl]-1-hydroxyethyl}-2H-1,4-benzoxazin-3(4H)-one
6-{[4-(8-Chloro-2-methyl-5-quinolinyl)-1-piperazinyl]acetyl}-2H-1,4-benzoxazin-3(4H)-one
6-{2-[4-(8-Chloro-2-methyl-5-quinolinyl)-1-piperazinyl]-1-hydroxyethyl}-2H-1,4-benzoxazin-3(4H)-one
6-{2-[4-(8-Chloro-2-methyl-5-quinolinyl)-1-piperazinyl]ethyl}-2H-1,4-benzoxazin-3(4H)-one
4-Methyl-8-{2-[4-(2-methyl-5-quinolinyl)-1-piperazinyl]ethyl}-2H-1,4-benzoxazin-3(4H)-one
8-{2-[4-(2-Methyl-5-quinolinyl)-1-piperazinyl]ethyl}-2H-1,4-benzoxazin-3(4H)-one
6-{2-[4-(7-Chloro-2-methyl-5-quinolinyl)-1-piperazinyl]ethyl}-7-fluoro-2H-1,4-benzoxazin-3(4H)-one
6-{2-[(2S)-2-Methyl-4-(2-methyl-5-quinolinyl)-1-piperazinyl]ethyl}-2H-1,4-benzoxazin-3(4H)-one
6-{2-[(2R)-2-Methyl-4-(2-methyl-5-quinolinyl)-1-piperazinyl]ethyl}-2H-1,4-benzoxazin-3(4H)-one
6-{2-[4-(2,3-dihydro-1,4-benzodioxin-6-yl)-1-piperazinyl]ethyl}-2H-1,4-benzoxazin-3(4H)-one
6-{2-[4-(3,4-dihydro-2H-1,5-benzodioxepin-7-yl)-1-piperazinyl]ethyl}-2H-1,4-benzoxazin-3(4H)-one
6-{2-[4-(7-bromo-1H-indol-4-yl)-1-piperazinyl]ethyl}-2H-1,4-benzoxazin-3(4H)-one
6-{3-[4-(7-bromo-1H-indol-4-yl)-1-piperazinyl]propyl}-2H-1,4-benzoxazin-3(4H)-one
6-{2-[4-(1-isoquinolinyl)-1-piperazinyl]ethyl}-2H-1,4-benzoxazin-3(4H)-one
ethyl 5-{4-[2-(3-oxo-3,4-dihydro-2H-1,4-benzoxazin-6-yl)ethyl]-1-piperazinyl}-1-benzofuran-2-carboxylate
6-{2-[4-(1,2-dihydro-5-acenaphthylenyl)-1-piperazinyl]ethyl}-2H-1,4-benzoxazin-3(4H)-one
6-{2-[4-(5-fluoro-1H-indol-3-yl)-1-piperidinyl]ethyl}-2H-1,4-benzoxazin-3(4H)-one
6-{2-[4-(5-chloro-1H-indol-4-yl)-1-piperazinyl]ethyl}-2H-1,4-benzoxazin-3(4H)-one
6-{2-[4-(6-chloro-1H-indol-4-yl)-1-piperazinyl]ethyl}-2H-1,4-benzoxazin-3(4H)-one
6-{2-[4-(1H-pyrrolo[2,3-b]pyridin-4-yl)-1-piperazinyl]ethyl}-2H-1,4-benzoxazin-3(4H)-one
6-{2-[4-(7-chloro-1H-indol-4-yl)-1-piperazinyl]ethyl}-2H-1,4-benzoxazin-3(4H)-one
6-{3-[4-(1H-pyrrolo[2,3-b]pyridin-4-yl)-1-piperazinyl]propyl}-2H-1,4-benzoxazin-3(4H)-one
6-{3-[4-(5-chloro-1H-indol-4-yl)-1-piperazinyl]propyl}-2H-1,4-benzoxazin-3(4H)-one
6-{2-[4-(5-methylthieno[2,3-d]pyrimidin-4-yl)-1-piperazinyl]ethyl}-2H-1,4-benzoxazin-3(4H)-one
6-({2-[4-(2-methyl-5-quinazolinyl)-1-piperazinyl]ethyl}oxy)-2H-1,4-benzoxazin-3(4H)-one
6-{2-[4-(7-Chloro-2-methylquinolin-5-yl)piperazin-1-yl]ethanoyl}-4H-benzo[1.4]oxazin-3-one
6-{2-[4-(7-Chloro-2-methyl-5-quinolinyl)-1-piperazinyl]-1-hydroxyethyl}-2H-1,4-benzoxazin-3(4H)-one
6-{2-[4-(7-Chloro-2-methyl-5-quinolinyl)-1-piperazinyl]-1-fluoroethyl}-2H-1,4-benzoxazin-3(4H)-one
6-{3-[4-(2,2-Dimethyl-2,3-dihydro-1-benzofuran-7-yl)-1-piperazinyl]propyl}-2H-1,4-benzoxazin-3(4H)-one
6-{2-[4-(2,2-dimethyl-2,3-dihydro-1-benzofuran-7-yl)-1-piperazinyl]ethyl}-2H-1,4-benzoxazin-3(4H)-one
4-Methyl-6-{[4-(1H-pyrrolo[2,3-b]pyridin-3-yl)-3,6-dihydro-1(2H)-pyridinyl]acetyl}-2H-1,4-benzoxazin-3(4H)-one
6-{1-hydroxy-2-[4-(1H-pyrrolo[2,3-b]pyridin-3-yl)-3,6-dihydro-1(2H)-pyridinyl]ethyl}-4-methyl-2H-1,4-benzoxazin-3(4H)-one
6-{[4-(2-Methyl-5-quinolinyl)-1-piperazinyl]methyl}-2H-1,4-benzoxazin-3(4H)-one
4-methyl-6-{[4-(2-methyl-5-quinolinyl)-1-piperazinyl]acetyl}-3,4-dihydro-2H-1,4-benzoxazin-2-one
4-Methyl-6-(1-{[4-(2-methyl-5-quinolinyl)-1-piperazinyl]methyl}ethenyl)-3,4-dihydro-2H-1,4-benzoxazin-2-one
6-(2-Hydroxy-1-{[4-(2-methyl-5-quinolinyl)-1-piperazinyl]methyl}ethyl)-4-methyl-3,4-dihydro-2H-1,4-benzoxazin-2-one
6-{[4-(6-fluoro-2-methyl-5-quinolinyl)-1-piperazinyl]acetyl}-2H-1,4-benzoxazin-3(4H)-one
6-{2-[4-(6-Fluoro-2-methyl-5-quinolinyl)-1-piperazinyl]-1-hydroxyethyl}-2H-1,4-benzoxazin-3(4H)-one
6-{1-Hydroxy-2-[4-(2-methyl-5-quinolinyl)-1-piperazinyl]ethyl}-4-methyl-2H-1,4-benzoxazin-3(4H)-one
6-{[4-(8-Fluoro-2-methyl-5-quinolinyl)-1-piperazinyl]acetyl}-4-methyl-2H-1,4-benzoxazin-3(4H)-one
6-{2-[4-(8-Fluoro-2-methyl-5-quinolinyl)-1-piperazinyl]-1-hydroxyethyl}-4-methyl-2H-1,4-benzoxazin-3(4H)-one
6-{[4-(6-Fluoro-2-methyl-5-quinolinyl)-1-piperazinyl]acetyl}-4-methyl-2H-1,4-benzoxazin-3(4H)-one
6-{2-[4-(6-Fluoro-2-methyl-5-quinolinyl)-1-piperazinyl]-1-hydroxyethyl}-4-methyl-2H-1,4-benzoxazin-3(4H)-one
4-Methyl-6-{2-[4-(2-methyl-5-quinolinyl)hexahydro-1H-1,4-diazepin-1-yl]ethyl}-2H-1,4-benzoxazin-3(4H)-one
4-Methyl-6-{2-[3-methyl-4-(2-methyl-5-quinolinyl)-1-piperazinyl]ethyl}-2H-1,4-benzoxazin-3(4H)-one
6-{2-[4-(8-Chloro-2-methylquinolin-5-yl)-piperazine-1-yl]-ethyl}-4-methyl-4H-benzo[1,4]oxazine-3-one
6-{2-[4-(8-Fluoro-2-methyl-quinolin-5-yl)-piperazine-1-yl]-ethyl}-4-methyl-4H-benzo[1,4]oxazine-3-one
6-{2-[4-(2-Methyl-1H-indol-4-yl)piperazin-1-yl]ethanoyl}-4H-benzo[1.4]oxazin-3-one
6-{1-Hydroxy-2-[4-(2-methyl-1H-indol-4-yl)piperazinyl]ethyl}-2H-benzo[1.4]oxazin-3-one
6-{1-Fluoro-2-[4-(2-methyl-1H-indol-4-yl)piperazinyl]ethyl}-2H-benzo[1.4]oxazin-3-one
6-{2-[4-(7-Fluoro-2-methyl-5-quinolinyl)-1-piperazinyl]-1-hydroxyethyl}-2-H-1,4-benzoxazin-3-(4H)-one
6-{2-[4-(2-Methyl-5-quinolinyl)-1-piperadinyl}-ethanoyl}-2H-1,4-benzoxazin-3(4H)-one
6-{1-Hydroxy-2-[4-(2-methyl-5-quinolinyl)-1-piperadinyl}-ethyl}-2H-1,4-benzoxazin-3(4H)-one
6-{2-[4-(7-Fluoro-2-methylquinolin-5-yl)piperidin-1-yl]ethyl}-4-H-benzo[1,4]-oxazin-3-one
6-{2-[4-(6-Fluoro-2-methyl-5-quinolinyl)-1-piperazinyl]-1-hydroxyethyl}-2H-1,4-benzoxazin-3(4H)-one
6{2-[4(8-Fluoro-2-methyl-5-quinolinyl)-1-piperazinyl]ethyl}-2H-1,4-benzoxazin-3-(4H)-one
6-{2-[4-2-Quinoxalinyl)-1-piperazinyl]ethyl}-2H-1,4-benzoxazin-3-(4H)-one
4-Methyl-8-{2-[(2R)-2-methyl-4-(2-methyl-5-quinolinyl)-1-piperazinyl]ethyl}-2H-1,4-benzoxazin-3(4H)-one
4-Methyl-8-{2-[(2S)-2-methyl-4-(2-methyl-5-quinolinyl)-1-piperazinyl]ethyl}-2H-1,4-benzoxazin-3(4H)-one
6-{2-[4-(7-Chloro-2,3-dihydro-1,4-benzodioxin-5-yl)-1-piperazinyl]ethyl}-2H-1,4-benzoxazin-3(4H)-one
6-{2-[4-(7-Fluoro-2,3-dihydro-1,4-benzodioxin-5-yl)-1-piperazinyl]ethyl}-2H-1,4-benzoxazin-3(4H)-one
6-{2-[4-(7-Bromo-2,3-dihydro-1,4-benzodioxin-5-yl)-1-piperazinyl]ethyl}-2H-1,4-benzoxazin-3(4H)-one
8-{4-[2-(3-Oxo-3,4-dihydro-2H-1,4-benzoxazin-6-yl)ethyl]-1-piperazinyl}-2,3-dihydro-1,4-benzodioxin-6-carbonitrile
   and pharmaceutically acceptable salts thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Certain compounds of formula (I) are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof. For compounds of formula (I) where R¹ is a C₃₋₆alkenyl group, the compounds may also exist as geometric isomers around the double bond. The present invention includes within its scope all such isomers, including mixtures.

In a further aspect, this invention provides a process for the preparation of a compound of formula (I) or a pharmaceutically acceptable salt thereof, which process comprises:

(a) reacting a compound of formula (II):

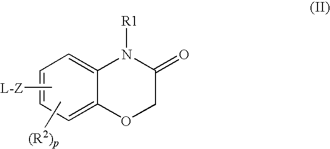

wherein R1, R2, p and Z are as defined in formula (I), and L is a leaving group, with a compound of formula (III):

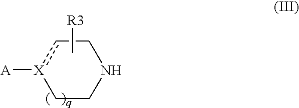

wherein A, R3, [symbol] , X and q are as defined in formula (I); or
(b) the reduction and concominant cyclisation of a compound of formula (IV):

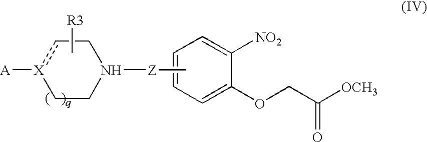

in which A, X, R3, [symbol] , q and Z are as defined in formula (I);
and optionally thereafter for each of process (a) or (b):
removing any protecting groups, and/or
converting a compound of formula (I) into another compound of formula (I), and/or
forming a pharmaceutically acceptable salt.

For process (a), the reaction of a compound of formula (II) and (III) is carried out in the presence of a base such as sodium carbonate or potassium carbonate, in the presence of sodium iodide in a suitable solvent, such as NMP or MIBK at an elevated temperature.

For process (b), the reduction and concominant cyclisation of a compound of formula (IV) is carried out in the presence of a reducing agent such as iron powder in glacial acetic acid.

Compounds of formula (I) can be converted into further compounds of formula (I) using standard techniques. For example, and by way of illustration rather than limitation, for compounds of formula (I) wherein R1 is hydrogen it may be possible to introduce a C₁₋₆alkyl group by conventional alkylation using 1 molar equivalent of a C₁₋₆alkylhalide and 1 molar equivalent of a suitable base in an inert solvent.

Compounds of formulae (II)-(IV) are commercially available, may be prepared according to procedures described herein, by known literature methods, or by analogous procedures thereto.

It will be appreciated by those skilled in the art that it may be necessary to protect certain reactive substituents during some of the above procedures. Standard protection and deprotection techniques, such as those described in Greene T. W. Protective groups in organic synthesis, New York, Wiley (1981), can be used. For example, primary amines can be protected as phthalimide, benzyl, t-butyloxycarbonyl, benzyloxycarbonyl or trityl derivatives. Carboxylic acid groups can be protected as esters. Aldehyde or ketone groups can be protected as acetals, ketals, thioacetals or thioketals. Deprotection of such groups is achieved using conventional procedures well known in the art. For example, protecting groups such as t-butyloxycarbonyl may be removed using an acid such as hydrochloric or trifluororoacetic acid in a suitable solvent such as dichloromethane, diethylether, isopropanol or mixtures thereof.

It will be further appreciated that compounds of formula (II)-(IV) and any precursors thereto may have one or more chiral centres. Enantiomeric or diastereomeric mixtures of such compounds may be separated using conventional methods, for example by chromatography or by resolution by means of diastereomeric salt formation.

Pharmaceutically acceptable salts may be prepared conventionally by reaction with the appropriate acid or acid derivative.

The present invention also provides a pharmaceutical composition, which comprises a compound of formula (I), or formula (Ia) as defined above and a pharmaceutically acceptable carrier or excipient.

In a further aspect, the present invention provides a process for preparing a pharmaceutical composition, the process comprising mixing a compound of formula (I) or formula (Ia) as defined above and a pharmaceutically acceptable carrier or excipient.

The affinities of the compounds of this invention for 5-HT_{1A}, 5-HT_{1B} and 5-HT_{1D} receptors can be determined by the following assay. CHO cells expressing 5-HT_{1A} receptors (4×10⁷ cells/ml) are homogenised in Tris buffer and stored in 1 ml aliquots. CHO cells expressing 5-HT_{1B} receptors (4×10⁷ cells/ml) are homogenised in Tris buffer and stored in 1.5 ml aliquots. CHO cells expressing 5-HT_{1D} receptors (1×10⁸/ml) are homogenised in Tris buffer and stored in 1 ml aliquots. 0.4 ml of a cell suspension is incubated with [³H]-5-HT (4 nM) for 5-HT_{1B/1D} receptors and [³H]WAY100635 (1 nM) for 5-HT_{1A} receptors in Tris Mg HCl buffer (pH 7.7) and test drug, at 37° C. for 45 minutes. Each test drug is tested at 10 concentrations (0.01 mM to 0.3 nM final concentration), with non-specific binding defined using 0.01 mM 5-HT. The total assay volume is 0.5 ml. Incubation is stopped by rapid filtration using a Packard Filtermate and radioactivity measured by Topcount scintillation counting. pKi values are calculated from the IC₅₀ generated by an iterative least squares curve fitting programme.

All the Example compounds shown below were tested according to the radioligand binding assay described above and were found to have pKi values >6.0 at 5-HT_{1A} receptors, with many showing a considerably higher affinity (having pKi values in the range 8.0-10.0) Certain compounds of this invention also demonstrate comparable affinity for 5-HT_{1B} and 5-HT_{1D} receptors.

The intrinsic activity of the compounds of this invention can be determined according to the following assay. HEK293 cell membranes stably expressing human 5-HT_{1A} receptors and CHO cell membranes stably expressing human 5-HT_{1B} receptors are homogenised in HEPES/EDTA buffer and stored in 1 ml aliquots, and [³⁵S]GTPγS binding studies are carried out essentially as described by Lazareno et al., (Life Sci., 1993, 52, 449) with some minor modifications. Membranes from 10⁶ cells are pre-incubated at 30° C. for 30 minutes in 20 mM HEPES buffer (pH 7.4) in the presence of MgCl₂ (3 mM), NaCl (100 mM), GDP (10 μM) and ascorbate (0.2 mM), with or without test compounds. The reaction is started by the addition of 50 μl of [³⁵S]GTPγS (100 μM, assay concentration) followed by a further 30 minutes incubation at 30° C. Non-specific binding is determined using nonradiolabelled GTPγS (20 μM) added prior to the membranes. The reaction is terminated by rapid filtration through Whatman GF/B grade filters followed by 5×1 ml washes with ice cold HEPES (20 mM)/MgCl₂ (3 mM) buffer. Radioactivity is measured using liquid scintillation spectrometry. This procedure is hereafter referred to as the [³⁵S]GTPγS functional assay.

It has been found, using the [³⁵S]GTPγS functional assay, that certain compounds of formula (I) appear to be antagonists at 5-HT₁ type receptors whilst others appear to be inverse agonists, agonists or partial agonists.

The efficacy of the compounds of this invention to inhibit the re-uptake of serotonin can be measured in a 5-HT uptake assay by measurement of uptake of [³H]-5-HT into LLCPK cells expressing human or rat serotonin transporters. In brief, cells are harvested and plated onto 96-well plates (10,000 cells per well). 24 hr later cells are washed 2× with HBSSH (Hanks' balanced salt solution +20 mM HEPES). 50 ul of test compound or vehicle is added to each well and incubated for 10 min. Subsequently, [³H]5-HT (final concentration 25 nM) is added and the test mixture is incubated for a further 7 min. The reaction is terminated by aspiration of test mixture and the cells are washed 6× with HBSSH. 50 ul of scintillation cocktail (Microscint-20, Packard) is added onto the cells and the top and bottom of the plate is sealed. Plates are read, 30 min later, in a Packard TopCount.

Some of the Example compounds tested according to this uptake assay were found to have potency at the uptake site of pIC₅₀ of >6.0. Some showed a considerably higher potency (pIC₅₀>7.0).

Certain compounds of formula (I), formula (Ia) and formula (Ib) as defined above demonstrate both affinity for the 5-HT_{1A} receptor (or affinity for 5-HT_{1A}, 5-HT_{1B} and 5-HT_{1D} receptors) and potency at the 5-HT uptake site in the higher ranges indicated above.

Compounds of the present invention are of use in the treatment of certain CNS disorders, particularly serotonin-related disorders such as depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, vascular dementia with depressed mood, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), schizophrenia, panic disorder, agoraphobia, social phobia, obsessive compulsive disorder, post-traumatic stress disorder, pain (particularly neuropathic pain), memory disorders (including dementia, amnesic disorders and age-associated memory impairment) disorders of eating behaviours (including anorexia nervosa and bulimia nervosa) sexual dysfunction, premature ejaculation, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof, motor disorder such as Parkinson's disease, dementia in Parkinson's disease, neuroleptic-induced Parkinsonism and tardive dyskinesias, as well as other psychiatric disorders.

Compounds of the present invention may also have utility in the treatment of certain gastrointestinal disorders such as irritable bowel syndrome, Crohn's disease, ulcerative colitis, non-steroidal anti-inflammatory drug induced damage.

It is to be understood that the term “treatment” as used herein includes amelioration of established symptoms as well as prevention.

Thus, the present invention provides a compound of formula (I) or formula (Ia) as defined above or a pharmaceutically acceptable salt thereof for use in therapy.

In order to use the compounds of the present invention in therapy, they will normally be formulated into a pharmaceutical composition in accordance with standard pharmaceutical practice. The present invention also provides a pharmaceutical composition, which comprises a compound of formula (I) or formula (Ia) as defined above or a pharmaceutically acceptable salt thereof and a pharmaceutically acceptable carrier or excipient.

In a further aspect, the present invention provides a process for preparing a pharmaceutical composition, the process comprising mixing a compound of formula (I) or formula (Ia) as defined above or a pharmaceutically acceptable salt thereof and a pharmaceutically acceptable carrier or excipient.

As indicated above, DT-2429253-A1 discloses certain benzoxazinone compounds. However, these compounds have not previously been disclosed to have utility in the treatment of serotonin-related disorders.

Accordingly, the present invention provides a compound of formula (Ib) or a pharmaceutically acceptable salt thereof:

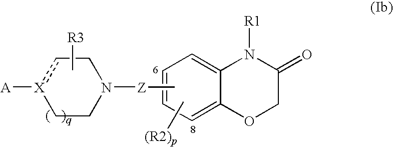

wherein:
A is a bicyclic 6, 5 or 6,6 aromatic or heteroaromatic group which is optionally substituted by 1-4 substituents, which substituents may be the same or different, and which are selected from the group consisting of halogen, hydroxy, cyano, nitro, trifluoromethyl, trifluoromethoxy, C₁₋₆alkyl, trifluoromethanesulfonyloxy, pentafluoroethyl, C₁₋₆alkoxy, arylC₁₋₆alkoxy, C₁₋₆alkylthio, C₁₋₆alkoxyC₁₋₆alkyl, C₃₋₇cycloalkylC₁₋₆alkoxy, C₁₋₆alkanoyl, C₁₋₆alkoxycarbonyl, C₁₋₆alkylsulfonyl, arylsulfonyl, arylsulfonyloxy, C₁₋₆alkylsulfonamido, C₁₋₆alkylamido, arylsulfonamido, arylcarboxamido, aroyl, arylC₁₋₆alkanoyl, and a group Ar¹—B, wherein B represents a single bond, O, S or CH₂ and Ar¹ represents a phenyl or a monocyclic heteroaromatic group, said Ar¹ group being optionally substituted by 1-3 substituents, which may be the same or different, and which are selected from the group consisting of a halogen, hydroxy, cyano, trifluoromethyl, C₁₋₆alkyl, C₁₋₆alkoxy or C₁₋₆alkanoyl;
R1 is hydrogen, C₁₋₆alkyl, haloC₁₋₆alkyl, C₃₋₇cycloalkyl, C₃₋₇cycloalkylC₁₋₆alkyl, C₃₋₆alkenyl, C₃₋₆alkynyl or arylC₁₋₆alkyl;
R2 is independently halogen, C₁₋₆alkyl, cyano, haloC₁₋₆alkyl, C₁₋₆alkanoyl, C₁₋₆alkoxy or hydroxy;
p is 0, 1 or 2;
R3 (a) is a group —(R4)r wherein R4 is selected from the group consisting of: C₁₋₆alkyl, halogen, hydroxy, oxo, cyano, nitro, C₁₋₄alkoxy, haloC₁₋₄alkyl, haloC₁₋₄alkoxy, arylC₁₋₄alkoxy, C₁₋₄alkylthio, hydroxyC₁₋₄alkyl, C₁₋₄alkoxyC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, aroyl, aroylC₁₋₄alkyl, arylC₁₋₄alkanoyl, C₁₋₄acyl, aryl, arylC₁₋₄alkyl, C₁₋₄alkylaminoC₁₋₄alkyl and a group R30R31N- (where each of R30 and R31 independently represents a hydrogen atom or a C₁₋₄alkyl group or where appropriate R30R31 forms part of a C₃₋₆azacyloalkane or C₃₋₆(2-oxo)azacycloalkane ring), and r is 0, 1, 2 or 3; or
(b) forms a bridge across the ring, the bridge consisting of a chain of 1 to 3 atoms, the bridge being optionally substituted by one, two or three groups selected from halogen, oxo, C₁₋₆alkyl, cyano, haloC₁₋₆alkyl, C₁₋₆alkanoyl, C₁₋₆alkoxy or hydroxy; or
(c) is a chain of 1 to 3 atoms optionally substituted by halogen, C₁₋₆alkyl, cyano, haloC₁₋₆alkyl, C₁₋₆alkanoyl, C₁₋₆alkoxy or hydroxy, the other end of the chain being attached to an available carbon atom in Z;

### X is CH, N or C;

==== represents a single bond when X is CH or N; and ==== represents a double bond when X is C;
   q is 0, 1 or 2, wherein when q is 0, X is not N; and
   Z is attached to the 6-position or the 8-position of the benzoxazinone group and is a 3 to 7 membered cycloalkylene group, 3 to 7 membered cycloalkenylene group, —(CH═CH)— or a group

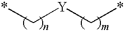

wherein m and n are independently 0, 1 or 2, and Y is a single bond, 3 to 7 membered cycloalkylene group, 3 to 7 membered cycloalkenylene group, —(CH═CH)—, —C(═O)—, —C(═CH₂)—, oxygen, or a methylene group optionally substituted by one or two groups selected from halogen, C₁₋₆alkyl, cyano, haloC₁₋₆alkyl, C₁₋₆alkanoyl, C₁₋₆alkoxy or hydroxy;
for use in the treatment of a serotonin-related disorder.

The serotonin-related disorder may be depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, vascular dementia with depressed mood, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), schizophrenia, panic disorder, agoraphobia, social phobia, obsessive compulsive disorder and post-traumatic stress disorder, pain (particularly neuropathic pain), memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof, motor disorders such as Parkinson's disease, dementia in Parkinson's disease, neuroleptic-induced Parkinsonism and tardive dyskinesias, as well as other psychiatric disorders.

Preferably the disorder is depression or anxiety.

All preferred features of formula (I) and (Ia) apply to compounds of formula (Ib) mutatis mutandis.

The present invention also provides use of a compound of formula (Ib) or a pharmaceutically acceptable salt thereof in the preparation of a medicament for the treatment of a serotonin-related disorder for example as defined above. Preferably the disorder is depression or anxiety.

Furthermore, the present invention provides a method of treatment of a serotonin-related disorder for example as defined above, comprising administering to a mammal in need thereof a safe and effective amount of a compound of formula (Ib) or a pharmaceutically acceptable salt thereof. Preferably the disorder is depression or anxiety.

Compounds of the present invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants lubricants (e.g. magnesium stearate, talc or silica); disintegrants (e.g. potato starch or sodium starch glycollate); and acceptable wetting agents (e.g. sodium lauryl sulfate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of the compounds of the invention.

### Description 1

### 2-Methylquinolin-5-yl trifluoromethanesulfonate (D1)

A solution of 2-methylquinolin-5-ol (2.5 g; 15.7 mmol) (WO/0234754) in dry DCM (25 mL) and pyridine (6.4 mL; 5 eq.) was cooled to 0° C. and trifluoromethanesulfonic anhydride (4.2 mL; 1.6 eq) was added dropwise over 10 minutes. The reaction mixture was stirred under nitrogen at r.t. for 1 h, then poured into water (20 mL) and extracted into ethyl acetate (3×15 mL). The organic layers were combined, dried (Na₂SO₄) and concentrated in vacuo. The crude product was purified by flash chromatography, eluting with 40% ethyl acetate in cyclohexane to afford the title compound (D1) as a yellow solid (4.2 g; yield 92%).

MS; (ES) m/z: 292.3 [MH⁺]. C₁₁H₈F₃NO₃S requires 291.

¹H-NMR (300 MHz, DMSO) δ: 8.05 (d, 1H), 7.85 (d, 1H), 7.64 (t, 1H), 7.48 (d, 1H), 7.43 (d, 1H), 2.48 (s, 3H).

### Description 2

### tert-Butyl 4-(2-methylquinolin-5-yl)piperazine-1-carboxylate (D2)

tert-Butyl 1-piperazinecarboxylate (3.8 g; 1.2 eq.), caesium carbonate (8.4 g; 1.5 eq.), palladium acetate (0.31 g; 0.08 eq.) and 2,2′-bis(diphenylphosphino)-1,1′-binaphthyl (1.3 g; 0.12 eq.) were added to a solution of 2-methyl-quinolin-5-yl-trifluoro-methanesulfonate (D1) (5.0 g, 0.017 mol) in dry toluene (150 mL) under nitrogen. The reaction mixture was stirred at reflux under nitrogen for 8 h. The reaction was then cooled to r.t. and quenched with a saturated aqueous solution of ammonium chloride (100 mL) and then extracted with ethyl acetate (3×100 mL). The organic layers were combined, dried (Na₂SO₄) and concentrated in vacuo. The crude product was purified by flash chromatography, eluting with 30% ethyl acetate in cyclohexane to afford the title compound (D2) as a yellow oil (4.74 g; yield 84%).

MS; (ES) m/z: 328.4 [MH]⁺. C₁₉H₂₅N₃O₂ requires 327.

¹H-NMR (500 MHz, CDCl₃) δ: 8.40 (d, 1H), 7.76 (d, 1H), 7.61 (t, 1H), 7.29 (d, 1H), 7.06 (d, 1H), 3.69 (bs, 4H), 3.03 (bs, 4H), 2.74 (s, 3H), 1.51 (s, 9H).

### Description 3

### 2-Methyl-5-piperazin-1-ylquinoline (D3)

tert-Butyl 4-(2-methyl-quinolin-5-yl)piperazine-1-carboxylate (D2) (1.1 g, 3.9 mmol) in a 25% solution of trifluoroacetic acid in DCM (10 mL) was stirred at r.t. under nitrogen for 3 h. The reaction mixture was concentrated in vacuo and filtered through a 20 g SCX cartridge to afford the title compound (D3) as a yellow solid (0.74 g; yield 96%).

MS; (ES) m/z: 228.4 [MH]⁺. C₁₄H₁₇N₃ requires 227.

¹H-NMR (300 MHz, DMSO) δ: 8.34 (d, 1H), 7.57 (m, 2H), 7.35 (m, 1H), 7.06 (m, 1H), 2.93 (bm, 8H), 2.62 (s, 3H).

### Description 4

### 6-(2-Chloroethyl)-4H-benzo[1,4]oxazin-3-one (D4)

A solution of 6-(2-chloroethanoyl)-2H-benz[1,4]oxazin-3-one (5.0 g, 22.16 mmol) in trifluoroacetic acid (30 mL) was cooled to 0° C. and triethylsilane (7.8 mL; 2.3 eq) was added dropwise over 2 minutes. The reaction mixture was stirred under nitrogen at 0° C. for 10 minutes, warmed to 45° C. for 20 minutes and then allowed to stir at r.t. overnight. It was then poured into ice/saturated aqueous sodium bicarbonate (20 mL) and extracted into ethyl acetate (3×15 mL). The organic layers were combined, dried (Na₂SO₄) and concentrated in vacuo. The crude product was washed with hexane (30 mL), stirred vigorously for 3 h, filtered and dried (Na₂SO₄) to give the title compound (D4) as a white solid (4.3 g; yield 91%).

MS; (ES) m/z: 212.1 [MH]⁺. C₁₀H₁₀ClNO₂ requires 211.

¹H-NMR (500 MHz, CDCl₃) δ: 8.10 (bs, 1H), 6.88 (d, 1H), 6.82 (dd, 1H), 6.65 (d, 1H), 4.57 (s, 2H), 3.65 (t, 2H), 2.98 (t, 2H).

### Description 5

### 2,7-Dimethyl-5,6,7,8-tetrahydroquinoline-5-one (D5)

A solution of 3-amino-5-methylcyclohex-2-enone (5 g; 40 mmol) in 4-methoxy-3-buten-2-one (7 mL) was heated under nitrogen from 100 to 160° C. over 0.5 h, then at 170° C. for 2 h using a Dean Stark set up. The crude reaction mixture was purified by SPE-SI bond elute, eluting with 20% ethyl acetate in cyclohexane to afford the title compound (D5) as a yellow-orange oil (5.7 g; yield 81%).

MS; (ES) m/z: 176.1 [MH⁺]. C₁₁H₁₃NO requires 175.

¹H-NMR (300 MHz, CDCl₃) δ: 8.32 (d, 1H), 7.18 (1H, d), 3.31 (d, 2H), 2.85 (d, 2H), 2.67 (s, 3H), 2.43 (m, 1H), 1.22 (s, 3H).

### Description 6

### 6-Bromo-2,7-dimethyl-5,6,7,8-tetrahydroquinoline-5-one (D6)

A solution of 2,7-dimethyl-5,6,7,8-tetrahydroquinoline-5-one (D5) (2 g; 11.4 mmol) in hydrobromic acid (12 mL of 48% in water) was treated dropwise with bromine (0.6 mL, 1 eq) at 60° C. under vigorous stirring. The resulting mixture was stirred at 60° C. for 1 h then evaporated in vacuo to give a waxy solid, which was triturated with a 1:1 solution of ether-isopropanol to afford the title product (D6) as a white powder (3 g; yield 100%).

MS; (ES) m/z: 254/256 [MH⁺]. C₁₁H₁₂NOBr requires 254.

¹H-NMR (300 MHz, CDCl₃) δ: 8.79 (d, 1H), 7.64 (d, 1H), 4.59 (d, 1H), 3.11 (s, 3H+2H), 2.37 (m, 1H), 1.31 (d, 3H).

### Description 7

### 5-Hydroxy-2,7-dimethylquinoline (D7)

A mixture of 6-bromo-2,7-dimethyl-5,6,7,8-tetrahydroquinoline-5-one (D6) (3 g; 11.7 mmol), lithium carbonate (1.73 g, 2 eq.), lithium bromide (1.01 g, 1 eq.) and DMF (30 mL) was heated at 150° C. under nitrogen for 2 h. The mixture was cooled and then evaporated in vacuo. The crude reaction mixture was twice purified through 50 g SCX columns to afford the title product (D7) as a yellow solid (1.3 g; 63%).

MS; (ES) m/z: 174.0 [MH⁺]. C₁₁H₁₁NO requires 173.

¹HNMR (600 MHz, DMSO) δ: 10.18 (s, 1H), 8.23 (d, 1H), 7.18 (d, 1H), 7.10 (s, 1H), 6.63 (s, 1H), 2.54 (s, 3H), 2.34 (s, 3H).

### Description 8

### (2,7-Dimethylquinolin-5-yl)piperazine (D8)

The title compound (D8) was prepared from 5-hydroxy-2,7-dimethylquinoline (D7) by the general methods described above for the preparation of D3.

¹H-NMR (300 MHz, CDCl₃) δ: 8.35 (d, 1H), 7.55 (s, 1H), 7.20 (d, 1H), 6.90 (s, 1H), 3.15 (br m, 4H), 3.05 (br m, 4H), 2.75 (s, 3H), 2.50 (s, 3H), 2.30 (br s, 1H).

### Description 9

### 7-Chloro-2-methyl-5-piperazin-1-ylquinoline (D9)

The title compound (D9) was prepared from 7-chloro-5-hydroxy-2-methylquinoline (WO/0234754) according to the general method described for the preparation of D3.

MS; (ES) m/z: 262.1 [MH]⁺. C₁₄H₁₆ClN₃ requires 261.

¹H-NMR (300 MHz, DMSO) δ: 8.36 (d, 1H), 7.61 (d, 1H), 7.40 (d, 1H), 6.92 (d, 1H, 3.32 (m, 4H), 2.93 (m, 4H), 2.62 (s, 3H).

### Description 10

### 5-Fluoro-2-methyl-3,4-dihydroquinazoline (D10)

A stirred solution of 2-amino-6-fluorobenzylamine (1.1 g, 7.9 mmol) and triethyl orthoacetate (1.4 g, 8.6 mmol, 1.1 eq) in ethanol (30 mL) was heated at 80° C. overnight. The reaction mixture was allowed to cool to r.t. and the solvent concentrated in vacuo. The crude reaction mixture was triturated with ether and filtered to afford the title compound (D10) as a white solid (0.74 g, yield 57%).

MS; (ES) m/z: 165.1 [MH⁺]. C₉H₉FN₂ requires 164.

¹H-NMR (300 MHz, CDCl₃) δ: 7.1 (q, 1H), 6.7 (t, 2H), 4.7 (s, 2H), 2.0 (s, 3H).

### Description 11

### 5-Fluoro-2-methylquinazoline (D11)

To a stirred solution of 5-fluoro-2-methyl-3,4-dihydroquinazoline (D10) (0.74 g, 4.5 mmol) in chloroform (100 mL) manganese dioxide (2.0 g, 23 mmol, 5 eq.) was added portionwise. The reaction mixture was stirred at r.t. overnight, then a further portion of manganese dioxide (2.0 g, 23 mmol, 5 eq.) was added and stirring continued for 6 h. The reaction mixture was then filtered through a celite pad, which was then washed with DCM (50 mL). The combined organics were concentrated in vacuo to afford the title compound (D11) as a yellow solid (0.72 g, yield 98%).

MS; (ES) m/z: 163.1 [MH⁺]. C₉H₇FN₂ requires 162.

¹H-NMR (300 MHz, CDCl₃) δ: 9.6 (s, 1H), 7.8 (m, 2H), 7.2 (t, 1H), 2.9 (s, 3H).

### Description 12

### 2-Methyl-5-piperazin-1-ylquinazoline (D12)

To a solution of 5-fluoro-2-methylquinazoline (D11) (2 g; 12.3 mmol) in dry DMF (10 mL) triethylamine (3.4 mL; 2 eq.) and piperazine (11 g; 10 eq.) were added. The reaction mixture was stirred under nitrogen at 120° C. for 4 h., then poured into water (10 mL) and extracted with ethyl acetate (5×15 mL). The organic layers were combined, dried (Na₂SO₄) and concentrated in vacuo. The crude product was purified by SCX cartridge to afford the title compound (D12) as a yellow solid. (1.8 g; yield 64%).

MS; (ES) m/z: 229.2 [MH⁺]. C₁₁H₈F₃NO₃S requires 228.

¹H-NMR (300 MHz, CDCl₃) δ: 9.58 (s, 1H), 7.89 (t, 1H), 7.62 (d, 1H), 7.27 (d, 1H), 3.25 (m, 8H); 2.91 (s, 3H).

### Description 13

### 2-Methyl-5-(3-methylpiperazin-1-yl)quinoline (D13)

2-Methylpiperazine (40.8 mg; 0.40 mmol; 1.2 eq.), cesium carbonate (164 mg; 0.5 mmol; 1.5 eq.), palladium acetate (6 mg; 0.028 mmol; 0.08 eq.) and 2,2′-bis(diphenylphosphino)-1,1′-binaphthyl (26 mg; 0.042 mmol; 0.12 eq.) were added to a solution of 2-methylquinolin-5-yltrifluoromethanesulfonate (D1) (100 mg, 0.34 mmol; 1 eq) in dry toluene (1.50 mL) under nitrogen. The reaction mixture was stirred at reflux under nitrogen for 8 h. A further addition of 2-methylpiperazine (40.8 mg; 0.40 mmol; 1.2 eq) and palladium acetate (6 mg; 0.028 mmol; 0.08 eq.) was then made to the reaction mixture followed by heating at reflux for a further 2 h. The reaction was cooled to r.t. and quenched with a saturated aqueous solution of ammonium chloride (100 mL) and then extracted into ethyl acetate (3×50 mL). The organic layers were combined, dried (Na₂SO₄) and concentrated in vacuo. The crude product was purified by SPE cartridge (Si, 2 g), eluting with 5% methanol in dichloromethane to afford the title compound as a red oil (56 mg; yield 69%).

MS; (ES) m/z: 241.34 [MH]⁺. C₁₅H₁₉N₃ requires 242.4.

¹H-NMR (500 MHz, CDCl₃) δ: 8.36 (d, 1H), 7.77 (d, 1H), 7.61 (t, 1H), 7.29 (d, 1H), 7.12 (d, 1H), 3.45 (t, 1H), 3.3-2.8 (d/t, 1/1H), 3.3-3.1 (m/m, 1/1H), 3.3-2.75 (m/m, 1/1H), 2.74 (s, 3H), 1.36 (d, 3H).

### Description 14

### 2-Methyl-5-(2-methylpiperazin-1-yl)quinoline (D14)

3-Methylpiperazine-1-carboxylic acid tert-butyl ester (160 mg; 0.80 mmol; 2 eq.) (prepared as reported in J. Med. Chem. 1993, 36, 690-698), cesium carbonate (195 mg; 0.6 mmol; 1.5 eq.), palladium acetate (9 mg; 0.04 mmol; 0.10 eq.) and 2,2′-bis(diphenylphosphino)-1,1′-binaphthyl (38 mg; 0.06 mmol; 0.15 eq.) were added to a solution of 2-methylquinolin-5-yl-trifluoromethanesulfonate (D1) (117 mg, 0.4 mmol; 1 eq) in dry toluene (2.5 mL) under nitrogen. The reaction mixture was stirred at reflux under nitrogen for 10 h. The reaction was cooled and filtered through a pad of celite which was then washed with DCM (50 mL). The filtrates was concentrated in vacuo and the crude product was purified by SPE cartridge (Si, 2 g), eluting with 5% ethylacetate in cyclohexane to afford 3-methyl-4-(2-methylquinolin-5-yl)piperazin-1-carboxylic acid tert-butyl ester as a yellow oil (84 mg; yield 62%). MS; (ES) m/z: 341.45 [MH]⁺. C₂₀H₂₇N₃O₂ requires 342.4. ¹H-NMR (300 MHz, CDCl₃) δ: 8.5 (d, 1H), 7.77 (d, 1H), 7.61 (t, 1H), 7.29 (d, 1H), 7.12 (d, 1H), 3.8-3.6, m/m, 2H), 3.4-3.3 (m, 1H), 3.2-3.1 (m, 1H), 3.1-2.9 (m, 2H), 2.74 (s, 3H), 1.45 (s, 9H), 1.36 (d, 3H). This compound (84 mg) was dissolved in a mixture 3:1 of trifluoroacetic acid: DCM (4 mL) and stirred at r.t. for 6 h. The solvent was evaporated in vacuo and the residue purified on SCX cartridge (1 g) to afford the title compound (D14) (44 mg; yield 76%)

MS; (ES) m/z: 241.45 [MH]⁺. C₁₅H₁₉N₃ requires 242.4.

¹H-NMR (300 MHz, CDCl₃) δ: 8.5 (d, 1H), 7.77 (d, 1H), 7.61 (t, 1H), 7.29 (d, 1H), 7.12 (d, 1H), 3.3 (m, 4H), 3.15 (m, 4H), 2.74 (s, 3H), 1.9 (m, 2H).

### Description 15

### 2-Methyl-5-(3-methylpiperazin-1-yl)quinazoline (D15)

A solution of 2-methyl-5-fluoroquinazoline (100 mg; 0.616 mmol; 1 eq), 2-methylpiperazine (310 mg; 3.083 mmol; 5 eq) and triethylamine (0.17 mL; 1.23 mmol; 2 eq) in dry DMF (2.5 mL) was heated at 120° C. for 5 h. The yellow solution was cooled and the solvent was evaporated in vacuo. The crude material was purified on SPE cartridge (Si; 2 g) eluting with a gradient from 100% dichloromethane to 85% dichloromethane: 1% NH₄OH 2M sol in methanol to afford the title compound (D15) (85 mg; yield 57%).

MS; (ES) m/z: 243.3 [MH⁺]. C₁₄H₁₈N₄ requires 242.32.

¹H NMR (300 MHz, CDCl₃) δ: 9.48 (s, 1H), 7.81 (t, 1H), 7.48 (d, 1H), 7.10 (d, 1H), 3.03 (m, 1H), 3.23-2.98-2.76-2.41 (m, 6H), 2.72 (s, 3H) 1.01 (d, 3H).

### Description 16

### 4-(2-Methylquinolin-5-yl)-3,6-dihydro-2H-pyridine-1-carboxylic acid tert-butyl ester (D16)

A mixture of 1(2H)-pyridinecarboxylic acid, 3,6-dihydro-4-(4,4,5,5-tetramethyl-1,3,2 dioxaborolan-2-yl)-1,1-dimethylethyl ester (Tetrahedron Letters 2000, 41, 3705-3708) (0.56 g, 1.8 mmol), 2-methylquinolin-5-yl trifluoromethanesulfonate (D1) (0.5 g, 1.72 mmol), [1,1′-bis(diphenylphosphino)ferrocene]palladium(II) chloride (140 mg, 0.17 mmol) and potassium carbonate (0.713 g, 5.1 mmol) in dry DMF (20 mL) was heated at 80° C. under nitrogen for 3 h. The DMF was removed in vacuo and the residue partitioned between water (25 mL) and DCM (3×50 mL). The organic extracts were dried (Na₂SO₄) and chromatographed on silica (eluent 30% EtOAc/cyclohexane) to afford the title compound (D16) as a colourless oil (0.35 g, 63%).

MS; (ES) m/z: 325 [MH⁺]. C₂₀H₂₄N₂O₂ requires 324.

¹H-NMR (300 MHz, CDCl₃) δ: 8.15 (d, 1H), 7.90 (d, 1H), 7.57 (t, 1H), 7.22 (m, 2H), 5.70 (br s, 1H), 4.20 (br m, 2H), 3.65 (m, 2H), 2.70 (s, 3H), 2.45 (br m, 2H), 1.50 (s, 9H).

### Description 17

### 2-Methyl-5-(1,2,3,6-tetrahydropyridin-4-yl]quinoline (D17)

A solution of 4-(2-methylquinolin-5-yl)-3,6-dihydro-2H-pyridine-1-carboxylic acid tert-butyl ester (D16) (150 mg, 0.46 mmol) in DCM (10 mL) was treated with trifluoroacetic acid (0.35 mL, 0.46 mmol), stirred for 30 minutes and then stirred at r.t. for 16 h. The reaction mixture was made basic with saturated aqueous NaHCO₃ (10 mL), then the organics were separated, dried (Na₂SO₄) and evaporated to give the title compound (D17) as a white solid (85 mg, 83%).

MS; (ES) m/z: 225 [MH⁺]. C₁₅H₁₆N₂ requires 224.

¹H-NMR (300 MHz, CDCl₃) δ: 8.25 (d, 1H), 7.87 (d, 1H), 7.58 (t, 1H), 7.23 (m, 2H), 5.75 (br m, 1H), 3.57 (m, 2H), 3.25 (m, 2H), 2.70 (s, 3H), 2.40 (m, 2H). NH not observed.

### Description 18

### 4-(2-Methylquinolin-5-yl)piperidine-1-carboxylic acid tert-butyl ester (D18)

A solution of 4-(2-methylquinolin-5-yl)-3,6-dihydro-2H-pyridine-1-carboxylic acid tert-butyl ester (D16) (200 mg, 0.62 mmol) in ethanol (10 mL) was hydrogenated over 10% Pd—C (20 mg) at atmospheric pressure for 20 h at r.t. The reaction mixture was filtered through a celite pad which was then washed with ethanol (2×50 mL). The combined filtrates were then evaporated to give the title compound (D18) as a clear colourless oil (150 mg, 74%).

MS; (ES) m/z: 327 [MH⁺]. C₂₀H₂₆N₂O₂ requires 326.

¹H-NMR (300 MHz, CDCl₃) δ: 8.20 (d, 1H), 7.80 (d, 1H), 7.55 (t, 1H), 7.20 (m, 2H), 4.20 (br m, 2H), 3.25 (br m, 1H), 2.85 (br m, 1H), 2.65 (s, 3H), 1.85 (br m, 2H), 1.65 (br m, 2H), 1.40 (s, 9H), 1.38 (m, 1H).

### Description 19

### 2-Methyl-5-piperidin-4-ylquinoline (D19)

The title compound (D19) was prepared in a similar fashion to Description 17 starting from 4-(2-methylquinolin-5-yl)piperidine-1-carboxylic acid tert-butyl ester (D18) (138 mg, 0.42 mmol) as a pale yellow oil (69 mg, 72%).

¹H-NMR (300 MHz, CDCl₃) δ: 8.25 (d, 1H), 7.85 (d, 1H), 7.60 (t, 1H), 7.35 (d, 1H), 7.25 (d, 1H), 3.10-3.40 (m, 3H), 2.85 (t, 2H), 2.70 (s, 3H), 1.85 (br d, 2H), 1.60-1.80 (m, 2H). MH not observed.

### Description 20

### 4-(2-Methylquinolin-5-yl)-[1,4]diazepane-1-carboxylic acid tert-butyl ester (D20)

Homopiperazine (160 mg; 0.80 mmol; 2 eq.) (prepared as reported in J. Med. Chem. 1993, 36, 690-698), cesium carbonate (195 mg; 0.6 mmol; 1.5 eq.), palladium acetate (9 mg; 0.04 mmol; 0.10 eq.) and 2,2′-bis(diphenylphosphino)-1,1′-binaphthyl (38 mg; 0.06 mmol; 0.15 eq.) were added to a solution of 2-methylquinolin-5-yl-trifluoromethanesulfonate (D1) (117 mg, 0.4 mmol; 1 eq) in dry toluene (2.5 mL) under nitrogen. The reaction mixture was stirred at reflux under nitrogen for 10 h then cooled and filtered through a celite pad which was then washed with DCM (2×50 mL). The combined filtrates were concentrated in vacuo and the crude product was purified by SPE cartridge (Si, 2 g), eluting with 5% ethylacetate in cyclohexane affording the title compound (D20) as a yellow oil (84 mg; yield 62%).

MS; (ES) m/z: 341.45 [MH]⁺. C₂₀H₂₇N₃O₂ requires 342.4.

¹H-NMR (300 MHz, CDCl₃) δ: 8.5 (d, 1H), 7.77 (d, 1H), 7.61 (t, 1H), 7.29 (d, 1H), 7.12 (d, 1H), 3.8-3.6, m/m, 2H), 3.4-3.3 (m, 1H), 3.2-3.1 (m, 1H), 3.1-2.9 (m, 2H), 2.74 (s, 3H), 1.45 (s, 9H), 1.36 (d, 3H).

### Description 21 (D21)

### 5-[1,4]Diazepan-1-yl-2-methylquinoline (D21)

4-(2-Methylquinolin-5-yl)-[1,4]diazepane-1-carboxylic acid tert-butyl ester (D20) (51 mg) was dissolved in a mixture 3:1 of trifluoroacetic acid/DCM (4 mL) and stirred at r.t. for 6 h. The solvent was evaporated in vacuo and the residue purified on SCX cartridge (1 g) to afford the title compound (D21) (35 mg; yield 85%).

MS; (ES) m/z: 241.45 [MH]⁺. C₁₅H₁₉N₃ requires 242.4.

¹H-NMR (300 MHz, CDCl₃) δ: 8.5 (d, 1H), 7.77 (d, 1H), 7.61 (t, 1H), 7.29 (d, 1H), 7.12 (d, 1H), 3.3 (m, 4H), 3.15 (m, 4H), 2.74 (s, 3H), 1.9 (m, 2H).

### Description 22

### 5-[1,4]Diazepan-1-yl-2-methylquinazoline (D22)

A solution of 5-fluoro-2-methylquinazoline (D11) (100 mg; 0.616 mmol; 1 eq), homopiperazine (309 mg; 3.083 mmol; 5 eq) and triethylamine (0.17 mL; 1.23 mmol; 2 eq.) in dry DMF (2.5 mL) was heated at 120° C. for 5 h. The yellow solution was cooled and the solvent evaporated in vacuo. The residue was dissolved in ethylacetate (20 mL) and washed with brine (3×15 mL). The organic layers were combined, dried (Na₂SO₄) and concentrated in vacuo. The crude material was purified on SPE cartridge (Si; 2 g) eluting with a gradient from 100% DCM to 85% DCM 1% NH₄OH 2M sol in methanol to afford the title compound (D22) (50 mg; yield 35%).

MS; (ES) m/z: 243.3 [MH⁺]. C₁₄H₁₈N₄ requires 242.32.

¹H NMR (300 MHz, CDCl₃) δ: 9.5 (s, 1H), 7.7 (t, 1H), 7.45 (d, 1H), 7.05 (d, 1H), 3.45 (t, 4H), 3.1 (t, 4H), 2.85 (s, 3H) 2.05 (5, 2H).

### Description 23

### 6-(2-Chloroethanoyl)-7-fluoro-4H-benzo[1,4]oxazin-3-one (D23)

Aluminum chloride (1.9 g; 2.2 eq) and chloroacetyl chloride (0.6 mL; 1.2 eq) were added at r.t. to a suspension of 7-fluoro-4H-benzo[1,4]oxazin-3-one (1.1 g; 6.58 mmol) in dry 1,2-dichloroethane (10 mL). The reaction mixture was stirred at 80° C. under nitrogen for 3 h, then poured into a saturated aq. solution of ammonium chloride (10 mL) and extracted into ethyl acetate (3×10 mL). The organic layers were combined, dried (Na₂SO₄) and concentrated in vacuo. The crude product was purified by flash chromatography on silica gel, eluting with 60% ethyl acetate in cyclohexane to afford the title compound (D23) as a white solid (0.8 g; yield 50%).

MS; (ES) m/z: 244.1 [MH⁺]. C₁₀H₇ClFNO₃ requires 243.

¹H-NMR (300 MHz, DMSO) δ: 10.58 (s, 1H), 7.41 (d, 1H), 7.05 (d, 1H), 7.48 (d, 1H), 4.95 (d, 2H), 4.74 (s, 2H).

### Description 24

### 6-(2-Chloroethyl)-7-fluoro-4H-benzo[1,4]oxazin-3-one (D24)

The title compound (D24) was prepared in 72% yield according to the experimental procedure described for D4 starting from 6-(2-chloroethanoyl)-7-fluoro-4H-benzo[1,4]oxazin-3-one (D23).

MS; (ES) m/z: 230.2 [MH]⁺. C₁₀H₉ClFNO₂ requires 229.

¹H-NMR (300 MHz, DMSO) δ: 10.72 (s, 1H), 6.87 (d, 1H), 6.81 (d, 1H), 4.56 (s, 2H), 3.77 (t, 2H), 2.96 (t, 2H).

### Description 25

### (4-Bromo-2-nitro-phenoxy)acetic acid methyl ester (D25)

A mixture of 4-bromo-2-nitro-phenol (6.0 g, 27.5 mmol), methyl bromoacetate (5.0 g, 33 mmol), anhydrous potassium carbonate (4.6 g, 33.3 mmol) in DMF (70 mL) was stirred at 50° C. for 4 h. The solvent was removed under vacuum and then the residue was co-evaporated with toluene (3×20 mL), dissolved in DCM (150 mL), washed with water (2×50 mL), 1N sodium hydroxide (1×50 mL), water (2×50 mL) and dried (MgSO₄). The solvent was evaporated to give the title compound (D25) as a yellow solid (7.9 g, 99% yield); ^{δH (}400 MHz, CDCl₃), 3.81 (3H, s) 4.78 (2H, s), 6.89 (1H, d, J=8.8 Hz), 7.63 (1H, dd, J=8.8 Hz, 2.5 Hz), 8.00 (1H, J=2.5 Hz).

### Description 26

### (4-Allyl-2-nitrophenoxy)acetic acid methyl ester (D26)

(4-Bromo-2-nitro-phenoxy)acetic acid methyl ester (D25) (6.8 g, 23.5 mmol), allyl tributyl tin (12 g, 36.2 mmol), tetrakis(triphenylphosphine)palladium(0) (1.39 g, 1.2 mmol) in toluene (100 mL), was stirred at 110° C. for 8 h under argon. The solvent was removed and the residue was dissolved in acetonitrile (300 mL) and washed with petroleum ether (40-60° C.) (4×100 mL). The acetonitrile was evaporated and the crude product was purified by column chromatography on silica gel (eluting with ethyl acetate-hexane gradient) to give the title compound (D26) as a tan oil (4.0 g, 68% yield); MS: m/z (MH⁺)=252/253.

### Description 27

### [4-(3-Hydroxypropyl)-2-nitrophenoxy]acetic acid methyl ester (D27)

To a solution of (4-allyl-2-nitrophenoxy)acetic acid methyl ester (D26) (2.0 g, 8.0 mmol), in THF (30 mL) at 0° C., was added borane-tetrahydrofurane complex (10 mL, 1M solution in THF) dropwise over a period of 2 h. The mixture was stirred at 5-8° C. for 1.5 h, treated with water (15 mL, added slowly), then with sodium perborate tetrahydrate (2.0 g, 13 mmol). The resulting mixture was stirred vigorously at r.t. for 2.5 h, concentrated to a small volume and the residual water solution was extracted with ethyl acetate (4×20 mL). The combined extracts were dried (MgSO₄), the solvent was evaporated and the product was purified by column chromatography on silica gel (eluting with ethyl acetate-dichloromethane gradient) to give the title compound (D27) as a slightly tan oil (0.5 g, 100% pure and 0.166 g mixture of the title compound and the corresponding secondary alcohol, ratio 8:2; 30% yield); MS: m/z (MH⁺)=270/271.

### Description 28

### [4-(3-Methanesulfonyloxypropyl)-2-nitrophenoxy]acetic acid methyl ester (D28)

To a stirred solution of 4-(3-hydroxypropyl)-2-nitrophenoxy]acetic acid methyl ester (D27) (0.5 g, 1.9 mmol) and triethylamine (0.46 g, 4.5 mmol) in DCM (20 mL) at 0° C. was added a solution of methanesulfonyl chloride (0.33 g, 2.9 mmol) in dichloromethane (5 mL), this was then stirred at 5° C. for 3 h. The mixture was diluted with DCM (80 mL), washed with saturated aqueous sodium hydrogen carbonate (2×30 mL), water (1×30 mL) and dried (MgSO₄). The solvent was evaporated to give the title compound (D28) as a cream solid (0.56 g, 87% yield); MS: m/z (MH⁺)=348.

### Description 29

### (4-{3-[4-(2-Methylquinolin-5-yl)piperazin-1-yl]propyl}-2-nitrophenoxy)-acetic acid methyl ester (D29)

A mixture of [4-(3-methanesulfonyloxypropyl)-2-nitrophenoxy]acetic acid methyl ester (D28) (0.21 g, 0.6 mmol), 2-methyl-5-piperazin-1-ylquinoline (D3) (0.23 g, 1 mmol), anhydrous potassium carbonate (0.21 g, 1.5 mmol), sodium iodide (0.09 g, 0.6 mmol), and molecular sieves (0.4 g, 4 A) in DMF (10 mL) was stirred at 80° C. for 1.5 h. The solvent was evaporated, the residue was dissolved in DCM (100 mL) and washed with water (2×20 mL). The solvent was evaporated and the product was purified by column chromatography on silica gel (eluting with methanol-dichloromethane gradient) to give the title compound (D29) as a tan oil, (0.2 g, 69% yield); MS: m/z (MH⁺)=479/481.

### Description 30

### 7-Fluoro-2-methyl-5-piperazin-1-ylquinoline (D30)

A solution of 5,7-difluoro-2-methylquinoline (WO/0234754) (1.2 g, 6.70 mmol) and piperazine (2.5 eq) in dry DMSO (10 mL) was stirred at 95° C. under nitrogen for 24 h. The reaction mixture was worked-up using an SCX cartridge and the residue then purified by flash chromatography on silica gel, eluting with 3% methanol in DCM to afford the title compound (D30) as a yellow solid (0.8 g; yield 30%).

MS; (ES) m/z: 246.3 [MH⁺]. C₁₄H₁₁FN₃ requires 245.

¹H-NMR (500 MHz, CDCl₃) δ: 8.30 (d, 1H), 7.32 (dd, 1H), 7.19 (d, 1H), 6.82 (dd, 1H), 3.14 (m, 4H), 3.05 (m, 4H), 2.70 (s, 3H).

### Description 31

### 6-(8-Hydroxy-1,4-dioxaspiro[4.5]dec-8-yl)-4H-benzo[1.4]oxazin-3-one (D31)

A stirred solution of 6-bromo-4H-benzo[1,4]oxazin-3-one (0.5 g, 2.2 mmol, 1.0 eq.) in dry THF (4 mL) was cooled to −30° C. and a 1.6 M solution of n-butyllithium in hexanes (3 mL, 4.8 mmol, 2.2 eq) was added dropwise. The reaction mixture was stirred for 0.5 h then a solution of 1,4-dioxaspiro[4.5]decan-8-one (0.75 g, 4.8 mmol, 2.2 eq) in dry THF (4 mL) was added dropwise. The reaction mixture was stirred at −30° C. for 1 h, then a saturated aq. solution of ammonium chloride (20 mL) was added and the mixture was extracted with ethyl acetate (3×20 mL). The organic layers were combined, dried (Na₂SO₄) and concentrated in vacuo. The crude product was purified by flash chromatography on silica gel, eluting with 50% ethyl acetate in cyclohexane, to give the title compound (D31) as a yellowish solid (0.101 g, yield 15%).

MS; (ES) m/z: 306 [MH⁺]. C₁₆H₁₉NO₅ requires 305.

¹H-NMR (500 MHz, DMSO) δ: 10.59 (s, 1H), 7.04 (d, 1H), 6.96 (dd, 1H), 6.84 (d, 1H), 4.85 (s, 1H), 4.51 (s, 2H), 3.87 (s, 4H), 1.88 (m, 4H), 1.6 (m, 2H), 1.5 (m, 2H).

### Description 32

### 6-(4-Oxocyclohex-1-enyl)-4H-benzo[1,4]oxazin-3-one (D32)

A solution of 6-(8-hydroxy-1,4-dioxaspiro[4.5]dec-8-yl)-4H-benzo[1.4]oxazin-3-one (D31) (84 mg, 0.28 mmol) in trifluoroacetic acid (4 mL) was stirred at r.t. for 1 h. The reaction mixture was concentrated in vacuo, then the residue was dissolved in ethyl acetate (20 mL) and treated with a saturated aq. solution of sodium hydrogencarbonate (20 ml). The organic phase was separated, dried (Na₂SO₄) and concentrated in vacuo to afford the title compound (D32) as a white solid (58 mg, yield 87%).

MS; (ES) m/z: 244 [MH⁺]. C₁₄H₁₃NO₃ requires 243.

¹H-NMR (300 MHz, DMSO) δ: 10.70 (s, 1H), 7.05 (d, 1H), 6.95 (m, 2H), 6.0 (m, 1H), 4.55 (s, 2H), 3.02 (m, 2H), 2.75 (m, 2H), 2.5 (m, 2H).

### Description 33

Benzoic acid 4-methoxycarbonylmethoxy-3-nitrophenyl ester (D33)

A suspension of 4-hydroxyphenyl benzoate (24.7 g, 0.115 mol) in acetic acid (500 mL) was cooled (ice/water bath) and treated dropwise with nitric acid (90%, 7.3 mL, 0.173 mol) over 10 minutes whilst maintaining the temp below 20° C. The mixture was then stirred at r.t. for 16 h. The mixture was reduced in vacuo and the residue triturated with water for 3 h. The resultant yellow solid was filtered off and dried (29.3 g, 98%). A solution of this solid (20.0 g, 0.077 mol) in acetone (400 mL) was treated with potassium carbonate (16.0 g, 0.116 mol) and methyl bromoacetate (11.0 mL, 0.116 mol) and heated at reflux for 16 h. The solvent was removed in vacuo and the residue partitioned between aq. NaHCO₃ (250 mL) and DCM (4×200 mL). The combined organics were dried (Na₂SO₄) and evaporated to a buff solid which was triturated with 40-60° petrol to afford the title compound (D33) as a buff powder (12.7 g, 50%).

MS; (ES) m/z: 332 [MH⁺]. C₁₆H₁₃NO₇ requires 331.

¹H-NMR (300 MHz, CDCl₃) δ: 8.15 (m, 2H), 7.80 (m, 1H), 7.60 (t, 1H), 7.50 (t, 2H), 7.40 (dd, 1H), 7.05 (d, 1H), 4.77 (s, 2H), 3.80 (s, 3H).

### Description 34

### (4-Hydroxy-2-nitrophenoxy)acetic acid methyl ester (D34)

A suspension of benzoic acid 4-methoxycarbonylmethoxy-3-nitrophenyl ester (D33) (10.5 g, 0.03 mol) in methanol (200 mL) was treated dropwise with a solution of sodium methoxide (1.8 g, 0.033 mol) in methanol (100 mL) over 20 minutes. The resulting solution was stirred at r.t. for 3 h, reduced in vacuo to ca. 100 mL and partitioned between water (300 mL) and Et₂O:cyclohexane (1:5, 200 mL). The aqueous phase was separated, acidified (1N HCl to pH 5-6) and extracted with DCM (3×250 mL). The combined organics were dried (Na₂SO₄), evaporated in vacuo and triturated with Et₂O:cyclohexane (1:3, 100 mL) to afford the title compound (D34) as an orange solid 4.35 g, 64%.

¹H-NMR (300 MHz, CDCl₃) δ: 7.33 (d, 1H), 6.97 (m, 2H), 5.35 (br s, 1H), 4.67 (s, 2H), 3.80 (s, 3H).

### Description 35

### [4-(2-Bromoethoxy)-2-nitrophenoxy]acetic acid methyl ester (D35)

A solution of (4-hydroxy-2-nitrophenoxy)acetic acid methyl ester (D34) (0.50 g, 2.20 mmol) in DMF (10 mL) was treated with potassium carbonate (1.5 g, 11.0 mmol) and 1,2-dibromoethane (1.9 mL, 22.0 mmol). The mixture was heated at 85° C. for 6 h, evaporated in vacuo and the residue partitioned between water (50 mL) and DCM (3×50 mL). The combined organics were dried (Na₂SO₄), evaporated in vacuo and the residue chromatographed on silica gel (eluent 10% EtOAc/cyclohexane to 20% EtOAc/cyclohexane) to afford a yellow oil (0.51 g, 69%).

¹H-NMR (300 MHz, CDCl₃) δ: 7.33 (d, 1H), 7.07 (m, 1H), 6.97 (d, 1H), 4.70 (s, 2H), 4.25 (t, 2H), 3.75 (s, 3H), 3.60 (t, 2H).

### Description 36

### 6-(2-Bromoethoxy)-4H-benzo[1,4]oxazin-3-one (D36)

A mixture of [4-(2-bromoethoxy)-2-nitrophenoxy]acetic acid methyl ester (D35) (200 mg, 0.60 mmol) and iron powder (230 mg, 4.20 mmol) in acetic acid (5 mL) was stirred at room temp under nitrogen for 3 h. Water (10 mL) was added, the mixture basified (K₂CO₃) and the resultant dark green solution extracted with DCM (3×30 mL). The combined organic extracts were dried (Na₂SO₄) and evaporated in vacuo to afford the title compound (D36) a white solid (128 mg, 79%).

¹H-NMR (300 MHz, CDCl₃) δ: 8.35 (br s, 1H), 6.88 (d, 1H), 6.50 (d, 1H), 6.40 (s, 1H), 4.55 (s, 2H), 4.20 (t, 2H), 3.55 (t, 2H).

MS; (ES) m/z: 272/274 [MH⁺]. C₁₀H₁₀NO₃ requires 272.

### Description 37

### 2-Chloro-1-(5-chloro-2-fluoro-4-hydroxyphenyl)ethanone (D37)

To a solution of 1-chloro-4-fluoro-2-methoxy-benzene (12.9 g, 80 mmol) in 1,2-dichloroethane (80 mL) at room temperature, chloroacetyl chloride (7.7 mL, 96 mmol) and aluminium trichloride (21.4 g, 0.16 mmol) were added. The solution was heated to 70° C. and stirred at this temperature for 3 h under nitrogen. After cooling to room temperature, the reaction mixture was carefully poured onto crushed ice and extracted with DCM (2×150 mL). Washing of the organic layers with brine (200 mL) followed by drying (Na₂SO₄) and removal of the solvent in vacuo afforded a crude which was purified by flash chromatography eluting with 20% cyclohexane in ethyl acetate. The oil collected (8 g) was a mixture containing the title compound together with 2-chloro-4-(2-chloro-acetyl)-5-fluoro-phenyl chloroacetate. The mixture (7.8 g) was dissolved in methanol (100 mL) and a 2M aqueous solution of sodium carbonate (45 mL) was added. The solution was stirred at room temperature for 1 h then the organic solvent was removed under vacuum and the remaining solution was acidified with a 5% aqueous solution of HCl, extracted with DCM (120 mL), washed with brine (80 mL) and dried (Na₂SO₄). Removal of the solvent in vacuo afforded the title compound (D37) as a brown solid (6.8 g, 38%).

MS; (EI) m/z: 222 [M]⁺. C₈H₅Cl₂FO₂ requires 222.

¹H-NMR (500 MHz, DMSO-d⁶) δ: 11.8 (bs, 1H), 7.87 (d, 1H), 6.83 (d, 1H), 4.94 (d, 2H)

### Description 38

### 2-Chloro-4-(2-chloroethyl)-5-fluorophenol (D38)

A solution of 2-chloro-1-(5-chloro-2-fluoro-4-hydroxyphenyl)ethanone (D37) (5.73 g, 25.7 mmol) in trifluoroacetic acid (25 mL) was cooled to 0° C. and triethylsilane (9.03 mL, 56.5 mmol) was added dropwise. The mixture was allowed to warm to room temperature and then stirred under nitrogen for 2 h. The reaction mixture was concentrated in vacuo diluting repeatedly with ethyl acetate. The crude mixture was slowly poured onto crushed ice and solid sodium carbonate and basified further with a 5% aqueous solution of NaOH. The aqueous layer was separated from the organic layer, cooled at 0° C., acidified with a 10% aqueous solution of HCl and extracted with dichloromethane (150 mL). Drying (Na₂SO₄) and evaporation of the solvent under vacuum afforded the title compound (D38) as a dark brown oil (2.38 g, 42%).

MS; (EI) m/z: 208 [M]⁺. C₈H₅Cl₂FO₂ requires 208.

¹H-NMR (500 MHz, DMSO-d⁶) δ: 10.51 (bs, 1H), 7.33 (d, 1H), 6.70 (d, 1H), 3.73 (t, 2H), 2.90 (t, 2H).

### Description 39

### 6-Chloro-4-(2-chloroethyl)-3-fluoro-2-nitrophenol (D39)

A solution of 2-chloro-4-(2-chloroethyl)-5-fluorophenol (D38) (1.95 g, 9.3 mmol) in glacial acetic acid (10 mL) was cooled to 0° C. and 90% nitric acid (0.48 mmol, 1.03 mmol) was added dropwise. After 45 min of stirring at 0° C. the reaction mixture was poured onto crushed ice, extracted with ethyl acetate (2×100 mL) and the organic layers washed with water (100 mL) and brine (100 mL) and dried (Na₂SO₄). The solvent was removed in vacuo giving a crude brown oil, which was purified by flash chromatography eluting with 33% ethyl acetate in cyclohexane. The title compound (D39) was obtained as an orange solid (1.66 g, 70%).

MS; (EI) m/z: 253 [M]⁺. C₈H₅Cl₂FO₂ requires 253.

¹H-NMR (500 MHz, CDCl₃) δ: 7.6 (d, 1H), 3.7 (t, 2H), 3.10 (td, 2H).

### Description 40

### 2-Amino-4-(2-chloroethyl)-3-fluorophenol (D40)

A mixture of 10% w/w palladium on carbon (0.200 g, 20% w/w) and 6-chloro-4-(2-chloroethyl)-3-fluoro-2-nitrophenol (D39) (1 g, 3.9 mmol) in absolute ethanol (12 mL) was hydrogenated at atomospheric pressure with vigorous stirring for 8 h. A further addition of 10% w/w palladium on carbon (0.1 g, 10% w/w) was made and the stirred suspension was left under a hydrogen atmosphere for another 16 h. The catalyst was filtered off washing the filter with ethanol and the solvent was removed in vacuo affording the title compound (D40) as a white solid (0.75 g, quantitative yield).

MS; (ES) m/z: 190.6 [MH]⁺. C₈H₉ClFO requires 189.

¹H-NMR (500 MHz, DMSO-d⁶) δ: 10.80 (bs, 1H), 6.95 (t, 1H), 6.75 (dd, 1H), 8.0÷6.0 (broad, 2H), 3.75 (t, 2H), 2.95 (t, 2H)

### Description 41

### 6-(2-Chloroethyl)-5-fluoro-4H-benzo[1,4]oxazin-3-one (D41)

A solution of chloroacetyl chloride (0.23 mL, 2.9 mmol) in dry THF (1.5 mL) was added dropwise to a stirred mixture of 2-amino-4-(2-chloroethyl)-3-fluorophenol (D40) (0.500 g, 2.6 mmol) and solid sodium hydrogen carbonate (0.49 g, 5.9 mmol) in dry THF (6 mL) under nitrogen at 0° C. After 30 min. of stirring at 0° C., the solvent was removed under vacuum and the residue was dissolved in butan-2-one (2.5 mL) and water (2.5 mL). Solid potassium carbonate (0.800 g, 5.9 mmol) was added and the reaction mixture was heated at reflux for 1 h. The mixture was then diluted with ethyl acetate (15 mL) and washed with water (15 mL) and brine (15 mL) and dried (Na₂SO₄). Evaporation of the solvent in vacuo gave a crude yellow solid which was purified by flash chromatography eluting with 33% ethyl acetate in cyclohexane affording the title compound (D41) as a white solid (0.320 g, 71%).

MS; (ES) m/z 230.6 [MH]⁺. C₁₀H₉ClFNO₂ requires 229.

¹H-NMR (500 MHz, CDCl₃) δ: 8.00 (bs, 1H), 6.80 (t, 1H), 6.72 (d, 1H), 4.55 (s, 2H), 3.67 (t, 2H), 3.03 (t, 2H).

### Description 42

### 6-(2-Chloroethyl)-5-fluoro-4-methyl-4H-benzo[1,4]oxazin-3-one (D42)

6-(2-Chloroethyl)-5-fluoro-4H-benzo[1,4]oxazin-3-one (D41) (0.1 g, 0.43 mmol) was dissolved in dry DMF (1 mL). The solution was cooled to 0° C. and 60% w/w sodium hydride dispersion in mineral oil (0.019 g, 0.48 mmol) was added under nitrogen. The solution was stirred at that temperature for 10 minutes then methyl iodide (0.041 mL, 0.65 mmol) was added. The reaction was allowed to warm to room temperature and stirred for 30 min. The reaction mixture was diluted with DCM (15 mL), washed with water (2×10 mL) and brine (10 mL) and dried (Na₂SO₄). Removal of the solvent in vacuo afforded a crude which was purified by flash chromatography eluting with 33% ethyl acetate in cyclohexane affording the title compound (D42) as a white solid (0.080 g, 75%).

MS; (ES) m/z 244.6 [MH]⁺. C₂₄H₂₅FN₄O₂ requires 243.

¹H-NMR (500 MHz, CDCl₃) δ: 6.80 (t, 1H), 6.70 (d, 1H) 4.50 (s, 2H), 3.70 (t, 2H), 3.50 (d, 3H), 3.10 (td, 2H).

### Description 43

### 6-(2-Chloroethyl)-4-methyl-4H-benzo[1,4]oxazin-3-one (D43)

To a solution of 6-(2-chloroethyl)-4H-benzo[1,4]oxazin-3-one (D4) (1.0 g, 4.74 mmol) in THF, at 0° C., a 60% w/w suspension of NaH in mineral oil (240 mg, 1.5 eq.) was added in portions. After 40 minutes iodomethane (0.31 mL) was added and the reaction was allowed to reach room temperature and left overnight. The reaction was quenched with a saturated aqueous solution of NH₄Cl, diluted with ethyl acetate, washed with brine, dried (Na₂SO₄) and concentrated under vacuum. The crude was purified by chromatography eluting with 20% ethyl acetate in cyclohexane affording the title compound (D43) as a white solid (840 mg, 79%).

MS; (ES) m/z: 226.6 [MH]⁺. C₁₁H₁₂ClNO₂ requires 225.

¹H-NMR (300 MHz, CDCl₃) δ: 6.85 (d, 1H), 6.78 (dd, 1H), 6.73 (d, 1H), 4.55 (s, 2H), 3.60 (t, 2H), 3.30 (s, 3H), 2.98 (t, 2H).

### Description 44

### 6-(2-Chloroethyl)-4-ethyl-4H-benzo[1,4]oxazin-3-one (D44)

To a solution of 0.5 g of 6-(2-chloroethyl)-4H-benzo[1,4]oxazin-3-one (D4) (2.37 mmol) in THF (15 ml), at 0° C. a 60% w/w suspension of NaH in mineral oil (1190 mg, 1.5 eq.) was added in portions. After 40 minutes of iodomethane (199□L, 1.05 eq.) was added and the reaction was allowed to reach room temperature and then brought to reflux. The reaction was quenched with a saturated aqueous solution of NH₄Cl, diluted with ethyl acetate, washed with brine, dried (Na₂SO₄) and concentrated under vacuum. The crude was purified by chromatography eluting with 20% ethyl acetate in cyclohexane affording the title compound (D44) as a pale yellow solid (400 mg, 71%).

MS; (ES) m/z: 240 [MH]⁺. C₁₂H₁₄ClNO₂ requires 239.

¹H-NMR (300 MHz, CDCl₃) δ: 6.90 (d, 1H), 6.75 (d+s, 2H), 4.55 (s, 2H), 3.95 (q, 2H), 3.50 (t, 2H), 2.90 (t, 2H), 1.05 (t, 3H).

### Description 45

### 1,1-Dimethylethyl(1S,4S)-5-(2-methyl-5-quinolinyl)-2,5-azabicyclo[2.2.1]heptane-2-carboxylate (D45)

1,1-Dimethylethyl(1S,4S)-2,5-diazabicyclo[2.2.1]heptane-2-carboxylate (135 mg; 0.68 mmol; 2 eq.), caesium carbonate (168 mg; 0.515 mmol; 1.5 eq.), palladium acetate (8 mg; 0.034 mmol; 0.1 eq.) and 2,2′-bis(diphenylphosphino)-1,1′-binaphthyl (32 mg; 0.05 mmol; 0.15 eq.) were added to a solution of 2-methylquinolin-5-yl-trifluoromethanesulfonate (D1) (100 mg, 0.34 mmol; 1 eq) in dry toluene (2.5 ml) under nitrogen. The reaction mixture was stirred at reflux under nitrogen for 5 h. The reaction was quenched at r.t. with a saturated aq. solution of ammonium chloride (100 mL) and extracted into ethyl acetate (3×50 ml). The organic layers were combined, dried (Na₂SO₄) and concentrated in vacuo. The crude product was purified by SPE cartridge (Si, 2 g), eluting with a gradient of 2 to 5% of ethyl acetate in cyclohexane to afford the title compound (D45) (37 mg; yield 32%).

MS; (ES) m/z: 340.3 [MH]⁺. C₂₀H₂₅N₃O₂ requires 339.44.

¹H-NMR (300 MHz, CDCl₃) δ: 8.36 (d, 1H), 7.67 (t, 1H), 7.55 (d, 1H), 7.29 (d, 1H), 6.85 (d, 1H), 4.7-4.5 (conf, 1H), 4.35 (bs, 1H), 3.8-3.35 (m, 4H), 2.75 (s, 3H), 2.1 (m, 1H), 1.95 (m, 1H), 1.5 (s, 9H).

### Description 46

### 5-[(1S,4S)-2,5-diazabicyclo[2.2.1]hept-2-yl]-2-methylquinoline (D46)

1,1-Dimethylethyl(1S,4S)-5-(2-methyl-5-quinolinyl)-2,5-azabicyclo[2.2.1]heptane-2-carboxylate (D45) (37 mg; 0.11 mmol; 1 eq) was dissolved in a mixture 3:1 TFA:dry DCM (4 ml) and the solution stirred at RT for 6 h. The solvent was evaporated in vacuo and the residue purified on SCX cartridge (1 g) to give D46 (24 mg; yield 95%).

MS; (ES) m/z: 240.3 [MH]⁺. C₁₅H₁₇N₃ requires 239.44.

¹H-NMR (300 MHz, CDCl₃) δ: 8.36 (d, 1H), 7.55-7.4 (t/d, 2H), 7.15 (d, 1H), 6.83 (dd, 1H), 4.23 (m, 1H), 3.73 (m, 2H), 3.4-3.3 (m, 2H) 3.05 (m, 1H), 2.7 (s, 3H), 2.0 (m, 1H), 1.5 (m, 1H).

### Description 47

### 2-(1-piperazinyl)quinoline (D47)

A mixture of 2-chloroquinoline (2 g; 0.0122 mole; 1 eq) and piperazine (10.5 g; 0.122 mole; 10 eq.) in DMSO (5 ml) was refluxed for 2 h. The reaction mixture was cooled to RT, diluted with H₂O (20 ml) and extracted with DCM (3×20 ml). The organic phases were combined, dried (Na₂SO₄) and concentrated in vacuo. The crude product was purified by SPE cartridge (Si, 10 g), eluting with a gradient of 0 to 1% of MeOH in DCM affording the title compound (1.42 g; yield 55%).

MS; (ES) m/z: 214.3 [MH⁺]. C₁₃H₁₅N₃ requires 213.28.

¹H NMR (300 MHz, CDCl₃) δ: 7.85 (d, 1H), 7.74 (d, 1H), 7.97-7.45 (t/d, 2H), 7.2 (t, 1H), 6.9 (d, 1H), 3.67 (t, 4H), 2.98 (t, 4H).

### Description 48

### 6-(3-Chloropropanoyl)-2H-1,4-benzoxazin-3(4H)-one (D48)

Aluminum chloride (20 g, 147 mmol; 2.2 eq) was added portion-wise to a suspension of 2H-1,4-benzoxazin-3(4H)-one (10 g, 67 mmol; 1.0 eq) in dichloroethane (120 ml) at room temperature. 3-Chloropropionyl chloride (7.5 ml, 80 mmol; 1.2 eq) was then added and the reaction mixture heated at 80° C. for 3 h. The reaction mixture was cooled to room temperature, poured onto ice and extracted with DCM (3×200 ml). The organic layers were combined, dried (Na₂SO₄) and concentrated in vacuo. The crude product was triturated with MeOH affording the title compound (D48) as a brown solid (15 g; yield 93%).

MS; (ES) m/z: 240.5 [MH⁺]. C₁₁H₁₀ClNO₃ requires 239.66.

¹H NMR (300 MHz, DMSO) δ: 10.98 (s, 1H), 7.8 (m, 1H), 7.5 (d, 1H), 7 (m, 1H), 4.5 (s, 2H), 3.9 (t, 2H), 3.45 (t, 2H).

### Description 49

### 6-(3-Chloropropyl)-2H-1,4-benzoxazin-3(4H)-one (D49)

6-(3-Chloropropanoyl)-2H-1,4-benzoxazin-3(4H)-one (D48) was converted to the title compound (D49) following the reduction procedure reported for Description 4.

MS; (ES) m/z: 226.2 [MH⁺]. C₁₁H₁₂ClNO₂ requires 225.67.

¹H NMR (300 MHz, DMSO) δ: 10.5 (s, 1H), 6.8 (d, 1H), 6.75 (m, 2H), 4.5 (s, 2H), 3.5 (t, 2H), 2.5 (t, 2H), 2.0 (q, 2H).

### Description 50

### 2-Formyl-3-methoxy-N-pivaloylaniline (D50)

To a solution of 3-methoxy-N-pivaloylaniline (3 g; 14.5 mmol) in THF (40 ml) under nitrogen at 0° C., n-buthyllithium (22.6 mL solution 1.6 M in hexane; 2.5 eq) was added dropwise and after 1 h dry N,N-dimethylformamide (1.7 mL; 1.5 eq) was added. The reaction was allowed to stir to r.t. overnight, then quenched with a saturated aqueous solution of ammonium chloride (100 mL) and extracted with ethyl acetate (3×50 mL). The organic layers were combined, dried (Na₂SO₄) and concentrated in vacuo. The crude product was purified by flash chromatography, eluting with 10% ethyl acetate in cyclohexane to afford the title compound (D50) as a white solid (2.2 g; yield 65%).

MS; (ES) m/z: 236 [MH⁺]. C₁₃H₁₇NO₃ requires 235.

¹H NMR (300 MHz, CDCl₃) δ: 11.9 (bs, 1H), 10.5 (s, 1H), 8.3 (d, 1H), 7.5 (t, 1H), 6.6 (d, 1H), 3.9 (s, 3H), 1.3 (s, 9H).

### Description 51

### 2,2-Dimethyl-N-{3-(methyloxy)-2-[(1E)-3-oxo-3-phenyl-1-propen-1-yl]phenyl}propanamide (D51)

To a solution of acetophenone (0.1 mL; 0.94 mmol) in dry THF (2 ml) at 0° C. under nitrogen, potassium bis(trimethylsilyl)amide (1.88 ml of a 0.5M solution in toluene; 1 eq) was added dropwise. The solution was allowed to stir to room temperature for 2 h and then it was cooled to −30° C. and a solution of 2-formyl-3-methoxy-N-pivaloylaniline (D50) (200 mg; 0.9 eq) in THF (2 ml) was added dropwise. The reaction mixture was stirred for 1 h, quenched at −30° C. with a saturated aqueous solution of ammonium chloride (20 ml) and then extracted with ethyl acetate (3×20 ml). The organic layers were combined, dried (Na₂SO₄) and concentrated in vacuo. The title compound (D51) was isolated via precipitation with cyclohexane as a white solid (235 mg; 82%).

MS; (ES) m/z: 338.2 [MH⁺]. C₂₁H₂₃NO₃ requires 337.

¹H NMR (500 MHz, DMSO) δ: 9.28 (bs, 1H), 7.96 (d, 2H), 7.82 (d, 1H), 7.69 (d, 1H), 7.64 (t, 1H), 7.55 (t, 2H), 7.38 (t, 1H), 7.03 (d, 1H), 6.85 (d, 1H), 3.95 (s, 3H), 1.17 (s, 9H).

### Description 52

### 5-Methoxy-2-phenylquinoline (D52)

A solution of 2,2-dimethyl-N-{3-(methyloxy)-2-[(1E)-3-oxo-3-phenyl-1-propen-1-yl]phenyl}propanamide (D51) (100 mg; 0.3 mmol) in sulfuric acid (10 ml, 30% in water) was heated to 160° C. with stirring for 1 h. The mixture was cooled, carefully basified (NaHCO₃) and extracted with ethyl acetate (3×15 ml). The combined extracts were dried (Na₂SO₄) and then concentrated in vacuo to obtain the title compound (D52) as a yellow oil (68 mg, 96%) which was used without further purification for the next step.

MS; (ES) m/z: 236.2 [MH⁺]. C₁₆H₁₃NO requires 235.

¹H NMR (300 MHz, CDCl₃) δ: 8.75 (d, 1H), 8.18 (m, 2H), 8.10 (d, 1H), 7.85 (d, 1H), 7.70 (t, 1H), 7.55 (m, 3H), 6.89 (d, 1H), 4.05 (s, 3H).

### Description 53

### 5-Hydroxy-2-phenylquinoline (D53)

A solution of 5-methoxy-2-phenylquinoline (D52) (65 mg; 0.27 mmol) in hydrobromic acid (2 ml, 48% in water) was heated to 130° C. with stirring for 20 h. The mixture was cooled, cautiously basified (NaHCO₃) to pH 7 and extracted with ethyl acetate (3×10 ml). The combined organic layers were dried (Na₂SO₄) and then concentrated in vacuo to obtain the title compound (D4) as a yellow oil (40 mg, 67%). The crude product was used without further purification for the next step.

MS; (ES) m/z: 222.1 [MH⁺]. C₁₁H₁₁NO requires 221.

¹H NMR (300 MHz, DMSO) δ: 8.58 (d, 1H), 8.25 (m, 2H), 8.18 (d, 1H), 7.51 (m, 5H), 6.92 (d, 1H).

### Description 54

### 2-Phenylquinolin-5-yl trifluoromethanesulfonate (D54)

A solution of 5-hydroxy-2-phenylquinoline (D53) (100 mg; 0.45 mmol) and pyridine (0.48 ml; 5 eq) in DCM (25 ml) was cooled to 0° C. and trifluoromethanesulfonic anhydride (0.72 ml; 1.6 eq) was added dropwise. The reaction mixture was stirred under nitrogen at r.t. for 1 h, poured into water (20 ml) and then extracted with ethyl acetate (3×15 mL). The organic layers were combined, dried (Na₂SO₄) and concentrated in vacuo. The crude product was purified by flash chromatography, eluting with 45% ethyl acetate in cyclohexane to afford the title compound (D54) as a yellow solid (73 mg; yield 46%).

MS; (ES) m/z: 354.1 [MH⁺]. C₁₆H₁₀F₃NO₃S requires 353.

¹H NMR (300 MHz, CDCl₃) δ: 8.5 (d, 1H), 8.2 (m, 3H), 8.05 (d, 1H), 7.75 (t, 1H), 7.55 (m, 4H).

### Description 55

### tert-Butyl 4-(2-phenylquinolin-5-yl)piperazine-1-carboxylate (D55)

tert-Butyl-1-piperazinecarboxylate (41 mg; 1.28 eq), caesium carbonate (82 mg; 1.5 eq), palladium acetate (4 mg; 0.1 eq) and 2,2′-bis(diphenylphosphino)-1,1′-binaphthyl (16 mg; 0.15 eq) were added to a solution of 2-phenyl-quinolin-5-yl-trifluoro-methanesulfonate (D54) (60 mg, 0.17 mmol) in dry toluene (2.5 ml) under nitrogen and the reaction mixture was stirred at reflux for 6 h. The reaction was cooled to r.t., quenched with saturated aqueous ammonium chloride (10 ml) and then extracted with ethyl acetate (3×15 mL). The organic layers were combined, dried over anhydrous sodium sulfate and concentrated in vacuo. The crude product was purified by flash chromatography, eluting with 50% ethyl acetate in cyclohexane to afford the title compound (D55) as a yellow oil (64 mg; 96%).

MS; (ES) m/z: 390.3 [MH]⁺. C₂₄H₂₇N₃O₂ requires 389.

¹H-NMR (300 MHz, CDCl₃) δ: 8.6 (d, 1H), 8.2 (m, 2H), 7.8 (m, 2H), 7.6 (t, 1H), 7.5 (m, 3H), 7.1 (d, 1H), 3.7 (bs, 4H), 3.0 (bs, 4H), 1.51 (s, 9H).

### Description 56

### 2-Phenyl-5-piperazin-1-ylquinoline (D56)

tert-Butyl 4-(2-phenyl-quinolin-5-yl)piperazine-1-carboxylate (D55) (62 mg, 0.16 mmol) in a 25% solution of trifluoroacetic acid in DCM (1 ml) was stirred at room temperature under nitrogen for 3 h. The reaction mixture was concentrated in vacuo and filtered through a SCX cartridge to afford the title compound (D56) as a yellow oil (43 mg; 100%).

MS; (ES) m/z: 290.3 [MH]⁺. C₁₉H₁₉N₃ requires 289.

¹H-NMR (300 MHz, CDCl₃) δ: 8.6 (d, 1H), 8.2 (d, 2H), 7.9 (m, 2H), 7.6 (t, 1H), 7.5 (m, 3H), 7.1 (d, 1, H), 3.2 (bs, 4H), 3.0 (bs, 4H).

### Description 57

### Methyl [(2-fluoro-6-nitrophenyl)oxy]acetate (D57)

A mixture of 2-fluoro-6-nitrophenol (5.31 g, 33.8 mmol), methylbromoacetate (3.90 ml, 41.2 mmol) and potassium carbonate (6.59 g, 44.7 mmol) in acetone (130 ml) was stirred at reflux (70° C.) for 5 h. The resulting reaction mixture was concentrated in vacuo, taken-up in ethyl acetate (100 ml), washed with water (2×100 ml) and dried (Na₂SO₄). The solvent was removed in vacuo affording the title compound (D57) as a yellow oil (7.24 g, 93%).

MS; (ES) m/z: 230.20 [MH]⁺. C₉H₈FO₅N requires 229.16.

¹H-NMR (300 MHz, CDCl₃) δ: 7.65 (d, 1H), 7.30 (t, 1H), 7.25-7.15 (m, 1H), 4.85 (s, 2H), 3.80 (s, 3H).

### Description 58

### 8-Fluoro-2H-1,4-benzoxazin-3(4H)-one (D58)

A mixture of methyl [(2-fluoro-6-nitrophenyl)oxy]acetate (D57) (7.24 g, 31.61 mmol) and iron powder (10.60 g, 189.79 mmol) in acetic acid (250 ml) was stirred at room temperature for 14 h under nitrogen. The reaction mixture was concentrated in vacuo, diluted with a saturated aqueous solution of NaHCO₃ (100 ml) and extracted with ethyl acetate (3×100 ml). The combined organic extracts were dried (Na₂SO₄) and evaporated in vacuo to afford the title compound (D58) as a solid (3.89 g, 71%).

¹H-NMR (300 MHz, DMSO-d⁶) δ: 10.85 (bs, 1H), 6.95-6.80 (m, 2H), 6.70 (d, 1H), 4.65 (s, 2H).

### Description 59

### 6-(Chloroacetyl)-8-fluoro-2H-1,4-benzoxazin-3(4H)-one (D59)

To a solution of 8-fluoro-2H-1,4-benzoxazin-3(4H)-one (D58) (3.89 g, 22.4 mmol) in 1,2-dichloroethane (40 ml) at room temperature, chloroacetyl chloride (2.15 ml, 27.0 mmol) and aluminium trichloride (6.57 g, 49.3 mmol) were added. The reaction mixture was stirred at 80° C. for 5 h under nitrogen, concentrated in vacuo, carefully poured into a saturated aqueous solution of NH₄Cl (100 ml) and extracted into ethyl acetate (3×100 ml). The organic layers were washed with brine (200 ml), combined, dried (Na₂SO₄) and concentrated in vacuo. The crude product was purified by flash chromatography on silica gel, eluting with 20% ethyl acetate in cyclohexane to afford the title compound (D59) as a yellow solid (2.70 g, 50%).

¹H-NMR (300 MHz, DMSO-d⁶) δ: 11.10 (s, 1H), 7.62 (d, 1H), 7.31 (s, 1H), 5.11 (s, 2H), 4.76 (s, 2H).

### Description 60

### 6-(2-Chloroethyl)-8-fluoro-2H-1,4-benzoxazin-3(4H)-one (D60)

A solution of 6-(chloroacetyl)-8-fluoro-2H-1,4-benzoxazin-3(4H)-one (D59) (1.32 g, 5.42 mmol) in trifluoroacetic acid (20 ml) was cooled to 0° C. and triethylsilane (2.00 mL, 12.52 mmol) was added dropwise. The reaction mixture was stirred at 0° C. for 10 min under nitrogen, warmed to 45° C. for 20 min and then allowed to stir at room temperature overnight. It was then poured into ice/saturated aqueous solution of NaHCO₃ (20 ml) and extracted into ethyl acetate (3×15 ml). The organic layers were combined, dried (Na₂SO₄) and concentrated in vacuo. The crude product was washed with hexane (30 ml), stirred vigorously for 3 h, filtered and dried (Na₂SO₄) to give the title compound (D60) as a white solid (0.45 g, 37%).

MS; (ES) m/z: 230.20 [MH]⁺. C₁₀H₉ClFNO₂ requires 229.64.

¹H-NMR (300 MHz, DMSO-d⁶) δ: 10.85 (bs, 1H), 6.86 (d, 1H), 6.62 (s, 1H), 4.63 (s, 2H), 3.79 (t, 2H), 2.93 (t, 2H).

### Description 61

### 8-Chloro-5-hydroxy-2-methylquinoline (D61)

A solution of 8-chloro-5-methoxy-2-methylquinoline (WO/0234754) (6.14 g, 25.11 mmol) in 48% hydrobromic acid (120 ml) was stirred at reflux (135° C.) for 20 h. The reaction mixture was concentrated in vacuo, taken-up in a saturated aqueous solution of NaHCO₃ (100 ml) and extracted into ethyl acetate (3×100 ml). The combined organic extracts were dried (Na₂SO₄) and evaporated in vacuo to afford the title compound (D61) as a brown solid (3.79 g, 78%).

MS; (ES) m/z: 194.20 [MH]⁺. C₁₀H₈ClON requires 193.63.

¹H-NMR (500 MHz, DMSO-d⁶) δ: 10.63 (s, 1H), 8.41 (d, 1H), 7.64 (d, 1H), 7.43 (d, 1H), 6.83 (d, 1H), 2.67 (s, 3H).

### Description 62

### 8-Chloro-2-methylquinolin-5-yl trifluoromethanesulfonate (D62)

A solution of 8-chloro-5-hydroxy-2-methylquinoline (D61) (3.79 g, 19.6 mmol) and N,N-diisopropylethylamine (10.40 ml, 59.7 mmol) in dry DCM (50 ml) was cooled to 0° C. and trifluoromethanesulfonic anhydride (5.00 ml, 29.73 mmol) was added dropwise. The reaction mixture was stirred at 0° C. for 2 h under nitrogen, then poured into a saturated aqueous solution of NH₄Cl (50 ml) and extracted into ethyl acetate (3×50 ml). The organic layers were combined, dried (Na₂SO₄) and concentrated in vacuo. The crude product was purified by flash chromatography on silica gel, eluting with 5% ethyl acetate in cyclohexane to afford the title compound (D62) as a yellow solid (2.61 g, 41%).

MS; (ES) m/z: 326.10 [MH⁺]. C₁₁H₇F₃NClO₃S requires 325.69.

¹H-NMR (300 MHz, CDCl₃) δ: 8.27 (d, 1H), 7.84 (d, 1H), 7.49 (d, 1H), 7.31 (d, 1H), 2.86 (s, 3H).

### Description 63

### tert-Butyl 4-(8-chloro-2-methylquinolin-5-yl)piperazine-1-carboxylate (D63)

tert-Butyl 1-piperazinecarboxylate (0.43 g, 2.31 mmol), caesium carbonate (0.89 g, 2.73 mmol), palladium acetate (46.00 mg, 0.20 mmol) and 2,2′-bis(diphenylphosphino)-1,1′-binaphthyl (191.40 mg, 0.31 mmol) were added to a solution of 8-chloro-2-methylquinolin-5-yl trifluoromethanesulfonate (D62) (0.60 g, 1.84 mmol) in dry toluene (30 ml) under nitrogen. The reaction mixture was stirred at 95° C. for 5 h under nitrogen. The reaction was then cooled to room temperature, quenched with a saturated aqueous solution of NH₄Cl (50 ml) and extracted with ethyl acetate (3×50 mL). The organic layers were combined, dried (Na₂SO₄) and concentrated in vacuo. The crude product was purified by flash chromatography on silica gel, eluting with 10% ethyl acetate in cyclohexane to afford the title compound (D63) as a yellow oil (0.43 g, 67%).

MS; (ES) m/z: 362.30 [MH]⁺. C₁₉H₂₄N₃ClO₂ requires 361.87.

¹H-NMR (500 MHz, CDCl₃) δ: 8.40 (d, 1H), 7.70 (d, 1H), 7.30 (d, 1H), 6.95 (d, 1H), 3.70 (bs, 4H), 3.00 (bs, 4H), 2.80 (s, 3H), 1.40 (s, 9H).

### Description 64

### 8-Chloro-2-methyl-5-piperazin-1-ylquinoline (D64)

A solution of tert-butyl 4-(8-chloro-2-methylquinolin-5-yl)piperazine-1-carboxylate (D63) (0.43 g, 1.19 mmol) in trifluoroacetic acid (6 ml) and DCM (2 ml) was stirred at room temperature under nitrogen for 5 h. The reaction mixture was concentrated in vacuo and filtered through a SCX cartridge to afford the title compound (D64) as a yellow solid (0.25 g, 80%).

¹H-NMR (500 MHz, DMSO-d⁶) δ: 8.40 (d, 1H), 7.80 (d, 1H), 7.50 (d, 1H), 7.00 (d, 1H), 2.95 (bs, 4H), 2.90 (bs, 4H), 2.70 (s, 3H).

### Description 65

### Methyl [(2-formyl-6-nitrophenyl)oxy]acetate (D65)

A mixture of 2-hydroxy-5-nitrobenzaldehyde (5.00 g, 29.9 mmol), methylbromo-acetate (6.80 mL, 71.8 mmol) and potassium carbonate (10.0 g, 72.3 mmol) in acetone (50 ml) was stirred at reflux (70° C.) for 14 h. The resulting reaction mixture was filtered and the filtrate concentrated in vacuo. The crude product was purified by flash chromatography on silica gel, eluting with DCM to afford the title compound (D65) as a yellow solid (2.30 g, 38%).

¹H-NMR (300 MHz, CDCl₃) δ: 10.57 (s, 1H), 8.17-8.10 (m, 2H), 7.40 (t, 1H), 5.81 (s, 2H), 3.76 (s, 3H).

### Description 66

### Methyl ({2-[-2-(methyloxy)ethenyl]-6-nitrophenyl}oxy)acetate (D66)

A mixture of methyl [(2-formyl-6-nitrophenyl)oxy]acetate (D65) (1.47 g, 6.11 mmol), (methoxymethyl)-triphenylphosphonium chloride (4.18 g, 12.20 mmol), potassium carbonate (5.48 g, 39.60 mmol) and 18-crown-6 (0.32 g, 1.22 mmol) in dry THF (120 ml) was refluxed (70° C.) for 6 h under nitrogen. The resulting reaction mixture was concentrated in vacuo, taken-up in ethyl acetate (50 ml), washed with a saturated aqueous solution of NH₄Cl (50 ml) and dried over Na₂SO₄. The crude product was purified by flash chromatography on silica gel, eluting with 20% ethyl acetate in cyclohexane to afford the title compound (D66) [mixture of stereoisomers (trans/cis 55/45)] as a pale yellow solid (1.15 g, 70%).

¹H-NMR (300 MHz, CDCl₃) δ: 8.31 (d, 1 Hcis), 7.71-7.59 (m, 1 Htrans+1 Hcis), 7.55 (d, 1 Htrans), 7.26-7.15 (m, 1 Htrans+2 Hcis), 6.38 (d, 1 Hcis), 6.17 (d, 1 Htrans), 5.65 (d, 1 Hcis), 4.69 (s, 2 Hcis +2 Htrans), 3.89 (s, 3 Htrans+6 Hcis), 3.76 (s, 3 Htrans).

### Description 67

### 8-[2-(Methyloxy)ethenyl]-2H-1,4-benzoxazin-3(4H)-one (D67)

A mixture of methyl ({2-[-2-(methyloxy)ethenyl]-6-nitrophenyl}oxy)acetate (D66) (1.15 g, 4.31 mmol), iron powder (1.44 g, 25.82 mmol) and ammonium chloride (2.30 g, 43.2 mmol) in MeOH/H₂O (1/1; 40 ml) was stirred at 80° C. for 7 h. The reaction mixture was cooled to room temperature and filtered through a pad of Celite which was then washed with methanol (30 ml) and DCM (30 ml). The filtrate was concentrated in vacuo, diluted with water (50 ml) and extracted with DCM (3×50 mL). The combined organic extracts were dried (Na₂SO₄) and evaporated in vacuo. The crude product was purified by flash chromatography on silica gel, eluting with 20% ethyl acetate in cyclohexane to afford the title compound (D67) [mixture of stereoisomers (trans/cis 55/45)] as a white solid (0.42 g, 49%).

¹H-NMR (300 MHz, DMSO-d⁶) δ: 10.59 (bs, 1 Htrans+1 Hcis), 7.50 (d, 1 Hcis), 7.29 (d, 1 Htrans), 6.99 (d, 1 Htrans), 6.90-6.80 (m, 1 Htrans+1 Hcis), 6.70-6.60 (m, 1 Htrans+1 Hcis), 6.32 (d, 1 Hcis), 5.89 (d, 1 Htrans), 5.41 (d, 1 Hcis), 4.59 (s, 2 Htrans), 4.54 (s, 2 Hcis), 3.71 (s, 3 Hcis), 3.62 (s, 3 Htrans).

### Description 68

### 4-Methyl-8-[2-(methyloxy)ethenyl]-2H-1,4-benzoxazin-3(4H)-one (D68)

A solution of 8-[2-(methyloxy)ethenyl]-2H-1,4-benzoxazin-3(4H)-one (D67) (0.27 g, 1.31 mmol) and iodomethane (0.10 ml, 1.57 mmol) in dry DMF (25 ml) was cooled to 0° C. and 60% w/w suspension of sodium hydride in mineral oil (73.0 mg, 1.82 mmol) was added in portions under nitrogen. The reaction was allowed to warm to room temperature and stirred for 5 h. The reaction mixture was diluted with ethyl acetate (30 ml), washed with brine (2×30 ml), dried (Na₂SO₄) and concentrated under vacuum. The crude product was purified by flash chromatography on silica gel, eluting with 20% ethyl acetate in cyclohexane to afford the title compound (D68) [mixture of stereoisomers (trans/cis 55/45)] as a yellow oil (178.70 mg, 81%).

¹H-NMR (300 MHz, DMSO-d⁶) δ: 7.61 (d, 1 Hcis), 7.32 (d, 1 Htrans), 7.12 (d, 1 Htrans), 7.01-6.88 (m, 2 Htrans+2 Hcis), 6.38 (d, 1 Hcis), 5.93 (d, 1 Htrans), 4.66 (s, 2 Htrans), 4.60 (s, 2 Hcis), 3.76 (s, 3 Hcis), 3.62 (s, 3 Htrans), 3.24 (s, 3 Htrans+3 Hcis).

### Description 69

### (4-Methyl-3-oxo-3,4-dihydro-2H-1,4-benzoxazin-8-yl)acetaldehyde (D69)

To a solution of 4-methyl-8-[2-(methyloxy)ethenyl]-2H-1,4-benzoxazin-3(4H)-one (D68) (179 mg, 0.81 mmol) in THF (4 ml), 10% hydrochloric acid (12 ml) was slowly added and the reaction was stirred at room temperature for 4 h. The resulting reaction mixture was concentrated in vacuo, taken-up in ethyl acetate (15 ml), washed with water (2×15 ml) and dried over Na₂SO₄. The crude product was purified by SPE cartridge (Si), eluting with 10% ethylacetate in cyclohexane to afford the title compound (D69) as a solid (84 mg, 50%).

¹H-NMR (300 MHz, DMSO-d⁶) δ: 9.68 (s, 1H), 7.17-7.05 (m, 2H), 6.92 (d, 1H), 4.62 (s, 2H), 3.72 (s, 2H), 3.28 (s, 3H).

### Description 70

### (3-Oxo-3,4-dihydro-2H-1,4-benzoxazin-8-yl)acetaldehyde (D70)

The title compound (D70) was prepared in a similar fashion to Description 69 starting from 8-[2-(methyloxy)ethenyl]-2H-1,4-benzoxazin-3(4H)-one (D67) (37 mg, 0.18 mmol) as a solid (23 mg, 67%).

¹H-NMR (300 MHz, CDCl₃) δ: 9.66 (t, 1H), 6.88-6.74 (m, 2H), 4.62 (s, 2H), 3.72 (s, 2H).

### Description 71

### 2-Methyl-5-[(3R)-3-methyl-1-piperazinyl]quinoline (D71)

A mixture of 2-methylquinolin-5-yltrifluoromethanesulfonate (D1) (0.80 g, 2.75 mmol), (R)-2-methylpiperazine (0.27 g, 2.70 mmol), caesium carbonate (1.78 g, 5.49 mmol), palladium acetate (0.12 g, 0.55 mmol) and 2,2′-bis(diphenylphosphino)-1,1′-binaphthyl (0.51 g, 0.82 mmol) in dry toluene (30 ml) was stirred at reflux under nitrogen for 1 h. A further addition of (R)-2-methylpiperazine (0.27 g, 2.70 mmol) was then performed, followed by heating at reflux for 2 h. The reaction was cooled to room temperature, quenched with a saturated aqueous solution of NH₄Cl (50 ml) and extracted with ethyl acetate (3×50 ml). The organic layers were combined, dried (Na₂SO₄) and concentrated in vacuo. The crude product was purified by flash chromatography on silica gel, eluting with 10% methanol in dichloromethane to afford the title compound (D71) as a brown solid (0.19 g, 29%).

MS; (ES) m/z: 242.30 [MH]⁺. C₁₅H₁₉N₃ requires 241.34.

¹H-NMR (300 MHz, CDCl₃) δ: 8.35 (d, 1H), 7.66 (d, 1H), 7.57 (t, 1H), 7.23 (d, 1H), 7.05 (d, 1H), 3.30-3.10 (m, 5H), 2.82 (t, 1H), 2.76 (s, 3H), 2.51 (t, 1H), 1.11 (d, 3H).

### Description 72

### 2-Methyl-5-[(3S)-3-methyl-1-piperazinyl]quinoline (D72)

The title compound (D72) was prepared in a similar fashion to Description 71 starting from 2-methylquinolin-5-yl trifluoromethanesulfonate (D1) (0.80 g, 2.75 mmol) and (S)-2-methylpiperazine (0.27 g, 2.70 mmol) as a brown solid (89.11 mg, 13%).

MS; (ES) m/z: 242.30 [MH]⁺. C₁₅H₁₉N₃ requires 241.34.

¹H-NMR (300 MHz, CDCl₃) δ: 8.35 (d, 1H), 7.66 (d, 1H), 7.57 (t, 1H), 7.23 (d, 1H), 7.05 (d, 1H), 3.30-3.10 (m, 5H), 2.82 (t, 1H), 2.76 (s, 3H), 2.51 (t, 1H), 1.11 (d, 3H).

### Description 73

### 2,2-Dimethyl-2,3-dihydro-1-benzofuran-7-ylmethanesulfonate (D73)

A solution of 7-hydroxy-2,2-dimethyl-2,3-dihydro-1-benzofuran (1 g; 6 mmol) in dry DCM (10 ml) and pyridine (1.45 ml; 3 eq) was cooled to 0° C. and trifluoromethanesulfonic anhydride (1.5 ml; 1.5 eq) was added dropwise. The reaction mixture was stirred under nitrogen at r.t. for 1 h, then poured into saturated aqueous NH₄Cl (75 ml) and extracted three times into DCM (3×75 ml). The organic layers were combined, dried (Na₂SO₄) and concentrated in vacuo. The excess of pyridine was removed by adding toluene and evaporating in vacuo. The crude product (D73) was used without further purification for the next step.

MS; (ES) m/z: 297 [MH⁺]. C11H11F3O4S requires 296.

### Description 74

### tert-Butyl 4-(2,2-dimethyl-2,3-dihydro-1-benzofuran-7-yl)piperazine-1-carboxylate (D74)

tert-Butyl-1-piperazinecarboxylate (1.34 g; 1.2 eq.), caesium carbonate (2.93 g; 1.5 eq.), palladium acetate (188 mg; 0.14 eq.) and 2,2′-bis(diphenylphosphino)-1,1′-binaphthyl (785 mg; 0.21 eq.) were added to a solution of 2,2-dimethyl-2,3-dihydro-1-benzofuran-7-ylmethanesulfonate (D73) (6 mmol) in dry toluene (10 mL) under nitrogen. The reaction mixture was stirred at reflux under nitrogen for 6 h. The reaction was then cooled to r.t. and quenched with saturated aqueous ammonium chloride (50 ml) and then extracted with ethyl acetate (3×50 ml). The organic layers were combined, dried (Na₂SO₄) and concentrated in vacuo. The crude product was purified by flash chromatography, eluting with 50% ethyl acetate in cyclohexane to afford the title compound (D74) as a yellow oil (1.45 g; 73%).

MS; (ES) m/z: 333 [MH]⁺. C₂₄H₂₇N₃O₂ requires 332.

¹H-NMR (300 MHz, CDCl₃) δ: 6.6/6.9 (m, 3H), 3.6 (m, 4H), 3.1 (m, 1H), 3.0 (m, 3H), 2.95 (s, 2H), 1.45 (bs, 15H).

### Description 75

### 2,2-Dimethyl-2,3-dihydro-1-benzofuran-7-yl)piperazine (D75)

tert-Butyl 4-(2,2-dimethyl-2,3-dihydro-1-benzofuran-7-yl)piperazine-1-carboxylate (D74) (500 mg, 1.5 mmol) in a 25% solution of trifluoroacetic acid in DCM (8 ml) was stirred at r.t. under nitrogen for 3 h. The reaction mixture was concentrated in vacuo and filtered through a SCX cartridge to afford the title compound (D75) as a yellow oil (256 mg; 73%).

MS; (ES) m/z: 233 [MH]⁺. C₁₄H₂₀N₂₀ requires 232.

### Method A

### Description 76

### 7-Nitro-2,3-dihydro-1,4-benzodioxin-5-carboxylic acid (D76)

A solution of fuming nitric acid (4 ml) in acetic acid (4 ml) was added drop-wise to a suspension of 2,3-dihydro-benzo[1,4]dioxine-5-carboxylic acid (3.9 g, 21.6 mmol) in acetic acid (20 ml) and acetic anhydride (20 ml) at room temperature. The internal temperature was maintained below 65° C. during the addition. The reaction was stirred at room temperature for 1 h. The solid formed was filtered, washed with water and dried to give 1.4 g of the title compound (D76) as a yellow powder.

¹H-NMR (DMSO, 343 K) δ: 13.33 (s br, 1H); 8.09 (d, 1H); 7.85 (d, 1H); 4.46 (m, 2H); 4.38 (m, 2H).

### Description 77

### 7-Amino-2,3-dihydro-1,4-benzodioxin-5-carboxylic acid hydrochloride (D77)

7-Nitro-2,3-dihydro-1,4-benzodioxin-5-carboxylic acid (D76) (5.5 g, 24.4 mmol) was dissolved in 0.5 N NaOH (50 ml). This solution was hydrogenated over 10% Pd/C (0.9 g) for 16 h at 40 psi. The reaction was filtered, acidified with 20% HCl. The precipitate was filtered-off and dried under vacuum to yield 5.65 g of the title compound (D77) (yield 100%).

¹H-NMR (DMSO, 343 K) δ: 10.40 (s br, 3H); 7.25 (d, 1H); 7.07 (d, 1H); 4.30 (s br, 4H).

### Description 78

### 7-Chloro-2,3-dihydro-1,4-benzodioxin-5-carboxylic acid (D78)

To a solution of 7-amino-2,3-dihydro-1,4-benzodioxin-5-carboxylic acid hydrochloride (D77) (3.4 g, 14.69 mmol) in H₂O (15 ml) and concentrated HCl (4 ml), cooled to −5° C., a solution of NaNO₂ (1.09 g, 15.77 mmol) in H₂O (7 mL) was added drop-wise. The brown solution of the diazionium salt was then added dropwise to a solution of CuCl (1.48 g, 14.69 mmol) in concentrated HCl (5 ml) maintaining the internal temperature around 10° C. The mixture was then diluted with H₂O (120 ml) and the solution was stirred for additional 1 h at room temperature.

The mixture was filtered, the residue washed with water and dried under vacuum to obtain 3.2 g of the title compound (D78) as a yellow solid (yield 100%), m.p.=190-191° C.

¹H-NMR (CDCl₃, 343 K) δ: 7.65 (d, 1H); 7.10 (d, 1H); 4.48 (m, 2H); 4.36 (m, 2H).

### Description 79

### 7-Chloro-2,3-dihydro-1,4-benzodioxin-5-amine (D79)

To a solution of 7-chloro-2,3-dihydro-1,4-benzodioxin-5-carboxylic acid (D78) (3.2 g, 14.9 mmol) in trifluoroacetic acid (40 ml) and trifluoroacetic anhydride (15 ml), NaN₃ (1.94 g, 29.8 mmol) was added at 0° C. The reaction was stirred 5 h at room temperature. The organic phase was removed under vacuum and the residue was dissolved in EtOAc, washed with saturated solution of NaHCO₃, dried over Na₂SO₄, filtered and concentrated to dryness to obtain 4 g of crude N-(7-chloro-2,3-dihydro-benzo[1,4]dioxin-5-yl)-2,2,2-trifluoro-acetamide that was used in the next step.

N-(7-Chloro-2,3-dihydro-benzo[1,4]dioxin-5-yl)-2,2,2-trifluoro-acetamide (4 g) dissolved in MeOH (40 ml) and 2N NaOH (20 ml) was refluxed for 5 h. The organic solvent was removed under vacuum and the residue was dissolved in EtOAc, washed with brine, dried over Na₂SO₄, filtered and concentrated to dryness. The residue was dissolved in 2N HCl, washed with Et2O. The aqueous phase was basified with 2N NaOH, extracted with EtOAc, washed with brine, dried over Na₂SO₄, filtered and concentrated to dryness to obtain 0.6 g of the title compound (D79) as a brown oil (yield=22%).

¹H-NMR (CDCl₃, 343 K) δ: 6.32 (d, 1H); 6.29 (d, 1H); 4.28-4.21 (m, 4H).

### Method B

### Description 80

### 5-Chloro-3-nitro-1,2-benzenediol (D80)

Concentrated H₂SO₄ (6.4 ml) was added to a solution of 35% hydrogen peroxide (307 mmol) in dioxane (110 ml) and the mixture was heated at 40° C. for 1 hour. 4-Chloro-2-hydroxy-3-nitroacetophenone (8 g, 371 mmol) was then added in one portion. Stirring was continued for 15 min. before adding H₃BO₃ (18.9 g, 307 mmol) in one portion. The temperature raised to 80° C. and was maintained for 8 h. The solvent was removed under vacuum and the residue was treated with water (150 ml) and filtered. The solid was washed with water and dried under vacuum. It was then re-dissolved in diethyl ether and the insoluble part was filtered off. The solvent was removed under vacuum to give 5.4 g of the title compound (D80) as an orange powder (yield=77%), m.p. 116-119° C.

¹H-NMR (CDCl₃, 343 K) δ: 10.52 (s, 1H); 7.65 (d, 1 h); 7.23 (d, 1H); 5.97 (s br, 1H); 3.70 (s 4H).

### Description 81

### 7-Chloro-5-nitro-2,3-dihydro-1,4-benzodioxin (D81)

A mixture of 5-chloro-3-nitro-1,2-benzenediol (D80) (5.4 g, 28.5 mmol) anhydrous K₂CO₃ (115.7 mmol), DMF (50 ml) and 1,2-dibromoethane (13.4 g, 6.15 ml, 71.27 mmol) were heated to 80° C. for 5 hours. After cooling to room temperature, water (200 ml) was added and the solution extracted with ethyl acetate. The organic layer was washed with brine, dried over Na₂SO₄, filtered and evaporated under vacuum to yield 4.35 g of the title compound (D81) as a light brown powder (yield=71%) m.p. 135-137° C.

¹H-NMR (CDCl₃, 343 K) δ: 7.49 (d, 1H); 7.10 (d, 1H); 4.42-4.33 (m, 4H).

### Description 79

### 7-Chloro-2,3-dihydro-1,4-benzodioxin-5-amine (D79)

Iron powder (0.85 g, 15.2 mmol) was added to a mixture of 7-chloro-5-nitro-2,3-dihydro-1,4-benzodioxin (D81) (0.8 g, 3.72 mmol) in 96% EtOH (15 ml) and glacial acetic acid (8 ml). The reaction was stirred for 6 hours at room temperature. The reaction mixture was neutralized with saturated solution of NaHCO₃ and then extracted with ethyl acetate. The organic layer was washed with brine, dried over Na₂SO₄ and evaporated to dryness. The residue was treated with 10% HCl and the solution was washed with Et₂O, basified with 2N NaOH, extracted with ethyl acetate, dried and evaporated under vacuum to give 0.35 g of the title compound (D79) as a brown oil (yield=51%).

¹H-NMR (CDCl₃, 343 K) δ: 6.32 (d, 1H); 6.29 (d, 1H); 4.28-4.21 (m, 4H).

### Description 82

### 1-(7-Chloro-2,3-dihydro-1,4-benzodioxin-5-yl)-4-tertbutoxycarbonyl piperazine (D82)

To a solution of 7-chloro-2,3-dihydro-1,4-benzodioxin-5-amine (D79) (0.85 g, 4.58 mmol) in AcOEt (30 ml), finely ground bis-(2-chloroethyl)-amine hydrochloride (1.8 g, 10 mmol) was added, followed by basic alumina (13 g). This suspension was cautiously evaporated on a rotary evaporator, and the resultant dry powder was heated in a glass oven at 160° C. for 2 hours. After cooling, the reaction mixture was suspended in methanol (50 ml), filtered on a sintered funnel and extensively washed with methanol. The filtrate was evaporated and the residue suspended in THF (40 ml). To this suspension an excess of BOC₂O was added (2 g), followed by NaOH 1M (30 ml). After vigorous stirring (15 min) the two phases were separated, the aqueous layer washed with AcOEt and the organic phases dried and evaporated. The residue was purified by silica chromatography eluting with a gradient of petroleum ether/AcOEt, 9/1 to 4/1 to afford the title compound (D82) (1.06 g) as a colourless solid (yield=62%).

ESI POS; AQA: spray 3.5 KV/source 30 V/probe 250° C.: 355.2 (MH+)

### Description 83

### 1-(7-Chloro-2,3-dihydro-1,4-benzodioxin-5-yl)piperazine hydrochloride (D83)

1-(7-Chloro-2,3-dihydro-1,4-benzodioxin-5-yl)-4-tertbutoxycarbonylpiperazine (D82) (1.06 g, 2.99 mmol) was dissolved in methanol (15 ml) and an excess of Et₂O.HCl (10%, 15 ml) was added. The solution was stirred at room temperature for 1 hour, and then evaporated to dryness to yield the title compound (D83) (0.87 g).

¹H-NMR (DMSO, 343 K) δ: 9.24 (s br, 2H); 6.63 (d, 1H); 6.51 (d, 1H); 4.24 (m, 4H); 3.20 (m, 8H).

ESI POS; AQA: spray 3.5 KV/source 30 V/probe 250° C.: 254.9 (MH+)

### Description 84

### 5-Fluoro-3-nitro-1,2-benzenediol (D84)

The title compound (D84) (0.85 g) was prepared according to the method described in Description 80 starting from 1-(5-fluoro-2-hydroxy-3-nitrophenyl)ethanone (1.1 g, 5.5 mmol) in 89% yield.

¹H-NMR (CDCl₃, 343 K) δ: 10.41 (s, 1H); 7.35 (dd, 1H); 7.03 (dd, 1H); 6.27 (s br, 1H).

EI; TSQ 700; source 180° C.; 70 V; 200 uA: (M+) 173; 125.

### Description 85

### 7-Fluoro-5-nitro-2,3-dihydro-1,4-benzodioxin (D85)

The title compound (D85) (0.6 g) was prepared according to the method described for Description 81 starting from 5-fluoro-3-nitro-1,2-benzenediol (D84) (0.85 g, 4.7 mmol) in 72% yield.

¹H-NMR (CDCl₃, 343 K) δ: 7.24 (dd, 1H); 6.88 (dd, 1H); 4.41-4.34 (m, 4H).

### Description 86

### 7-Fluoro-2,3-dihydro-1,4-benzodioxin-5-amine (D86)

A solution of 7-fluoro-5-nitro-2,3-dihydro-1,4-benzodioxin (D85) (0.6 g, 3 mmol) in MeOH (50 ml) was hydrogenated over 10% Pd/C (60 mg) at 45 psi for 24 h. The suspension was filtered and the solvent evaporated to dryness to yield 0.5 g of the title compound (D86) as a brown oil (yield=100%).

ESI POS; AQA: spray 3.5 KV/source 30 V/probe 250° C.: 170.2 (MH+)

### Description 87

### 1-(7-Fluoro-2,3-dihydro-1,4-benzodioxin-5-yl)-4-tertbutoxycarbonylpiperazine (D87)

The title compound (D87) (0.5 g) was prepared according to the method described in Description 81 starting from 7-fluoro-2,3-dihydro-1,4-benzodioxin-5-amine (D86) (0.5 g, 2.95 mmol) in 50% yield.

¹H-NMR (CDCl₃, 343 K) δ: 6.32 (dd, 1H); 6.23 (dd, 1H); 4.31-4.22 (m, 4H); 3.58 (m, 4H); 2.97 (m, 4H); 1.48 (s, 9H).

### Description 88

### 1-(7-Fluoro-2,3-dihydro-1,4-benzodioxin-5-yl)piperazine hydrochloride (D88)

The title compound (D88) was prepared according to the method described for Description 82 starting from 1-(7-fluoro-2,3-dihydro-1,4-benzodioxin-5-yl)-4-tertbutoxycarbonylpiperazine (D87) in 100% yield.

ESI POS; AQA: spray 3.5 KV/source 30 V/probe 250° C.: 239.2 (MH+)

### Description 89

### 5-Bromo-3-nitro-1,2-benzenediol (D89)

The title compound (D89) (3.2 g) was prepared according to general method of Description 80 starting from 1-(5-bromo-2-hydroxy-3-nitrophenyl)ethanone (5 g, 19.2 mmol) in 71% yield, mp=102-105° C.

¹H-NMR (CDCl₃, 343 K) δ: 10.54 (s, 1H); 7.80 (d, 1H); 7.36 (d, 1H); 5.94 (s br, 1H).

### Description 90

### 7-Bromo-5-nitro-2,3-dihydro-1,4-benzodioxin (D90)

The title compound (D90) (3 g) was prepared according to the general method of Description 81 starting from 5-bromo-3-nitro-1,2-benzenediol (D89) (3.4 g, 13.67 mmol) in 95% yield.

¹H-NMR (CDCl₃, 343 K) δ: 7.62 (d, 1H); 7.25 (d, 1H); 4.41-4.33 (m, 4H).

EI; TSQ 700; source 180° C.; 70 V; 200 uA: 259 (M+), 213, 185, 157.

### Description 91

### 7-Bromo-2,3-dihydro-1,4-benzodioxin-5-amine (D91)

The title compound (D91) (1.2 g) was prepared according to the method described for Description 79 starting from 7-bromo-5-nitro-2,3-dihydro-1,4-benzodioxin (D90) (3.2 g, 12.3 mmol) in 42% yield.

ESI POS; AQA: spray 3.5 KV/source 30 V/probe 250° C.: 230.1 (MH+)

### Description 92

### 1-(7-Bromo-2,3-dihydro-1,4-benzodioxin-5-yl)-4-terbutoxycarbonylpiperazine (D92)

The title compound (D92) (1.4 g) was prepared as described for Description 82 starting from 7-bromo-2,3-dihydro-1,4-benzodioxin-5-amine (D91) (1.2, 5.2 mmol).

¹H-NMR (CDCl₃, 343 K) δ: 6.76 (d, 1H); 6.60 (d, 1H); 4.29 (m, 2H); 4.23 (m, 2H); 3.58 (m, 4H); 2.97 (m, 4H); 1.48 (s, 9H).

### Description 93

### 1-(7-Bromo-2,3-dihydro-1,4-benzodioxin-5-yl)piperazine hydrochloride (D93)

The title compound (D93) was prepared according to the method described in Description 83 starting from 1-(7-bromo-2,3-dihydro-1,4-benzodioxin-5-yl)-4-terbutoxycarbonyl-piperazine (D92) in 100% yield.

ESI POS; AQA: spray 3.5 KV/source 30 V/probe 250° C.: 299.1 (MH⁺).

### Description 94

### 6-(Chloroacetyl)-4-methyl-2H-1,4-benzoxazin-3(4H)-one (D94)

To a stirred solution of 4-methyl-2H-1,4-benzoxazin-3(4H)-one (5 g, 30.6 mmol) in DCM (50 ml) at 0° C. solid AlCl₃ (8.16 g, 61.2 mmol) was added portionwise over 15 min. Chloroacetylchloride (2.65 ml, 33.6 mmol) was then added dropwise over 5 min and the resulting mixture was heated at reflux for 3 h. Heating was interrupted, DCM (100 ml) was added and the solution was cooled to 0° C. Crushed ice was added and solid sodium bicarbonate was carefully added until the mixture was neutralised. The organic layer was separated from the aqueous and washed with brine, dried (Na₂SO₄) and the solvent was evaporated in vacuo. The pale brown solid obtained was triturated in Et₂O to afford the title compound (D94) as a pale yellow solid (6.2 g, 85%) after filtration and drying.

MS: (ES/+) m/z: 240 [MH⁺]. C₁₁H₁₀ClNO₃ requires 239.

¹H-NMR (300 MHz, CDCl₃) δ(ppm): 7.6 (m, 2H), 7.0 (d, 1H), 4.65 (2H), 4.55 (2H), 3.35 (s, 3H).

### Description 95

### 8-Fluoro-2-methyl-5-quinolinyl trifluoromethanesulfonate (D95)

A solution of 8-fluoro-2-methyl-quinolin-5-ol (WO/2002034754) (103 mg, 0.58 mmol) and pyridine (1 ml) in DCM (4 ml) was cooled to 0° C. and trifluoromethanesulfonic anhydride (144 μl) was added. The reaction mixture was stirred under an inert atmosphere at r.t., then poured into water and extracted into ethyl acetate (3×). The organic layers were combined, dried (Na₂SO₄) and concentrated under reduced pressure. The crude product was purified by flash chromatography, eluting with ethyl acetate/cyclohexane (2/3) affording the title compound (D95) (134 mg, 74% yield).

¹H-NMR (300 MHz, d₆-DMSO) δ(ppm): 8.31 (m, 1H), 7.85 (m, 1H), 7.60 (m, 2H), 2.82 (s, 3H).

### Description 96

### 4-(8-Fluoro-2-methyl-5-quinolin-5-yl)-piperazine-1-carboxylic acid tert-butyl ester (D96)

1,1-Dimethylethyl-1-piperazinecarboxylate (96 mg, 0.52 mmol), caesium carbonate (211 mg, 0.65 mmol), palladium acetate (14 mg, 0.06 mmol) and 2,2′-bis(diphenylphosphino)-1,1′-binaphthyl (80 mg, 0.13 mmol) were added to a solution of 8-fluoro-2-methyl-5-quinolinyl trifluoromethanesulfonate (D95) (134 mg, 0.43 mmol) in toluene (1.5 ml) under an inert atmosphere. The reaction mixture was stirred at reflux for 6 hours. The reaction was then quenched at room temperature using a saturated aqueous solution of ammonium chloride and extracted into ethyl acetate (3×). The organic layers were combined, dried (Na₂SO₄) and concentrated under reduced pressure. The crude was purified by flash chromatography, eluting with ethyl acetate/cyclohexane (1/9) affording the title compound (D96) (50 mg, 34% yield). MS; (ES) m/z: 346 [MH⁺]. C₁₉H₁₄FN₃O₂ requires 345.

### Description 97

### 8-Fluoro-2-methyl-5-piperazin-1-yl-quinoline (D97)

4-(8-Fluoro-2-methyl-5-quinolinil-5-yl)-piperazine-1-carboxylic acid tert-butyl ester (D96) (50 mg, 0.14 mmol) was dissolved in 1,4-dioxane (0.5 ml) and HCl (2.5 ml of a 4N solution in dioxane) was added under stirring. After stirring for 4 hours, the solvent was evaporated to yield a white solid that was dissolved in water, basified with solid NaOH (pH>10) and extracted with DCM. The organic layer was dried (Na₂SO₄) and then evaporated under reduced pressure to afford the title compound (D97) (50 mg, 70% yield).

MS; (ES) m/z: 246 [MH⁺]. C₁₄H₁₆FN₃ requires 245.

### Description 98

### 5-Bromo-6-fluoro-2-methylquinoline (D98)

To a solution of 6-fluoro-2-methylquinoline (0.50 g, 3.1 mmol) in 1,2-dichloroethane, at 0° C., was added AlCl₃ (0.85 g, 6.4 mmol) and then bromine (550 mg, 3.4 mmol). The mixture was heated to 60° C. for 4 hours, then allowed to reach r.t., and cautiously quenched with water. The solution was basified with solid NaOH (pH>10), extracted with EtOAc, washed with brine and dried (Na₂SO₄). The crude was then purified by silica SPE cartridge eluting with a 9/1 mixture of cyclohexane/ethyl acetate to afford the title compound (D98) as a white solid (596 mg, 80% yield).

¹H-NMR (300 MHz, CDCl₃) δ(ppm): 8.28 (m, 1H), 7.87 (m, 1H), 7.3-7.03 (m, 2H), 2.51 (s, 3H).

### Description 99

### 4-(6-Fluoro-2-methyl-5-quinolin-5-yl)piperazine-1-carboxylic acid tert-butyl ester (D99)

1,1-Dimethylethyl-1-piperazinecarboxylate (186 mg, 1.0 mmol), caesium carbonate (400 mg, 1.2 mmol), palladium acetate (28 mg, 0.12 mmol) and 2,2′-bis(diphenylphosphino)-1,1′-binaphthyl (155 mg, 0.25 mmol) were added to a solution of 5-bromo-6-fluoro-2-methylquinoline (D99) (200 mg, 0.83 mmol) in toluene (2.5 ml) under an inert atmosphere. The reaction mixture was stirred at reflux for 10 hours. The reaction was then quenched at room temperature using a saturated aqueous solution of ammonium chloride and extracted into ethyl acetate. The organic layers were combined, dried (Na₂SO₄) and concentrated under reduced pressure. The crude was purified by flash chromatography, eluting with ethyl acetate/cyclohexane (3/7) affording the title compound (D99) as a green solid (209 mg, 73% yield).

¹H-NMR (300 MHz, CDCl₃) δ(ppm): 8.6 (d, 1H), 7.80 (m, 1H), 7.20 (m, 1H), 7.15 (m, 1H), 4.15 (bs, 2H), 3.3-3.0 (bm, 6H), 2.71 (s, 3H).

### Description 100

### 6-Fluoro-2-methyl-5-(1-piperazinyl)quinoline (D100)

4-(6-Fluoro-2-methyl-5-quinolin-5-yl)piperazine-1-carboxylic acid tert-butyl ester (D99) (209 mg, 0.61 mmol) was dissolved in 1,4-dioxane (2 ml) and HCl (6 ml of a 4N solution in dioxane) was added under stirring. After stirring for 2 hours, the solvent was evaporated to yield a white solid that was dissolved in water, basified with solid NaOH (pH>10) and extracted with DCM. The organic layer was dried (Na₂SO₄) and then evaporated under reduced pressure to afford the title compound (D100) (97 mg, 66% yield).

MS; (ES) m/z: 246 [MH⁺]. C₁₄H₁₆FN₃ requires 245.

### Description 101

### 7-Fluoro-2-methyl-5-quinolinyl trifluoromethanesulfonate (D101)

A solution of 7-fluoro-2-methyl-5-quinolinol (1.67 g; 9.4 mmol) (WO/0234754) in dry DCM (20 ml) and dry diisopropylethylamine (4.9 ml; 28.2 mmol, 3 eq) was cooled to 0° C. and trifluoromethanesulfonic anhydride (2.4 ml, 14.1 mmol, 1.5 eq) was added dropwise over 10 minutes. The reaction mixture was stirred under nitrogen at r.t. for 1 h, then poured into a saturated aqueous ammonium chloride (20 ml) and extracted into ethyl acetate (3×15 ml). The organic layers were combined, dried (Na₂SO₄) and concentrated in vacuo. The crude product was purified by flash chromatography, eluting with 20% ethyl acetate in cyclohexane to afford the title compound (D101) as a pale yellow solid (2.15 g; 73%).

MS; (ES) m/z: 310.1.3 [MH⁺]. C₁₁H₇F₄NO₃S requires 309.

### Description 102

### 4-(7-Fluoro-2-methylquinolin-5-yl)-3,6-dihydro-2H-pyridine-1-carboxylic acid tert-butyl ester (D102)

A mixture of 1(2H)-pyridinecarboxylic acid, 3,6-dihydro-4-(4,4,5,5-tetramethyl-1,3,2 dioxaborolan-2-yl)-1,1-dimethylethyl ester (Tetrahedron Letters 2000, 41, 3705-3708) (0.85 g, 2.75 mmol), 7-fluoro-2-methyl-5-quinolinyl trifluoromethanesulfonate (D101) (0.85 g, 2.7 mmol), [1,1′-bis(diphenylphosphino)ferrocene]palladium (II) chloride (0.221 g, 0.27 mmol) and potassium carbonate (1.2 g, 8.1 mmol) in dry DMF (20 ml) was heated at 80° C. under nitrogen for 3 h. The DMF was removed in vacuo and the residue partitioned between water (25 ml) and DCM (3×50 ml). The organic extracts were dried (Na₂SO₄) and chromatographed on silica (eluent 30% EtOAc/cyclohexane) to afford the title compound (D102) as a colourless oil (0.61 g, 66%).

MS; (ES) m/z: 343.3 [MH⁺]. C₂₀H₂₃N₂O₂F requires 342.

¹H-NMR (300 MHz, CDCl₃) δ: 8.15 (d, 1H), 7.55 (d, 1H), 7.25 (m, 1H), 7.0 (m, 1H), 5.70 (br s, 1H), 4.22 (m, 2H), 3.65 (m, 2H), 2.70 (s, 3H), 2.45 (br m, 2H), 1.50 (s, 9H).

### Description 103

### 4-(7-Fluoro-2-Methylquinolin-5-yl)piperidine-1-carboxylic acid tert-butyl ester (D103)

A solution of 4-(7-fluoro-2-methylquinolin-5-yl)-3,6-dihydro-2H-pyridine-1-carboxylic acid tert-butyl ester (D102) (0.6 g, 1.7 mmol) in ethanol (30 ml) was hydrogenated over 10% Pd—C (200 mg) at atmospheric pressure for 40 h at r.t. The reaction mixture was filtered through a celite pad which was then washed with ethanol (2×50 ml). The combined filtrates were then evaporated to give the title compound (D103) as a clear colourless oil (0.44 mg, 70%).

MS; (ES) m/z: 345.3 [MH⁺]. C₂₀H₂₆FN₂O₂ requires 344.

¹H-NMR (300 MHz, CDCl₃) δ: 8.25 (d, 1H), 7.6 (d, 1H), 7.20 (m, 1H), 7.1 (d, 1H), 4.30 (br m, 2H), 3.5 (t, 1H), 2.85 (br m, 2H), 2.7 (s, 3H), 1.85 (br m, 2H), 1.65 (br m, 2H), 1.40 (s, 9H), 1.38 (m, 1H).

### Description 104

### 7-Fluoro-2-methylquinolin-5-(1,2,3,6-tetrahydropyridin-4-yl]quinoline (D104)

A solution of 4-(7-fluoro-2-methylquinolin-5-yl)-3,6-dihydro-2H-pyridine-1-carboxylic acid tert-butyl ester (D103) (150 mg, 0.46 mmol) in DCM (10 ml) was treated with trifluoroacetic acid (0.35 ml, 0.46 mmol), stirred for 30 minutes and then stirred at r.t. for 16 h. The reaction mixture was made basic with saturated aqueous NaHCO₃ (10 ml), then the combined organics were dried (Na₂SO₄) and evaporated to give the title compound (D104) as a white solid (85 mg, 83%).

MS; (ES) m/z: 225 [MH⁺]. C₁₅H₁₆N₂ requires 224.

¹H-NMR (300 MHz, CDCl₃) δ: 8.25 (d, 1H), 7.87 (d, 1H), 7.58 (t, 1H), 7.23 (m, 2H), 5.75 (br m, 1H), 3.57 (m, 2H), 3.25 (m, 2H), 2.70 (s, 3H), 2.40 (m, 2H). NH not observed.

### Description 105

### 8-Nitro-2,3-dihydro-1,4-benzodioxin-6-carbonitrile (D105)

The title compound (D105) (0.2 g) was prepared according to Description 81 starting from 3,4-dihydroxy-5-nitrobenzonitrile (0.2 g, 1.11 mmol, prepared as described in J. Med. Chem 1989, 32(4), 841-846) in 87% yield.

¹H-NMR (CDCl₃, 343 K) δ: 7.79 (d, 1H); 7.34 (d, 1H); 4.50 (m, 2H); 4.40 (m, 2H).

### Description 106

### 8-Amino-2,3-dihydro-1,4-benzodioxin-6-carbonitrile (D106)

The title compound (D106) (0.2 g) was prepared according to Description 79 (Method B) starting from 8-nitro-2,3-dihydro-1,4-benzodioxin-6-carbonitrile (D105) (0.2 g, 0.97 mmol) in 58% yield

¹H-NMR (CDCl₃, 343 K) δ: 6.61 (d, 1H); 6.55 (d, 1H); 4.33 (m, 2H); 4.25 (m, 2H).

### Description 107

### 1,1-Dimethylethyl 4-(7-cyano-2,3-dihydro-1,4-benzodioxin-5-yl)-1-piperazinecarboxylate (D107)

The title compound (D107) (0.1 g) was prepared according to Description 82 starting from 8-amino-2,3-dihydro-1,4-benzodioxin-6-carbonitrile (D106) (0.1 g, 0.57 mmol), in 51% yield.

ESI POS; AQA: spray 3.5 KV/source 30 V/probe 250° C.: 346.31 (MH+)

### Description 108

### 8-(1-piperazinyl)-2,3-dihydro-1,4-benzodioxin-6-carbonitrile (D108)

The title compound (D108) was prepared according to Description 83 starting from 1,1-dimethylethyl 4-(7-cyano-2,3-dihydro-1,4-benzodioxin-5-yl)-1-piperazine-carboxylate (D107) in 100% yield.

¹H-NMR (CDCl₃, 343 K) δ: 9.99 (m br, 2H); 6.90 (d, 1H); 6.81 (d, 1H); 4.37-4.19 (m, 4H); 3.54-3.24 (m, 8H)

EI; TSQ 700; source 180° C.; 70 V; 200 uA: (M+.) 245, 203, 132, 104.

### EXAMPLES

### General procedure for the alkylation of arylpiperazines with 6-(2-chloroethyl)-4H-benzo[1,4]oxazin-3-one (D4)

To a suspension of the appropriate arylpiperazine in MIBK or NMP as solvent, 6-(2-chloroethyl)-4H-benzo[1,4]oxazin-3-one (D4) (1.3 eq), NaI (1.3 eq), Na₂CO₃ (1.3 eq) were added. The reaction mixture was heated at 120° C. for 12 h. and the solvent removed by SCX cartridge. The crude material was purified on SPE cartridge (Si) eluting with a gradient from 100% DCM to 80% DCM 20% MeOH to afford the final compounds (yields ranged from 16 to 85%). The free bases were generally converted into the hydrocloride salt by dissolving in MeOH or diethylether and adding 1M solution of hydrochloric acid (3 eq.) in dry MeOH. The final salts were then recovered by filtration.

### Example 1

### 6-{2-[4-(2-Methylquinolin-5-yl)piperazin-1-yl]ethyl}-4H-benzo[1.4]oxazin-3-one hydrochloride salt (E1)

The title compound (E1) was prepared in 65% yield according to the general alkylation procedure starting from 2-methyl-5-piperazin-1-ylquinoline (D3).

MS; (ES) m/z: 403.2 [MH]⁺. C₂₄H₂₆N₄O₂.HCl requires 402.

¹H-NMR (500 MHz, DMSO) δ: 11.05 (bs, 1H), 10.81 (s, 1H), 8.88 (bs, 1H), 7.92 (bs, 2H), 7.79 (bs, 1H), 7.42 (s, 1H), 6.95 (d, 1H), 6.86 (dd, 1H), 6.80 (d, 1H), 4.55 (s, 2H), 3.70 (d, 2H), 3.6-3.5 (m, 4H), 3.35 (m, 2H), 3.35 (m, 2H), 3.05 (m, 2H), 2.88 (bs, 3H).

### Example 2

### 6-{2-[4-(2,7-Dimethylquinolin-5-yl)piperazin-1-yl]ethyl}-4H-benzo[1,4]oxazin-3-one hydrochloride salt (E2)

The title compound (E2) was prepared from (2,7-dimethylquinolin-5-yl)piperazine (D8) according to the general procedure described above in 30% yield.

¹H-NMR (300 MHz, CDCl₃) δ: 8.50 (br s, 1H), 8.25 (d, 1H), 7.50 (s, 1H), 7.15 (d, 1H), 6.80-6.90 (m, 3H), 6.65 (s, 1H), 4.55 (s, 2H), 3.10 (br s, 4H), 2.75 (br s, 4H), 2.68 (m, 2H), 2.65 (s, 3H), 2.47 (s, 3H), 1.80 (br m, 2H).

### Example 3

### 6-{2-[4-(7-Chloro-2-methylquinolin-5-yl)piperazin-1-yl]ethyl}-4H-benzo[1,4]oxazin-3-one hydrochloride salt (E3)

The title compound (E3) was prepared in 40% yield according to the general procedure from 7-chloro-2-methyl-5-piperazin-1-ylquinoline (D9).

MS; (ES) m/z: 437.1 [MH]⁺. C₂₄H₂₅ClN₄O₂.HCl requires 436.

¹H-NMR (500 MHz, DMSO) δ: 10.90 (s, 1H), 11.10 (s, 1H), 8.50 (d, 1H), 7.80 (s, 1H), 7.60 (d, 1H), 7.40 (s, 1H), 7.00 (d, 1H), 6.80 (d, 1H), 4.50 (s, 2H), 3.80-3.00 (m, 4H), 3.10-2.80 (m, 8H), 2.70 (s, 3H).

### Example 4

### 6-[2-(4-Quinolin-4-ylpiperazin-1-yl)ethyl]-4H-benzo[1.4]oxazin-3-one hydrochloride salt (E4)

The title compound (E4) was prepared from 4-piperazin-1-ylquinoline as above in 16% yield.

¹H-NMR (300 MHz, CDCl₃) δ: 9.02 (brs, 1H), 8.70 (d, 1H), 8.00 (m, 2H), 7.60 (t, 1H), 7.45 (t, 1H), 6.80-6.90 (m, 3H), 6.67 (s, 1H), 4.55 (s, 2H), 3.25 (br s, 4H), 2.75 (br s, 4H), 2.65 (m, 2H), 1.85 (br m, 2H).

### Example 5

### 6-{2-[4-(2-Methylquinazolin-5-yl)piperazin-1-yl]ethyl}-4H-benzo[1.4]oxazin-3-one hydrochloride salt (E5)

The title compound (E5) was prepared in 85% yield according to the general procedure starting from 2-methyl-5-piperazin-1-ylquinazoline (D12).

MS; (ES) m/z: 403 [MH⁺].

¹H-NMR (500 MHz, DMSO) δ: 11.4 (s, 1H), 10.81 (s, 1H), 9.58 (s, 1H), 7.89 (t, 1H), 7.61 (d, 1H); 7.27 (d, 1H); 6.94 (d, 1H); 6.85 (dd, 1H); 6.81 (d, 1H); 4.54 (s, 2H); 3.68 (d, 2H); 3.56 (d, 2H); 3.47 (m, 2H); 3.36 (m, 2H); 3.33 (m, 2H); 3.07 (m, 2H); 2.77 (s, 3H).

### Example 6

### 6-{2-[4-(2,3-Dihydrobenzo[1,4]dioxin-5-yl)piperazin-1-yl]ethyl}-4H-benzo[1.4]oxazin-3-one hydrochloride salt (E6)

The title compound was prepared in 30% yield according to the general procedure starting from 1-(2,3-dihydrobenzo[1,4]dioxin-5-yl)piperazine.

MS; (ES) m/z: 396 [MH⁺].

¹H-NMR (500 MHz, DMSO) δ: 10.63 (s, 1H), 6.84 (m, 2H); 6.7 (m, 2H); 6.58 (m, 2H); 6.46 (m 2H); 4.51 (s, 2H); 4.20 (m, 4H); 2.95 (m, 4H); 2.54 (m, 4H); 2.63-2.54 (m, 4H).

### Example 7

### 6-{2-[4-(6-Methoxyquinolin-8-yl)piperazin-1-yl]ethyl}-4H-benzo[1.4]oxazin-3-one hydrochloride salt (E7)

The title compound (E7) was prepared in 19% yield according to the general procedure starting from 6-methoxy-8-piperazin-1-ylquinoline.

MS; (ES) m/z: 419 [MH⁺].

¹H-NMR (500 MHz, DMSO) δ: 10.63 (s, 1H), 8.65 (dd, 1H); 8.16 (dd, 1H), 7.42 (m, 1H); 6.89 (d, 1H); 6.85 (d, 1H); 6.7 (m, 2H); 6.79 (dd, 1H); 6.77 (d, 1H); 6.67 (d, 1H);

### Example 8

### 6-[2-(4-Quinolin-8-yl)piperazin-1-yl)ethyl]-4H-benzo[1.4]oxazin-3-one hydrochloride salt (E8)

The title compound (E8) was prepared in 20% yield according to the general procedure starting from 8-piperazin-1-ylquinoline (E8).

MS; (ES) m/z: 389.2 [MH⁺].

¹H-NMR (500 MHz, DMSO) δ: 10.64 (s, 1H), 8.84 (dd, 1H); 8.26 (dd, 1H), 7.50 (m, 1H); 7.48 (d, 1H); 7.47 (d, 1H); 7.14 (dd, 1H); 6.86-6.7 (m, 3H); 4.5 (s, 2H); 3.36 (m, 4H); 2.70 (m, 6H); 2.55 (m, 2H).

### Example 9

### 6-{2-[4-(1H-Indol-4-yl)piperazin-1-yl]ethyl}-4H-benzo[1.4]oxazin-3-one hydrochloride salt (E9)

The title compound was prepared in 22% yield according to the general procedure from 4-piperazin-1-yl-1H-indole.

MS; (ES) m/z: 377.3 [MH⁺].

¹H-NMR (500 MHz, DMSO) δ: 11.00 (s, 1H), 10.64 (s, 1H); 7.22 (t, 1H); 6.99 (d, 1H); 6.94 (t, 1H); 6.85 (d, 1H); 6.79 (dd, 1H); 6.77 (d, 1H); 6.43 (d, 1H); 6.35 (t, 1H); 4.51 (s, 2H); 3.15 (m, 4H); 2.67 (m, 6H); 2.56 (m, 2H).

### Example 10

### 6-{2-[4-(7-Chloro-2-methylquinolin-5-yl)piperazin-1-yl]ethyl}-7-fluoro-4H-benzo[1,4]oxazin-3-one (E10)

The title compound (E10) was prepared in 32% yield according to the general procedure starting from 7-chloro-2-methyl-5-piperazin-1-ylquinoline (D9) and 6-(2-chloroethyl)-7-fluoro-4H-benzo[1,4]oxazin-3-one (D24).

MS; (ES) m/z: 456.2 [MH]⁺.

¹H-NMR (500 MHz, DMSO) δ: 11.40 (s, 1H); 10.86 (s, 1H); 8.73 (s, 1H): 7.96 (d, 1H); 7.72 (d, 1H); 7.04 (d, 1H); 6.95 (d, 1H); 6.83 (d, 1H); 4.58 (s, 2H); 3.71-3.52 (m, 4H); 3.52-3.38 (m, 6H); 3.32 (m, 2H); 3.09 (m, 2H); 2.84 (s, 3H).

### Example 11

### 4-Methyl-6-{2-[4-(2-methylquinolin-5-yl)piperazin-1-yl]ethyl}-4H-benzo[1.4]oxazin-3-one hydrochloride salt (E11)

A solution of 6-{2-[4-(2-methylquinolin-5-yl)piperazin-1-yl]ethyl}-4H-benzo[1.4]-oxazin-3-one hydrochloride salt (E1) (56 mg, 0.13 mmol) in dry DMF (4 mL) was cooled to 0° C. and 60% sodium hydride (21 mg; 4.0 eq) was added. The reaction mixture was stirred under nitrogen at 0° C. for 40 minutes and then methyl iodide (9 μL) was added. The mixture was allowed to stir at 0° C. for 1 h, poured into a saturated aq. solution of ammonium chloride (4 mL) and extracted into ethyl acetate (3×3 mL). The combined organic layers were dried (Na₂SO₄) and concentrated in vacuo. The residue was passed through a SCX cartridge and the resulting crude product was dissolved in DCM (2 mL) and treated with 2.0 M HCl in diethyl ether (1.1 eq). The mixture was stirred at 0° C. for 30 min, then concentrated in vacuo to give the title compound (E11) as a yellow solid (32 mg; yield 55%).

MS; (ES) m/z 417.3 [MH⁺]. C₂₅H₂₈N₄O₂.HCl requires 416.

¹H-NMR (500 MHz, DMSO) δ: 8.36 (d, 1H), 7.69 (d, 1H), 7.56 (t, 1H), 7.22 (d, 1H), 7.05 (d, 1H), 6.86 (m, 3H), 4.57 (s, 2H), 3.34 (s, 3H), 3.12 (m, 4H), 2.85-2.75 (m, 6H), 2.70 (m, 2H), 2.70 (s, 3H).

### Example 12

### 6-{2-[4-(2-Methylquinolin-5-yl)piperazin-1-yl]ethanoyl}-4H-benzo[1.4]oxazin-3-one hydrochloride salt (E12)

2-Methyl-5-piperazin-1-yl-quinoline (D3) (50 mg, 0.22 mmol, 1.0 eq.) and 6-(2-chloroethanoyl)-4H-benzo[1,4]oxazin-3-one (65 mg, 0.29 mmol, 1.3 eq.) were added to a solution of N,N-diisopropylethylamine (1.0 mL) in dry acetonitrile (3 mL). The reaction mixture was stirred at reflux for 7 h, then allowed to cool to r.t. and concentrated in vacuo. The residue was dissolved in water (15 mL) and ethyl acetate (15 mL) and shaken. The organic phase was separated, dried (Na₂SO₄) and concentrated in vacuo. The crude product was passed through a SCX cartridge then purified by flash chromatography, eluting with 2% methanol in DCM. The resulting product was dissolved in methanol (3 mL) and treated with 1.25 M HCl in ethanol (1 mL). The mixture was stirred at r.t. for 0.5 h, then concentrated in vacuo to give the title compound (E12) as a yellow solid (56 mg, yield 56%).

MS; (ES) m/z: 417 [MH⁺]. C₂₄H₂₄N₄O₃.HCl requires 416.

¹H-NMR (500 MHz, DMSO) δ: 11.03 (s, 1H), 10.6 (bs, 1H), 8.88 (bs, 1H), 7.94 (bs, 2H), 7.79 (bs, 1H), 7.67 (d, 1H), 7.54 (dd, 1H), 7.52 (s, 1H), 7.18 (d, 1H), 5.15 (bs, 2H), 4.75 (s, 2H), 3.8-3.5 (m, 4H), 2.88 (bs, 3H).

### Example 13

### 6-{1-Hydroxy-2-[4-(2-methylquinolin-5-yl)piperazin-1-yl]ethyl}-4H-benzo[1.4]oxazin-3-one hydrochloride salt (E13)

A stirred suspension of 6-{2-[4-(2-methylquinolin-5-yl)piperazin-1-yl]ethanoyl}-4H-benzo[1.4]oxazin-3-one (E12) (47 mg, 0.10 mmol, 1.0 eq.) in dry ethanol (2 mL) was cooled to 0° and sodium borohydride (15 mg, 0.40 mmol, 4.0 eq) was added portionwise. The reaction mixture was stirred at 0° under nitrogen for 3 h. The reaction was quenched at r.t. with a saturated aq. solution of ammonium chloride (10 mL) and extracted into ethyl acetate (3×15 mL). The organic layers were combined, dried (Na₂SO₄) and concentrated in vacuo. The crude product was purified by flash chromatography, eluting with 2% methanol in DCM. The resulting product was dissolved in methanol (3 mL) and treated with 1.25 M HCl in ethanol (1 mL). The mixture was stirred at r.t. for 0.5 h, then concentrated in vacuo to give the title compound (E13) as a yellow solid (15 mg, yield 33%).

MS; (ES) m/z: 419 [MH⁺]. C₂₄H₂₆N₄O₃.HCl requires 418.

¹H-NMR (500 MHz, DMSO) δ: 10.62 (s, 1H), 10.27 (bs, 1H), 8.63 (d, 1H), 7.80 (m, 2H), 7.58 (d, 1H), 7.32 (d, 1H), 7.04-6.90 (m, 3H), 5.15 (d, 1H), 4.55 (s, 2H), 3.75 (bm, 4H), 3.35 (bm, 5H), 3.30 (m, 2H), 2.78 (s, 3H).

### Example 14

### 6-{2-[4-(2-Methyl-4-(2-methylquinolin-5-yl)piperazin-1-yl]ethyl}-4H-benzo[1.4]oxazin-3-one hydrochloride salt (E14)

The title compound (E14) was prepared in 36% yield according to the general procedure starting from 2-Methyl-5-(3-methylpiperazin-1-yl)quinoline (D13).

MS; (ES) m/z: 417.2 [MH⁺].

¹H-NMR (500 MHz, DMSO) δ: 10.63 (s, 1H); 8.36 (d, 1H); 7.57 (m, 2H); 7.38 (d, 1H); 7.07 (dd, 1H); 6.84 (d, 1H); 6.80 (dd, 1H); 6.77 (d, 1H); 4.52 (s, 2H); 2.62 (s, 3H); 3.2 (m, 4H); 2.5 (m, 4H).

### Example 15

### 6-{2-[3-Methyl-4-(2-methylquinolin-5-yl)piperazin-1-yl]ethyl}-4H-benzo[1.4]oxazin-3-one hydrochloride salt (E15)

The title compound (E15) was prepared in 58% yield following the general procedure from 2-methyl-5-(2-methylpiperazin-1-yl)quinoline (D14).

MS; (ES) m/z: 417.3 [MH⁺]. C₂₅H₂₈N₄O₂ requires 416.52.

¹H NMR (500 MHz, DMSO) δ: 12.27 (bs, 1H), 10.77 (s, 1H), 9.07 (bs, 1H), 8.09 (d, 1H), 7.98 (t, 1H), 7.82 (d, 1H), 7.64 (d, 1H), 6.91 (d, 1H), 6.83 (dd, 1H), 6.77 (d, 1H), 4.51 (s, 2H), 3.75 (m, 1H), 3.6-3.2 (m, 6H), 3.5 (m, 2H), 3.05 (m, 2H), 2.87 (s, 3H), 0.79 (d, 3H).

### Example 16

### 6-{2-[2-Methyl-4-(2-methylquinolin-5-yl)piperazin-1-yl]ethyl}-4H-benzo[1.4]oxazin-3-one hydrochloride salt (E16)

The title compound (E16) was prepared in 28% yield according to the general procedure starting from 2-methyl-5-(3-methylpiperazin-1-yl)quinazoline (D15).

MS; (ES) m/z: 418.4 [MH⁺]. C₂₄H₂₇N₅O₂ requires 417.52.

¹H NMR (500 MHz, DMSO) δ: 10.74 (sa, 1H), 9.59 (s, 1H), 7.85 (t, 1H), 7.58 (d, 1H), 7.24 (d, 1H), 6.91-6.75 (m, 3H), 4.51 (s, 2H), 3.95 (m, 1H), 3.8-3.2 (m, 4H), 3.5 (m, 2H), 3.05 (m, 2H), 3 (m, 2H), 2.73 (s, 3H), 1.39 (d, 3H).

### Example 17

### 6-{2-[4-(2-Methylquinolin-5-yl)-3,6-dihydro-2H-pyridin-1-yl]ethyl}-4H-benzo[1,4]oxazin-3-one (E17)

The title compound (E17) was prepared from 2-methyl-5-(1,2,3,6-tetrahydropyridin-4-yl]quinoline (D17) and 6-(2-chloro)ethyl-4H-benzo[1,4]oxazin-3-one (D4) according to the general procedure described above in 20% yield.

MS; (ES) m/z: 400 [MH⁺]. C₂₅H₂₅N₃O₂ requires 399.

¹H-NMR (300 MHz, CDCl₃) δ: 8.30 (br s, 1H), 8.25 (d, 1H), 7.88 (d, 1H), 7.57 (t, 1H), 7.15-7.25 (m, 2H), 6.85 (m, 2H), 6.65 (s, 1H), 5.72 (m, 1H), 4.55 (s, 2H), 3.25 (m, 2H), 2.70-2.90 (m, 6H), 2.70 (s, 3H), 2.55 (m, 2H).

### Example 18

### 6-{2-[4-(2-Methylquinolin-5-yl)piperidin-1-yl]ethyl}-4-H-benzo[1,4]oxazin-3-one hydrochloride salt (E18)

The title compound (E18) was prepared from 2-methyl-5-piperidin-4-ylquinoline (D19) and 6-(2-chloro)ethyl-4H-benzo[1,4]oxazin-3-one (D4) according to the general procedure described above in 31% yield.

MS; (ES) m/z: 402 [MH⁺]. C₂₅H₂₇N₃O₂ requires 401.

¹H-NMR (300 MHz, CDCl₃) δ: 9.25 (br s, 1H), 8.25 (d, 1H), 7.85 (d, 1H), 7.57 (t, 1H), 7.35 (d, 1H), 7.25 (t, 1H), 6.82 (m, 2H), 6.65 (s, 1H), 4.55 (s, 2H), 3.05-3.30 (m, 3H), 2.60-2.80 (m, 4H), 2.70 (s, 3H), 2.20-2.40 (m, 2H), 1.90-2.15 (m, 4H).

### Example 19

### 6-{2-[4-(2-Methylquinolin-5-yl)-[1,4]diazepan-1-yl]ethyl}-4H-benzo[1,4]oxazin-3-one hydrochloride salt (E19)

The title compound (E19) was prepared by the general procedure reported above in 60% yield starting from 5-[1,4]diazepan-1-yl-2-methylquinoline (D21).

MS; (ES) m/z: 417.3 [MH⁺]. C₂₅H₂₈N₄O₂ requires 416.52.

¹H NMR (500 MHz, DMSO) δ: 12.27 (bs, 1H), 10.77 (s, 1H), 9.07 (bs, 1H), 8.09 (d, 1H), 7.98 (t, 1H), 7.82 (d, 1H), 7.64 (d, 1H), 6.91 (d, 1H), 6.83 (dd, 1H), 6.77 (d, 1H), 4.51 (s, 2H), 3.75 (m, 1H), 3.6-3.2 (m, 6H), 3.5 (m, 2H), 3.05 (m, 2H), 2.87 (s, 3H), 0.79 (d, 3H).

### Example 20

### 6-{2-[4-(2-Methylquinazolin-5-yl)-[1,4]diazepan-1-yl]ethyl}-4H-benzo[1,4]oxazin-3-one hydrochloride salt (E20)

The title compound (E20) was prepared in 32% yield by the general procedure reported above from 5-[1,4]-diazepan-1-yl-2-methylquinazoline (D22).

MS; (ES) m/z: 418.4 [MH⁺]. C₂₄H₂₇N₅O₂ requires 417.52.

¹H NMR (500 MHz, DMSO) δ: 10.85 (sa, 1H), 10.78 (s, 1H), 9.63 (s, 1H), 7.87 (t, 1H), 7.52 (d, 1H), 7.24 (d, 1H), 6.92 (d, 1H), 6.85 (dd, 1H), 6.78 (d, 1H), 4.53 (s, 2H), 3.8-3.5 (m, 6H), 3.5-3.47 (m, 4H), 3.03 (m, 2H), 2.85 (s, 3H), 2.4-2.15 (m, 2H).

### Example 21

### 7-Fluoro-6-{2-[4-(2-methylquinolin-5-yl)piperazin-1-yl]ethyl}-4H-benzo[1.4]oxazin-3-one hydrochloride salt (E21)

The title compound (E21) was prepared according to the general alkylation procedure from 2-methyl-5-piperazin-1-ylquinoline (D3) and 6-(2-chloroethyl)-7-fluoro-4H-benzo[1,4]oxazin-3-one (D24).

MS; (ES) m/z: 421.2 [MH]⁺. C₂₄H₂₅FN₄O₂.HCl requires 420.

¹H-NMR (500 MHz, DMSO) δ: 10.90 (m, 2H), 8.40 (bs, 1H), 7.60 (m, 2H), 7.40 (d, 1H), 7.40 (d, 1H), 7.10 (m, 1H), 7.00 (d, 1H), 4.60 (s, 2H), 3.80-3.00 (m, 4H), 3.10-2.80 (m, 8H), 2.70 (s, 3H).

### Example 22

### 6-{3-[4-(2-Methylquinolin-5-yl)-piperazin-1-yl]-propyl}-4H-benzo[1,4]-oxa-zin-3-one acetic acid salt (E22)

A mixture of (4-{3-[4-(2-methylquinolin-5-yl)piperazin-1-yl]propyl}-2-nitro-phenoxy)-acetic acid methyl ester (D29) (0.19 g, 0.4 mmol) and iron (0.9 g) in glacial acetic acid (5 mL) was stirred vigorously at r.t. for 4 h under argon. The reaction mixture was diluted with ethyl acetate (100 mL) and filtered through a Celite pad and washed with ethyl acetate. The fitrate and washings were combined, the solvent was evaporated and the residue was co-evaporated with toluene (2×30 mL). The product was purified by column chromatography on silica gel (eluting with a methanol-DCM gradient) to give the title compound (E22) as an acetic acid salt (0.13 g, 71%); ¹H-NMR (500 MHz, CDCl₃), δ: 1.88 (2H, q, J=7.6 Hz), 2.52 (2H, br t, J=7.6 Hz), 2.62 (2H, t, J=7.6 Hz), 2.73 (3H, s), 2.77 (4H, br m), 3.14 (4H, br t, J=4.4 Hz), 4.60 (2H, s), 6.65 (1H, d, J=1.2 Hz), 6.82 (1H, dd, J=8.2 Hz, 1.6 Hz), 6.9 (1H, d, J=8.2 Hz), 7.08 (1H, d, J=7.6 Hz), 7.25 (1H, d, J=8.5 Hz), 7.58 (1H, t, J=7.9 Hz), 7.73 (1H, d, J=8.5 Hz), 8.10 (1H, br s), 8.37 (1H, d, J=8.5 Hz); MS: m/z (MH⁺)=417/418.

### Example 23

### 6-{3-[4-(7-Fluoro-2-methylquinolin-5-yl)piperazin-1-yl]-propyl}-4H-benzo-[1,4]oxazin-3-one (E23)

The title compound (E23) was prepared as described in Example 22 from 7-fluoro-2-methyl-5-piperazin-1-ylquinoline (D30).

MS: m/z (MH⁺)=435/436.

¹H-NMR (400 MHz, CDCl₃) δ_{H} 1.80 (2H, br q, J=7.0 Hz), 2.44 (2H, br t, J=7.4 Hz), 2.55 (2H, t, J=7.4 Hz), 2.64 (3H, s), 2.69 (4H, br s), 3.06 (4H, br s), 4.53 (2H, s), 6.59 (1H, t, J=8.0 Hz), 6.76 (1H, d, J=8.0 Hz,), 6.83 (1H, d, J=8.0 Hz,), 7.12 (1H, d, J=8.8 Hz), 7.27 (1H, dd, J=10.0 Hz, 2.4 Hz), 8.20 (1H, d, J=8.8 Hz), 8.37 (1H, br s).

### Example 24

### 6-{3-[4-(2-Methylquinolin-5-yl)-piperazin-1-yl]-propanoyl}-4H-benzo[1,4]-oxa-zin-3-one hydrochloric acid salt (E24)

2-Methyl-5-piperazin-1-yl-quinoline (D3) (0.50 g, 2.20 mmol) and 6-(3-chloropropanonyl)-4H-benzo[1,4]oxazin-3-one (0.65 g, 2.86 mmol, 1.3 eq.) (C. R. Acad. Sci., Ser. C 1970, 270(19), 1601-4) were added to a solution of N,N-diisopropylethylamine (10 mL) in dry acetonitrile (30 mL). The reaction mixture was stirred at reflux for 7 h, then allowed to cool to r.t. and concentrated in vacuo. The residue was dissolved in water (50 mL) and ethyl acetate (50 mL) and shaken. The organic phase was separated, dried (Na₂SO₄) and concentrated in vacuo. The crude product was passed through a SCX cartridge, then purified by flash chromatography, eluting with 3% methanol in DCM, to afford the title compound (E24) as a yellowish solid (0.81 g, 85%).

MS; (ES) m/z: 431 [MH⁺]. C₂₅H₂₆N₄O₃ requires 430

¹H-NMR (500 MHz, DMSO) δ: 10.83 (bs, 1H), 8.34 (d, 1H), 7.67 (d, 1H), 7.58 (m, 2H), 7.51 (d, 1H), 7.37 (d, 1H), 7.09 (dd, 1H), 7.05 (d, 1H), 4.68 (s, 2H), 3.16 (t, 2H), 3.00 (bs, 4H), 2.70 (bs, 4H), 2.68 (t, 2H), 2.62 (s, 3H).

### Example 25

### 6-{1-Hydroxy-3-[4-(2-methylquinolin-5-yl)-piperazin-1-yl]-propyl}-4H-benzo-[1,4]oxazin-3-one hydrochloric acid salt (E25)

A stirred suspension of 6-{3-[4-(2-methylquinolin-5-yl)-piperazin-1-yl]-propanoyl}-4H-benzo[1,4]-oxa-zin-3-one hydrochloric acid salt (E24) (300 mg, 0.70 mmol) in dry methanol (10 mL) was cooled to 0° and sodium borohydride (106 mg, 2.8 mmol, 4.0 eq) was added portionwise. The reaction mixture was stirred at 0° under nitrogen for 4 h then quenched at r.t. with a saturated aq. solution of ammonium chloride (50 mL) and extracted into ethyl acetate (3×50 mL). The organic layers were combined, dried (Na₂SO₄) and concentrated in vacuo. The residue was purified by flash chromatography, eluting with 5% methanol in DCM, to give the title compound (E25) as a yellowish solid (266 mg, 88%).

MS; (ES) m/z: 433 [MH⁺]. C₂₅H₂₈N₄O₃ requires 432.

¹H-NMR (300 MHz, DMSO) δ: 10.65 (s, 1H), 8.30 (d, 1H), 7.55 (m, 2H), 7.32 (d, 1H), 7.10 (m, 1H), 6.90 (s, 1H), 6.80 (s, 2H), 5.40 (bs, 1H), 4.60 (m, 1H), 4.50 (s, 2H), 3.30 (bm, 4H), 2.65 (bm, 4H), 2.60 (s, 3H), 1.75 (m, 2H).

### Example 26

### 6-{(E)-3-[4-(2-Methylquinolin-5-yl)piperazin-1-yl]propenyl}-4H-benzo[1,4]-oxa-zin-3-one hydrochloric acid salt (E26)

A stirred suspension of 6-{1-hydroxy-3-[4-(2-methylquinolin-5-yl)-piperazin-1-yl]-propyl}-4H-benzo-[1,4]oxazin-3-one hydrochloric acid salt (E25) (50 mg, 0.116 mmol) and p-toluenesulfonic acid (110 mg, 0.58 mmol, 5.0 eq) in dry toluene (3 mL) was refluxed for 4 h. The reaction was quenched at r.t. with a saturated solution of sodium hydrogencarbonate (10 mL) and extracted into ethyl acetate (3×15 mL). The organic layers were combined, dried (Na₂SO₄) and concentrated in vacuo. The residue was purified by flash chromatography, eluting with 2% methanol in DCM, to give the title compound (E26) as a yellow solid (20 mg, 42%).

MS; (ES) m/z: 415 [MH⁺]. C₂₅H₂₆N₄O₂ requires 414.

¹H-NMR (500 MHz, DMSO) δ: 10.69 (bs, 1H), 8.38 (d, 1H), 7.57 (m, 2H), 7.36 (d, 1H), 7.10 (d, 1H), 7.01 (dd, 1H), 6.94 (d, 1H), 6.88 (d, 1H), 6.51 (d, 1H), 6.15 (m, 1H), 4.55 (s, 2H), 3.19 (d, 2H), 3.03 (bm, 4H), 2.70 (bm, 4H), 2.62 (s, 3H).

### Example 27

### 6-{4-[4-(2-Methylquinolin-5-yl)piperazin-1-yl]butyl}-4H-benzo[1,4]oxazin-3-one hydrochloric acid salt (E27)

A mixture of 6-(4-chlorobutyl)-4H-benzo[1,4]oxazin-3-one (30 mg), 2-methylquinolylpiperazine (40 mg), sodium iodide (35 mg), and sodium carbonate (50 mg) were suspended in methyl isobutyl ketone (4 mL). The reaction mixture was heated at 120° C. for 6 h., cooled to r.t., diluted with ethyl acetate (10 mL), filtered and concentrated to dryness. The oily residue was purified by flash chromatography eluting with a 97/3 mixture of DCM/methanol to afford the title compound as a free base which was converted to the corresponding hydrochloride salt (E27) (yield 40%) using a 1M solution of HCl in diethyl ether.

¹H-NMR (500 MHz, DMSO) δ: 10.55, (bs, 1H), 10.49 (s, 1H), 8.67 (d, 1H), 7.81 (m, 2H), 7.62 (d, 1H), 7.31 (d, 1H), 6.85 (d, 1H), 6.77 (m, 2H), 4.50 (s, 2H), 3.60 (m, 2H), 3.40 (m, 2H), 3.50-3.10 (m, 4H), 3.18 (m, 2H), 2.79 (s, 3H), 2.57 (t, 2H), 1.79 (m, 2H), 1.63 (m, 2H).

### Example 28

### 6-{4-[4-(2-Methylquinolin-5-yl)piperazin-1-yl]-cyclohex-1-enyl}-4H-benzo[1,4]-oxazin-3-one hydrochloric acid salt (E28)

Glacial acetic acid (0.3 mL) was added to a stirred mixture of 6-(4-oxocyclohex-1-enyl)-4H-benzo[1,4]oxazin-3-one (D32) (58 mg, 0.24 mmol), 2-methyl-5-piperazin-1-yl-quinoline (D3) (82 mg, 0.36 mmol, 1.5 eq.) and sodium triacetoxyborohydride (76 mg, 0.36 mmol, 1.5 eq) in 1,2-dichloroethane (4 mL) at r.t. The reaction mixture was stirred at r.t. overnight, then quenched with a saturated aq. solution of sodium hydrogencarbonate (20 mL) and extracted with ethyl acetate (3×20 mL). The organic layers were combined, dried (Na₂SO₄) and concentrated in vacuo. The crude product was passed through a SCX cartridge, then purified by flash chromatography, eluting with 3% methanol in dichloromethane, to afford the title compound (E28) as a yellowish solid (7 mg, yield 6%).

MS; (ES) m/z: 455 [MH⁺]. C₂₈H₃₀N₄O₂ requires 454.

¹H-NMR (500 MHz, DMSO) δ: 10.65 (s, 1H), 8.36 (d, 1H), 7.58 (m, 2H), 7.11 (dd, 1H), 7.08 (d, 1H), 6.99 (dd, 1H), 6.92 (d, 1H), 6.89 (d, 1H), 6.0 (bs, 1H), 4.54 (s, 2H), 3.03 (bs, 4H), 2.84 (bs, 2H), 2.79 (bs, 2H), 2.62 (s, 3H), 2.62 (m, 1H), 2.48 (m, 1H), 2.40 (m, 2H), 2.2 (m, 2H), 1.55 (m, 1H).

### Example 29

### 6-{4-[4-(2-Methylquinazolin-5-yl)piperazin-1-yl]butyl}-4H-benzo[1,4]oxazin-3-one hydrochloric acid salt (E29)

A mixture of 6-(4-chlorobutyl)-4H-benzo[1,4]oxazin-3-one (30 mg), 2-methyl-5-piperazin-1-ylquinazoline (D12) (40 mg), 5-(2-methylquinazolyl)piperazine (35 mg), sodium iodide (35 mg), and sodium carbonate (50 mg) were suspended in methyl isobutyl ketone (4 mL). The reaction mixture was heated at 120° C. for 16 h., cooled to r.t., concentrated under reduced pressure, diluted with DCM (50 ml) and washed with water (25 ml). The organic phase was separated and the solvent removed under reduced pressure to give an oily residue that was purified by flash cromatography eluting with DCM/methanol in a gradient system (100/0 to 50/50) to afford a crude mixture. The mixture was further purified by preparative HPLC using a reverse phase column [Waters X Terra C₁₈ eluting with water+0.1% TFA (solvent A)/ACN+0.1% TFA (solvent B), in gradient at 43 mL/min, flow] to give a solution containing the title compound. A 5% solution of sodium bicarbonate was added until a basic pH was obtained and the acetonitrile was removed under reduced pressure. The aqueous phase was extracted with DCM (3×15 mL). The combined organics were dried (Na₂SO₄) and evaporated under reduced pressure to afford the free base of the title compound which was converted into the corresponding hydrochloride salt (E29) (37 mg, 40%).

MS; (ES) m/z: 432 [MH]⁺.

¹H-NMR (500 MHz, DMSO) δ: 10.35, (s, 1H), 9.58 (s, 1H), 9.49 (bs, 1H), 7.87 (t, 1H), 7.61 (d, 1H), 7.62 (d, 1H), 7.25 (d, 1H), 6.88 (d, 1H), 6.77 (m, 1H), 6.72 (d, 1H), 4.52 (s, 2H), 3.23 (m, 2H), 3.60-3.10 (m, 8H), 2.76 (s, 3H), 2.54 (m, 2H), 1.69 (m, 2H), 1.58 (m, 2H).

### Example 30

### 6-{2-[4-(2-Methylquinolin-5-yl)piperazin-1-yl]ethoxy}-4-H-benzo[1,4]oxazin-3-one (E30)

The title compound (E30) was prepared from 2-methyl-5-piperazin-1-ylquinoline (D3) and 6-(2-bromoethoxy)-4H-benzo[1,4]oxazin-3-one (D36) according to the general procedure described for Example 1. Yield 68%.

MS; (ES) m/z: 419-[MH⁺]. C₂₄H₂₆N₄O₃ requires 418.

¹H-NMR (300 MHz, CDCl₃) δ: 9.50 (br s, 1H), 8.35 (d, 1H), 7.65 (d, 1H), 7.55 (t, 1H), 7.20 (d, 1H), 7.00 (d, 1H), 6.83 (d, 1H), 6.45 (m, 2H), 4.50 (s, 2H), 4.05 (t, 2H), 3.32 (t, 2H), 3.05 (m, 2H), 2.85 (m, 2H), 2.80 (s, 3H), 2.35 (m, 2H), 2.00 (m, 2H).

### Example 31

### 4-Methyl-6-{2-[4-(2-methylquinolin-5-yl)piperazin-1-yl]ethoxy}-4-H-benzo[1,4]oxazin-3-one (E31)

A solution of 6-{2-[4-(2-methylquinolin-5-yl)piperazin-1-yl]-ethoxy}-4-H-benzo-[1,4]-oxazin-3-one (E30) (20 mg, 0.05 mmol) in DMF at 0° C. under nitrogen was treated with sodium hydride (2 mg, 0.08 mmol) and stirred at 0° C. for 15 min. Methyl iodide (0.01 mL, 0.16 mmol) was added and the solution allowed to warm to room temp. over 3 h. The reaction mixture was then partitioned between water (20 mL) and DCM (3×25 mL). The combined organics were dried (Na₂SO₄) and chromatographed (eluent 5% MeOH/CH₂Cl₂) to afford the title compound (E31) as a yellow oil (2 mg, 10%).

MS; (ES) m/z: 433 [MH⁺]. C₂₅H₂₈N₄O₃ requires 432.

### Example 32

### 7-Fluoro-6-{2-[4-(7-fluoro-2-methylquinolin-5-yl)piperazin-1-yl]ethyl}-4H-benzo[1,4]oxazin-3-one hydrochloride salt (E32)

The title compound (E32) was prepared in 27% yield according to the general procedure starting from 7-fluoro-2-methyl-5-piperazin-1-ylquinoline (D30) and 6-(2-chloroethyl)-7-fluoro-4H-benzo[1,4]oxazin-3-one (D24).

MS; (ES) m/z: [MH]⁺. 439.2

¹H-NMR (500 MHz, DMSO) δ: 11.42 (s, 1H); 10.86 (s, 1H); 8.79 (d, 1H); 7.70 (d, 1H); 7.70 (d, 2H); 7.34 (d, 2H); 6.94 (d, 1H); 6.84 (d, 1H); 4.54 (s, 2H); 3.69-3.54 (m, 4H); 3.5-3.35 (m, 4H); 3.32 (m, 2H); 3.10 (m, 2H); 2.85 (s, 3H).

### Example 33

### 6-{2-[4-(7-fluoro-2-methylquinolin-5-yl)piperazin-1-yl]ethyl}-4H-benzo[1,4]oxazin-3-one hydrochloride salt (E33)

The title compound (E33) was prepared in 30% yield according to the general procedure starting from 7-fluoro-2-methyl-5-piperazin-1-ylquinoline (D30) and 6-(2-chloroethyl)-4H-benzo[1,4]oxazin-3-one (D4).

¹H-NMR (500 MHz, DMSO) δ: 11.41 (s, 1H); 10.84 (s, 1H); 8.79 (d, 1H); 7.70 (d, 1H); 7.68 (d, 2H); 7.35 (d, 2H); 6.93 (d, 2H); 6.85 (d, 2H); 6.80 (s, 1H), 4.54 (s, 2H); 3.69-3.54 (m, 4H); 3.5-3.35 (m, 4H); 3.35 (m, 2H); 3.05 (m, 2H); 2.86 (s, 3H).

### Example 34

### 7-Fluoro-6-{2-[4-(2-methylquinolin-5-yl)piperazin-1-yl]ethanoyl}-4H-benzo[1,4]oxazin-3-one (E34)

To a stirred suspension of 2-methyl-5-piperazin-1-ylquinoline (D3) (0.1 g, 0.45 mmol) and 6-(2-chloroethanoyl)-7-fluoro-4H-benzo[1,4]oxazin-3-one (D23) (0.14 g, 0.585 mmol) in dry acetonitrile (6 mL), diisopropylethylamine (2 mL) was added. The mixture was refluxed for 7 h, then quenched with a saturated aq. solution of NH₄Cl (10 mL) and extracted with ethy acetate (3×10 mL). The organic phase was dried (sodium sulfate) and the solvent evaporated under vacuum. The residue was purified on SPE silica cartridge (DCM/methanol 95:5) to afford compound (E34) (0.058 g, 23%).

¹H-NMR (500 MHz, DMSO) δ: 10.9 (s, 1H); 8.4 (d, 1H); 7.6 (m, 2H), 7.4 (d, 1H); 7.4 (d, 1H); 7.1 (m, 1H); 7.0 (d, 1H); 4.7 s, 2H); 3.8 (m, 2H); 3.1-2.8 (m, 8H); 2.6 (s, 3H).

### Example 35

### 6-{1-Hydroxy-2-[4-(2-methylquinolin-5-yl)piperazin-1-yl]ethyl}-4H-benzo[1,4]-oxazin-3-one (E35)

To a stirred suspension of 7-fluoro-6-{2-[4-(2-methylquinolin-5-yl)piperazin-1-yl]ethanoyl}-4H-benzo[1,4]oxazin-3-one (E34) (0.058 g, 0.134 mmol) in dry MeOH (5 mL) at 0° C. NaBH₄ (0.02 g, 4 eq) was added. The reaction mixture was stirred at 0° C. for 5 h., then quenched with a saturated aq. solution of NH₄Cl (10 mL) and extracted with ethyl acetate (3×10 mL). The combined organics were dried over (sodium sulfate), the solvent removed under vacuum and the crude purified on SPE silica cartridge (DCM to DCM/MeOH 95:5) to afford the title compound (E35) (0.028 g, 48%).

MS; (ES) m/z: [MH]⁺. 419.2

¹H-NMR (500 MHz, DMSO) δ: 10.87 (s, 1H); 10.30 (s, 1H); 8.78 (s, 1H); 7.86 (s, 2H); 7.7 (s, 1H); 7.37 (s, 1H); 7.09 (d, 1H); 4.98 (m, 1H); 6.43 (s, 1H); 5.35 (m, 1H); 4.60 (s, 2H); 3.8-3.3 (m, 8H); 2.83 (s, 3H).

### Example 36

### 6-{1-Methoxy-3-[4-(2-methylquinolin-5-yl)piperazin-1-yl]propyl}-4H-benzo[1,4]-oxazin-3-one (E36)

To a stirred suspension of 6-{1-hydroxy-3-[4-(2-methylquinolin-5-yl)-piperazin-1-yl]-propyl}-4H-benzo-[1,4]oxazin-3-one (E25) (50 mg, 0.12 mmol) in dry methanol (2 mL), trifluoroacetic acid (2 mL) was added. The reaction mixture was stirred at r.t. for 2 weeks. The solvent was concentrated in vacuo and the resulting crude product was passed through a SCX cartridge, then purified by flash chromatography, eluting with 5% methanol in DCM, to afford the title compound (E36) as a white solid (9 mg, yield 17%).

MS; (ES) m/z: 447 [MH⁺]. C₂₆H₃₀N₄O₃ requires 446.

¹H-NMR (300 MHz, DMSO) δ: 10.69 (s, 1H), 8.31 (d, 1H), 7.60 (m, 2H), 7.36 (d, 1H), 7.09 (d, 1H), 6.93-6.85 (m, 3H), 4.55 (s, 2H), 4.15 (t, 1H), 3.09 (s, 3H), 3.00 (s, 4H), 2.66-2.39 (s+m, 7H), 2.36 (q, 2H), 1.90-1.68 (m+m, 2H).

### Example 37

### 6-{2-[4-(2-Methyl-1H-indol-4-yl)piperazin-1-yl]-ethyl}-4H-benzo-[1,4]oxazin-3-one hydrochloric acid salt (E37)

The title compound (E37) was prepared in 26% yield according to the general procedure from 2-methyl-4-piperazin-1-yl-1H-indole.

MS; (ES) m/z: 391.2 [MH⁺].

¹H-NMR (500 MHz, DMSO) δ: 10.59 (s, 1H), 10.78 (s, 1H); 10.65 (s, 1H); 6.97 (d, 1H); 6.92 (t, 1H); 6.90 (d, 1H); 6.84 (dd, 1H); 6.78 (d, 1H); 6.46 (d, 1H); 6.12 (s, 1H); 4.54 (s, 2H); 3.66 (t, 2H); 3.65 (m, 2H); 3.3 (m, 4H); 3.10 (t, 2H); 3.0 (m, 2H); 2.35 (s, 3H).

### Example 38

### 6-{2-[4-(5,6,7,8-Tetrahydronaphthalen-1-yl)piperazin-1-yl]ethyl}-4H-benzo-[1,4]oxazin-3-one (E38)

The title compound (E38) was prepared in 84% yield according to the general procedure from 1-(5,6,7,8-tetrahydronaphthalen-1-yl)piperazine.

MS; (ES) m/z: 392.2 [MH⁺].

¹H-NMR (500 MHz, DMSO) δ: 10.79 (s, 1H); 10.64 (s, 1H); 7.08 (t, 1H); 6.92 (d, 1H); 6.85 (dd, 1H); 6.83 (m, 2H); 6.78 (d, 1H); 4.54 (s, 2H); 3.58 (d, 2H); 3.29 (m, 2H); 3.15 (d, 2H); 3.19 (m, 2H); 3.06 (m, 2H); 2.99 (m, 2H); 2.72 (m, 2H); 2.66 (m, 2H); 1.71, m, 4H).

### Example 39

### 6-[2-(4-Naphthalen-1-ylpiperazin-1-yl)ethyl]-4H-benzo[1.4]oxazin-3-one hydrochloride salt (E39)

The title compound (E39) was prepared in 82% yield following the general procedure described above from 1-napthalen-1-ylpiperazine.

MS; (ES) m/z: 388.3 [MH⁺]. C₂₄H₂₅N₃O₂ requires 387.49

¹H NMR (500 MHz, DMSO) δ: δ: 10.79 (s, 1H), 10.64 (bs, 1H), 8.13 (m, 1H), 7.93 (m, 1H), 7.68 (d, 1H), 7.54 (m, 2H), 7.48 (t, 1H), 7.21 (d, 1H), 6.96 (d, 1H), 6.87 (dd, 1H), 6.81 (d, 1H), 4.56 (s, 2H), 3.68-3.46 (m/m, 2/2H), 3.46-3.21 (m/m, 2/2H), 3.38 (t, 2H), 3.03 (t, 2H).

### Example 40

### 6-{1-Fluoro-2-[4-(2-methylquinolin-5-yl)piperazin-1-yl]ethyl}-4H-benzo[1,4]-oxazin-3-one (E40)

A stirred suspension of (E13 free base) (100 mg, 0.24 mmol, 1.0 eq.) in dry DCM (4 ml) was cooled to 0° and DAST (48 uL, 0.36 mmol, 1.5 eq) was added dropwise. The reaction mixture was stirred at 0° under nitrogen for 1 h and at r.t. overnight. The reaction was quenched with a saturated aq. solution of sodium carbonate (20 mL) and extracted into dichloromethane (3×15 mL). The organic layers were combined, dried (Na₂SO₄) and concentrated in vacuo. The crude product was purified by flash chromatography, eluting with 2% methanol in DCM to give the title compound (E40) as a yellow solid (75 mg, 75%).

MS; (ES) m/z: 421 [MH⁺]. C₂₄H₂₅FN₄O₂ requires 420.

¹H-NMR (500 MHz, DMSO) δ: 10.76 (s, 1H), 8.32 (d, 1H), 7.57 (m, 2H), 7.37 (d, 1H), 7.10 (dd, 1H), 6.95 (m, 3H), 5.55 (m, 1H), 4.60 (s, 2H), 2.90 (s, 2H), 3.03 (m, 4H), 2.81 (m, 4H), 2.62 (s, 3H).

### Example 41

### 6-{1-Fluoro-3-[4-(2-methylquinolin-5-yl)piperazin-1-yl]propyl}-4H-benzo[1,4]-oxazin-3-one (E41)

A stirred suspension of (E25) (50 mg, 0.116 mmol, 1.0 eq.) in dry DCM (2 ml) was cooled to 0° and DAST (23 uL, 0.174 mmol, 1.5 eq) was added dropwise. The reaction mixture was stirred at 0° under nitrogen for 1 h and at r.t. overnight. The reaction was quenched with a saturated aq. solution of sodium carbonate (10 mL) and extracted into DCM (3×15 mL). The organic layers were combined, dried (Na₂SO₄) and concentrated in vacuo. The crude product was purified by flash chromatography, eluting with 2% methanol in dichloromethane to give the title compound (E41) as a yellow solid (19 mg, 38%).

MS; (ES) m/z: 435 [MH⁺]. C₂₅H₂₇FN₄O₂ requires 434.

¹H-NMR (300 MHz, DMSO) δ: 10.74 (s, 1H), 8.32 (d, 1H), 7.57 (m, 2H), 7.37 (d, 1H), 7.10 (dd, 1H), 6.95 (m, 3H), 5.55 (m, 1H), 4.57 (s, 2H), 3.02 (m, 4H), 2.65 (m, 4H), 2.60 (s, 3H), 2.50 (m, 2H), 2.40-1.90 (m, 2H).

### Example 42

### 5-Fluoro-6-{2-[4-(2-methylquinolin-5-yl)piperazin-1-yl]ethyl}-4H-benzo[1.4]-oxazin-3-one (E42)

6-(2-Chloroethyl)-5-fluoro-4H-benzo[1,4]oxazin-3-one (D41) (0.105 g, 0.46 mmol), sodium iodide (0.068 g, 0.46 mmol) and sodium carbonate (0.048 g, 0.46 mmol) were added to a solution of 2-methyl-5-piperazin-1-yl-quinoline (D3) (0.08 g, 0.35 mmol) in NMP (2.5 mL) at room temperature. The suspension was heated to 120° C. for 3 h under nitrogen, then diluted with ethyl acetate (20 mL) and washed with water (2×15 mL). The combined aqueous layers were back extracted with ethyl acetate (15 mL) and the combined organic layers were then washed with brine (20 mL) and dried over anhydrous Na₂SO₄. Removal of the organic solvent under reduced pressure gave a crude which was purified by flash chromatography eluting with DCM, then 2-5% methanol in DCM, affording the title compound (E42) as a white solid (0.068 g, 46%).

MS; (ES) m/z 421.4 [MH]⁺. C₂₄H₂₅FN₄O₂ requires 420.

¹H-NMR (500 MHz, CDCl₃) δ: 8.20 (d, 1H), 7.80 (bs, 1H), 7.70 (d, 1H), 7.59 (t, 1H) 7.20 (d, 1H), 7.10 (d, 1H), 6.80 (t, 1H), 6.70 (d, 1H) 4.50 (s, 2H), 3.12 (m, 4H), 2.90 (m, 4H), 2.70 (s, 3H), 2.65 (m, 4H).

### Example 43

### 5-Fluoro-4-methyl-6-{2-[4-(2-methylquinolin-5-yl)piperazin-1-yl]ethyl}-4H-benzo[1,4]-oxazin-3-one (E43)

6-(2-Chloroethyl)-5-fluoro-4-methyl-4H-benzo[1,4]oxazin-3-one (D42) (0.080 g, 0.33 mmol), sodium iodide (0.049 g, 0.33 mmol) and sodium carbonate (0.035 g, 0.33 mmol) were added to a solution of 2-methyl-5-piperazin-1-yl-quinoline (D3) (0.097 g, 0.43 mmol) in NMP (2.5 mL) at room temperature. The suspension was heated to 120° C. for 3 h under nitrogen, then diluted with ethyl acetate (20 mL) and washed with water (2×15 mL). The combined aqueous layers were back extracted with ethyl acetate (15 mL) and the combined organic layers were then washed with brine (20 mL) and dried (Na₂SO₄). Removal of the organic solvent under reduced pressure gave a crude which was purified by flash chromatography eluting with DCM then 2% methanol in DCM to afford the title compound as a white solid (0.078 g, yield 55%).

MS; (ES) m/z 435.5 [MH]⁺. C₂₅H₂₇FN₄O₂ requires 434.

¹H-NMR (500 MHz, CDCl₃) δ: 8.35 (d, 1H), 7.70 (bs, 1H), 7.55 (t, 1H), 7.25 (d, 1H) 7.05 (d, 1H), 6.90 (t, 1H), 6.75 (dd, 1H), 6.70 (d, 1H) 4.50 (s, 2H), 3.47 (d, 3H), 3.15 (m, 4H), 2.80 (m, 6H), 2.70 (m, 2H), 2.70 (s, 3H).

### Example 44

### 6-{2-[4-(7-Chloro-2-methylquinolin-5-yl)piperazin-1-yl]ethyl}-4-methyl-4H-benzo-[1,4]-oxazin-3-one (E44)

The title compound (E44) was prepared according to the general procedure from 7-chloro-2-methyl-5-piperazin-1-ylquinoline (D9) and 6-(2-chloroethyl)-4-methyl-4H-benzo[1,4]oxazin-3-one (D43).

MS; (ES) m/z: 451.9 [MH]⁺. C₂₅H₂₇ClN₄O₂.HCl requires 450.

¹H-NMR (500 MHz, DMSO) δ: 11.20 (bs, 1H), 8.70 (d, 1H), 7.92 (s, 1H), 7.69 (d, 1H), 7.35 (s, 1H), 7.13 (d, 1H), 6.96 (m, 2H), 4.63 (s, 3H), 3.80-3.30 (m, 10H), 3.30 (s, 3H), 2.82 (s, 3H).

### Example 45

### 4-Ethyl-6-{2-[4-(2-methylquinolin-5-yl)piperazin-1-yl]ethyl}-4H-benzo[1,4]-oxazin-3-one hydrochloride salt (E45)

The title compound (E45) was prepared according to the general procedure from 2-methyl-5-piperazin-1-ylquinoline (D3) and 6-(2-chloroethyl)-4-ethyl-4H-benzo-[1,4]oxazin-3-one (D44). The free base was dissolved in DCM, HCl (1M solution in Et₂O) was added and the yellow solid thus obtained was washed with diethyl ether to give the HCl salt.

MS; (ES) m/z: 431 [MH]⁺. C₂₆H₃₀N₄O₂ requires 430.

¹H-NMR of HCl salt (500 MHz, DMSO) δ: 10.69 (bs, 1H), 8.79 (bs, 1H), 8-7.7 (3bs, 3H), 7.35 (bs, 1H), 7.16 (d, 1H), 7.00 (d, 1H), 6.96 (dd, 1H), 4.61 (s, 2H), 3.95 (q, 2H), 3.7 (m, 2H), 3.5-3.2 (m, 8H+H2O), 3.10 (dd, 2H), 2.83 (bs, 3H), 1.19 (t, 3H).

### Example 46

### 6-{2-[4-(7-Fluoro-2-methylquinolin-5-yl)piperazin-1-yl]ethyl}-4-methyl-4H-benzo-[1,4]-oxazin-3-one hydrochloride salt (E46)

The title compound (E46) was prepared according to the general procedure from 7-fluoro-2-methyl-5-piperazin-1-ylquinoline (D30) and 6-(2-chloroethyl)-4-methyl-4H-benzo[1,4]oxazin-3-one (D43). The product was dissolved in DCM, HCl (1M solution in Et₂O) was added and the yellow solid thus obtained was washed with diethyl ether to give the HCl salt.

MS; (ES) m/z: 435 [MH]⁺. C₂₅H₂₇FN₄O₂ requires 434.

¹H-NMR of HCl salt (500 MHz, DMSO) δ: 10.69 (bs, 1H), 8.56 (bs, 1H), 7.57 (bs, 1H), 7.48 (bs, 1H, 7.25 (bs, 1H), 7.12 (d, 1H), 6.98 (d, 1H), 6.95 (dd, 1H), 4.63 (s, 2H), 3.7 (bm, 10H), 3.3 (s, 3H), 3.09 (dd, 2H), 2.74 (bs, 3H).

### Example 47

### 6-{1-(Methyloxy)-2-[4-(2-methyl-5-quinolinyl)-1-piperazinyl]ethyl}-2H-1,4-benzoxazin-3(4H)-one (E47)

To a solution of 6-{1-hydroxy-2-[4-(2-methylquinolin-5-yl)piperazin-1-yl]ethyl}-4H-benzo[1.4]oxazin-3-one (E13) (30 mg, 0.07 mmol, 1.0 eq) in methanol (1 ml) three portions of p-toluenesulfonic acid was added (each portion: 27 mg, 0.14 mmol, 2.0 eq). After each addition the mixture was heated at 90° C. in a microwave oven (2×30 min). The crude reaction mixture was passed through a SCX cartridge, then purified by flash chromatography, eluting with 3% methanol in DCM, to afford the title compound (E47) as a yellow solid (20 mg, yield 66%).

MS; (ES) m/z: 433 [MH⁺]. C₂₅H₂₈N₄O₃ requires 432.

¹H-NMR (500 MHz, DMSO) δ: 10.7 (bs, 1H), 8.32 (d, 1H), 7.58 (m, 2H), 7.35 (d, 1H), 7.08 (dd, 1H), 6.95-6.85 (m, 2H), 4.56 (s, 2H), 4.35 (m, 1H), 3.13 (s, 3H), 2.69/2.45 (m/m, 1/1H), 2.99 (bm, 4H), 2.75 (bm, 4H), 2.62 (s, 3H)

### Example 48

### 6-{1-Amino-2-[4-(2-methyl-5-quinolinyl)-1-piperazinyl]ethyl}-2H-1,4-benzoxazin-3(4H)-one (E48)

To a solution of 6-{2-[4-(2-methylquinolin-5-yl)piperazin-1-yl]ethanoyl}-4H-benzo[1.4]oxazin-3-one (E12) (80 mg, 0.19 mmol, 1.0 eq) in methanol (2 ml), ammonium acetate (146 mg, 1.9 mmol, 10 eq) and sodium cyanoborohydride (36 mg, 0.57 mmol, 3 eq) were added. The mixture was heated at 90° C. in a microwave oven for 20 min. The solvent was concentrated in vacuo, the residue was partitioned between water (20 ml) and DCM (3×30 ml) and the combined organic phases dried (Na₂SO₄) and concentrated in vacuo, to afford the title compound (E48) as a yellow solid (42 mg, yield 53%).

MS; (ES) m/z: 418 [MH⁺]. C₂₄H₂₇N₅O₂ requires 419.

¹H-NMR (500 MHz, DMSO) δ: 10.64 (s, 1H), 8.31 (d, 1H), 7.56 (m, 2H), 7.35 (d, 1H), 7.09 (dd, 1H), 6.98 (d, 1H), 6.91 (dd, 1H), 6.86 (d, 1H), 4.50 (s, 2H), 4.03 (m, 1H), 3.02 (bs, 4H), 2.80 (bs, 4H), 2.60 (bs, 2H), 2.61 (s, 3H), 2.4-2.3 (m, 2H), 1.90 (bs, 2H)

### Example 49

### N-[2-[4-(2-Methyl-5-quinolinyl)-1-piperazinyl]-1-(3-oxo-3,4-dihydro-2H-1,4-benzoxazin-6-yl)ethyl]acetamide hydrochloride (E49)

To a solution of 6-{2-[4-(2-methylquinolin-5-yl)piperazin-1-yl]ethanoyl}-4H-benzo[1.4]oxazin-3-one (E12) (70 mg, 0.17 mmol, 1.0 eq) in methanol (3 ml), ammonium acetate (131 mg, 1.7 mmol, 10 eq) and sodium cyanoborohydride (32 mg, 0.51 mmol, 3 eq) were added. The mixture was heated at 90° C. in a microwave oven for 45 min. The solvent was concentrated in vacuo, the residue was dissolved in saturated NH₄Cl and extracted with DCM and the organic phases dried (Na₂SO₄) and concentrated in vacuo. The crude compound was purified by MS directed prep-HPLC. The resulting product was dissolved in methanol (2 ml) and treated with 1.25M HCl in ethanol (30 ul). The mixture was stirred at r.t. for 0.5 h, then concentrated in vacuo to give the title compound (E49) as a yellow solid (10 mg, yield 12%). ¹H-NMR (500 MHz, DMSO) δ: 10.48 (s, 1H), 8.48 (d, 1H), 8.38 (bs, 1H), 7.69 (m, 2H), 7.46 (d, 1H), 7.22 (dd, 1H), 7.0-6.85 (m, 3H), 5.23 (m, 1H), 4.50 (s, 2H), 2.68 (s, 3H), 1.91 (s, 3H)

### Example 50

### 6-{1-(Methylamino)-2-[4-(2-methyl-5-quinolinyl)-1-piperazinyl]ethyl}-2H-1,4-benzoxazin-3(4H)-one (E50)

To a solution of 6-{2-[4-(2-methylquinolin-5-yl)piperazin-1-yl]ethanoyl}-4H-benzo[1.4]oxazin-3-one (E12) (80 mg, 0.19 mmol, 1.0 eq) in methanol (2 ml) three portions of methylamine hydrochloride (each portion: 90 mg, 1.33 mmol, 7.0 eq) and sodium cyanoborohydride (each portion: 48 mg, 0.76 mmol, 4 eq) were added. After each addition the mixture was heated at 100° C. in a microwave oven for 20 min+40 min+90 min. The solvent was concentrated in vacuo and the residue dissolved in saturated aqueous NaHCO₃ (20 ml) and extracted with DCM (3×30 ml). The organic phases were dried (Na₂SO₄) and concentrated in vacuo. The crude compound was purified by MS directed prep-HPLC to afford the title compound (E50) as a yellow solid (36 mg, yield 44%).

MS; (ES) m/z: 432 [MH⁺]. C₂₅H₂₉N₅O₂ requires 431. ¹H-NMR (500 MHz, DMSO) δ: 10.64 (s, 1H), 8.30 (d, 1H), 7.56 (m, 2H), 7.35 (d, 1H), 7.09 (dd, 1H), 6.92-6.88 (m, 3H), 4.52 (s, 2H), 3.58 (dd, 1H), 3.02 (bm, 4H), 2.77 (bm, 2H), 2.60 (bm, 2H), 2.61 (s, 3H), 2.42 (d, 1H), 2.28 (dd, 1H), 2.15 (s, 3H)

### Example 51

### 6-[2-[4-(2-Methyl-5-quinolinyl)-1-piperazinyl]-1-(phenyloxy)ethyl]-2H-1,4-benzoxazin-3(4H)-one (E51)

To a solution of 6-{1-amino-2-[4-(2-methyl-5-quinolinyl)-1-piperazinyl]ethyl}-2H-1,4-benzoxazin-3(4H)-one (E48) (45 mg, 0.12 mmol, 1.0 eq) in phenyl ether (2 ml) phenol (226 mg, 2.4 mmol, 20 eq) and p-toluenesulfonic acid (91 mg, 0.48 mmol, 4.0 eq) were added. The mixture was heated at 130° C. in a microwave oven (30+15 min). The crude reaction mixture was passed through a SCX cartridge, then purified by flash chromatography, eluting with 3% methanol in DCM, to afford the title compound (E51) as a yellow solid (14 mg, yield 24%).

MS; (ES) m/z: 495 [MH⁺]. C₃₀H₃₀N₄O₃ requires 494. ¹H-NMR (500 MHz, DMSO) δ: 10.56 (bs, 1H), 8.30 (d, 1H), 7.55 (m, 2H), 7.36 (d, 1H), 7.05 (m, 3H), 6.84 (bs, 3H), 6.78 (bs, 1H), 4.49 (s, 2H), 4.11 (bt, 1H), 2.93 (bs, 6H), 2.71 (bs, 4H), 2.61 (s, 3H).

### Example 52

### [2-[4-(2-Methyl-5-quinolinyl)-1-piperazinyl]-1-(3-oxo-3,4-dihydro-2H-1,4-benzoxazin-6-yl)ethyl]formamide (E52)

To a solution of 6-{1-amino-2-[4-(2-methyl-5-quinolinyl)-1-piperazinyl]ethyl}-2H-1,4-benzoxazin-3(4H)-one (E48) (180 mg, 0.48 mmol, 1.0 eq) in DMF (4 ml) dimethylamine hydrochloride (391 mg, 4.8 mmol, 10.0 eq) was added. The mixture was heated at 100° C. in a microwave oven for 30 min. The solvent was concentrated in vacuo. The residue was dissolved in saturated aqueous NaHCO₃ (20 ml) and extracted with DCM (3×30 ml). The organic phases were separated, dried (Na₂SO₄) and concentrated in vacuo. The crude product was passed through a SCX cartridge, then purified by flash chromatography, eluting with 5% methanol in DCM, to afford the title compound (E52) as a yellow solid (49 mg, yield 23%).

MS; (ES) m/z: 446 [MH⁺]. C₂₅H₂₇N₅O₃ requires 445. ¹H-NMR (300 MHz, DMSO) δ: 10.7 (bs, 1H), 8.5 (d, 1H), 8.4 (d, 1H), 8.1 (s, 1H), 7.6 (m, 2H), 7.4 (d, 1H), 7.1 (m, 1H), 6.9 (m, 3H), 5.0 (m, 1H), 4.6 (s, 1H), 3.0 (bs, 4H), 2.9-2.7 (m, 6H), 2.6 (s, 3H)

### Example 53

### 6-{1-Hydroxy-1-methyl-3-[4-(2-methyl-5-quinolinyl)-1-piperazinyl]propyl}-2H-1,4-benzoxazin-3(4H)-one (E53)

A solution of methylmagnesium bromide 3M in ether (230 uL, 0.69 mmol, 3 eq) was added dropwise to a suspension of 6-{3-[4-(2-methylquinolin-5-yl)-piperazin-1-yl]-propanoyl}-4H-benzo[1,4]-oxa-zin-3-one (E24) (100 mg, 0.23 mmol, 1.0 eq.) in dry THF (3 ml) cooled at 0° C. The mixture was stirred at 0° C. for 2 h then quenched with saturated aqueous NH₄Cl (10 ml) and extracted with dichloromethane (3×15 ml). The organic phases were dried (Na₂SO₄) and concentrated in vacuo. The crude product was triturated with ether to afford the title compound (E53) as a white solid (62 mg, yield 60%).

MS; (ES) m/z: 447 [MH⁺]. C₂₆H₃₀N₄O₃ requires 446

1H-NMR (500 MHz, DMSO) δ: 10.60 (s, 1H), 8.30 (d, 1H), 7.57 (m, 2H), 7.36 (d, 1H), 7.08 (dd, 1H), 7.06 (d, 1H), 6.94 (dd, 1H), 6.86 (d, 1H), 5.69 (s, 1H), 4.52 (s, 2H), 2.98 (m, 4H), 2.7-2.5 (m, 4H), 2.61 (s, 3H), 2.45-2.25 (m, 2H), 1.87 (m, 2H), 1.38 (s, 3H)

### Example 54

### 6-{1-Hydroxy-1-methyl-2-[4-(2-methyl-5-quinolinyl)-1-piperazinyl]ethyl}-2H-1,4-benzoxazin-3(4H)-one (E54)

A solution of methylmagnesium bromide 3M in ether (240 uL, 0.72 mmol, 3 eq) was added dropwise to a suspension of 6-{2-[4-(2-methylquinolin-5-yl)piperazin-1-yl]ethanoyl}-4H-benzo[1.4]oxazin-3-one (E12) (100 mg, 0.24 mmol, 1.0 eq.) in dry THF (3 ml) cooled at 0° C. The mixture was stirred at 0° C. for 2 h then quenched with saturated aqueous NH₄Cl (10 ml) and extracted with DCM (3×15 ml). The combined organic phases were dried (Na₂SO₄) and concentrated in vacuo. The crude product was purified by flash chromatography, eluting with 5% methanol in DCM, to afford the title compound (E54) as a white solid (62 mg, yield 60%).

MS; (ES) m/z: 433 [MH⁺]. C₂₅H₂₈N₄O₃ requires 432

1H-NMR (500 MHz, DMSO) δ: 10.63 (s, 1H), 8.28 (d, 1H), 7.56 (m, 2H), 7.34 (d, 1H), 7.07 (dd, 1H), 7.10 (d, 1H), 6.99 (dd, 1H), 6.86 (d, 1H), 4.85 (s, 1H), 4.51 (s, 2H), 2.94 (m, 4H), 2.7-2.5 (m, 4H), 2.61 (s, 3H), 2.60 (dd, 2H), 1.38 (s, 3H)

### Example 55

### 6-{(1E)-1-Methyl-3-[4-(2-methyl-5-quinolinyl)-1-piperazinyl]-1-propen-1-yl}-2H-1,4-benzoxazin-3(4H)-one (E55)

A stirred suspension of 6-{1-hydroxy-1-methyl-2-[4-(2-methyl-5-quinolinyl)-1-piperazinyl]ethyl}-2H-1,4-benzoxazin-3(4H)-one (E54) (48 mg, 0.11 mmol, 1.0 eq.) and p-toluenesulfonic acid (105 mg, 0.55 mmol, 5 eq) in dry toluene (3 ml) was refluxed for 6 h. The mixture was quenched with saturated aqueous NaHCO₃ (5 ml) and extracted with ethyl acetate (3×15 ml). The combined organic phases were dried (Na₂SO₄) and concentrated in vacuo. The crude product was purified by flash chromatography, eluting with 2% methanol in DCM, to afford the title compound (E55) (20 mg, yield 42%).

MS; (ES) m/z: 429 [MH⁺]. C₂₆H₂₈N₄O₂ requires 428

1H-NMR (500 MHz, DMSO) δ: 10.58 (bs, 1H), 8.30 (d, 1H), 7.54 (m, 2H), 7.33 (d, 1H), 7.06 (dd, 1H), 6.95 (m, 2H), 6.85 (d, 1H), 5.75 (t, 1H), 4.50 (s, 2H), 3.18 (d, 2H), 2.99 (m, 4H), 2.67 (m, 4H), 2.58 (s, 3H), 1.97 (s, 3H).

### Example 56

### 6-(1-{2-[4-(2-Methyl-5-quinolinyl)-1-piperazinyl]ethyl}ethenyl)-2H-1,4-benzoxazin-3(4H)-one (E56)

A solution of Tebbe Reagent 0.5M in toluene (1.0 mL, 0.506 mmol, 2.2 eq) was added dropwise to a solution of 6-{3-[4-(2-methylquinolin-5-yl)-piperazin-1-yl]-propanoyl}-4H-benzo[1,4]-oxa-zin-3-one (E24) (100 mg, 0.23 mmol, 1.0 eq.) in dry THF (3 ml) at 0° C. The mixture was stirred at r.t. overnight then quenched with saturated aqueous NH₄Cl (5 ml) and extracted with DCM (3×10 ml). The combined organics were dried (Na₂SO₄) and concentrated in vacuo. The crude product was dissolved in methanol and passed through a SCX cartridge, then purified by flash chromatography, eluting with 2% methanol in DCM, to afford the title compound (E56) (26 mg, yield 26%).

MS; (ES) m/z: 429 [MH⁺]. C₂₆H₂₈N₄O₂ requires 428

1H-NMR (500 MHz, DMSO) δ: 10.67 (s, 1H), 8.33 (d, 1H), 7.58 (m, 2H), 7.38 (d, 1H), 7.10 (d, 1H), 7.04 (dd, 1H), 7.0 (d, 1H), 6.91 (d, 1H), 5.24-5.08 (d/d, 2H), 4.57 (s, 2H), 3.01 (bm, 2H), 2.66 (bm, 2H), 3.01-2.66 (m/m, 4H), 2.66-2.5 (m/m, 4H), 2.64 (s, 3H)

### Example 57

### 6-(1-{[4-(2-Methyl-5-quinolinyl)-1-piperazinyl]methyl}ethenyl)-2H-1,4-benzoxazin-3(4H)-one (E57)

A solution of Tebbe Reagent 0.5M in toluene (2.2 mL, 1.1 mmol, 2.2 eq) was added dropwise to a solution of 6-{2-[4-(2-methylquinolin-5-yl)piperazin-1-yl]ethanoyl}-4H-benzo[1.4]oxazin-3-one (E12) (200 mg, 0.48 mmol, 1.0 eq.) in dry THF (6 ml) cooled at 0° C. The mixture was stirred at r.t. overnight then quenched with saturated aqueous NH₄Cl (5 ml) and extracted with ethyl acetate (4×15 ml). The combined organic phases were dried (Na₂SO₄) and concentrated in vacuo to afford the crude product which was purified by flash chromatography, eluting with 2% methanol in DCM, to afford the title compound (E57) (35 mg, yield 18%).

MS; (ES) m/z: 415 [MH⁺]. C₂₅H₂₆N₄O₂ requires 414

1H-NMR (500 MHz, DMSO) δ: 10.70 (s, 1H), 8.33 (d, 1H), 7.57 (m, 2H), 7.38 (d, 1H), 7.16 (m, 2H), 7.08 (dd, 1H), 6.90 (d, 1H), 5.43 (s, 1H), 5.22 (s, 1H), 4.56 (s, 2H), 3.38 (s, 2H), 3.00-2.69 (bs, 4H), 2.69-2.50 (bs, 4H), 2.62 (bs, 3H)

### Example 58

### 2-[4-(2-Methyl-5-quinolinyl)-1-piperazinyl]-1-(3-oxo-3,4-dihydro-2H-1,4-benzoxazin-6-yl)ethyl acetate (E58)

To a solution of 6-{1-hydroxy-2-[4-(2-methylquinolin-5-yl)piperazin-1-yl]ethyl}-4H-benzo[1.4]oxazin-3-one (E13) (60 mg, 0.14 mmol, 1.0 eq) in dry DCM (5 ml) 4-(dimethylamino)pyridine (21 mg, 0.17 mmol, 1.2 eq) was added. The solution was stirred at room temperature for 72 h whilst three portion of acetic anhydride was added (each portion: 16 uL, 0.17 mmol, 1.2 eq). The mixture quenched with water (10 ml) and extracted with DCM (3×15 ml). The combined organic phases were dried (Na₂SO₄) and concentrated in vacuo to afford the crude product which was purified by MS directed prep-HPLC, to afford the title compound (E58) as a yellowish solid (34 mg, yield 52%).

MS; (ES) m/z: 461 [MH⁺]. C₂₆H₂₈N₄O₄ requires 460.

¹H-NMR (500 MHz, DMSO) δ: 10.66 (s, 1H), 8.28 (d, 1H), 7.55 (m, 2H), 7.33 (d, 1H), 7.05 (m, 1H), 6.95-6.85 (m, 3H), 5.80 (m, 1H), 4.52 (s, 2H), 2.95 (bs, 4H), 2.82 (dd, 1H), 2.8-2.6 (bm, 4H), 2.58 (s, 3H), 2.58 (dd, 1H), 2.02 (s, 3H).

### Examples 59 and 60

### 6-{1-Hydroxy-2-[4-(2-methyl-5-quinolinyl)-1-piperazinyl]ethyl}-2H-1,4-benzoxazin-3(4H)-one hydrochloride

### Separation of Enantiomers (E59 & E60)

Racemic 6-{1-hydroxy-2-[4-(2-methylquinolin-5-yl)piperazin-1-yl]ethyl}-4H-benzo[1.4]-oxazin-3-one (E13) (100 mg, 0.24 mmol) was subjected to chiral separation using preparative HPLC (Chiralcel OD 25 cm×20 mm, elution with 20% ethanol in hexane). The resulting individual enantiomers of unknown stereochemistry were dissolved in methanol (3 ml) and treated with 1.25 M HCl in ethanol (0.5 ml) followed by evaporation in vacuo to afford the title compounds (E59) (45 mg) and (E60) (43 mg).

Enantiomeric purity was verified by analytical chiral HPLC (Chiralcel OD 25 cm×4.6 mm, elution with 15% ethanol in hexane):

(E59), Rt=21.5 min, 99% A/A %
(E60), Rt=25.6 min, 99% A/A %

### Example 61

### 6-{[4-(8-Quinolinyl)-1-piperazinyl]methyl}-2H-1,4-benzoxazin-3(4H)-one hydrochloride (E61)

A solution of 8-(1-piperazinyl)quinoline (56 mg; 0.26 mmol; 1 eq) and 3-oxo-3,4-dihydro-2H-1,4-benzoxazine-6-carbaldehyde (47 mg; 0.26 mmol; 1 eq) in dichloroethane (2.5 ml) was stirred at rt for 30 min. Acetic acid (0.015 ml; 0.26 mmol; 1 eq) and sodium triacetoxyborohydride (110 mg; 0.52 mmol; 2 eq) were added and the reaction mixture was stirred overnight. Water (10 ml) was added to the reaction mixture and the mixture extracted with DCM (3×10 ml). The organic phases were combined, dried (Na₂SO₄) and concentrated in vacuo to afford the crude product which was purified on SPE cartridge (Si; 1 g) eluting with a gradient of 1 to 5% MeOH in DCM to give the free base of the title compound (E61) (52 mg; 53%).

### Free Base Analytical Data:

MS; (ES) m/z: 375.4 [MH⁺]. C₂₂H₂₂N₄O₂ requires 374.4.

¹H NMR (500 MHz, CDCl₃) δ: 8.88 (m, 1H), 8.12 (dd, 1H), 7.88 (bs, 1H), 7.45 (m, 2H), 7.37 (m, 1H), 7.14 (m, 1H), 7.0-6.9 (m, 3H), 4.62 (s, 2H), 3.62 (s, 2H), 3.48 (m, 4H), 2.85 (m, 4H).

The free base was converted into the hydrochloride salt by dissolving the compound in MeOH and adding 0.3 ml (2 eq) of 1M solution of HCl in MeOH. The resulting solution was stirred for 1 h then evaporated to dryness and the residue triturated with Et₂O to give of the title compound (E61) (54 mg) by filtration.

### Example 62

### 6-{2-[(1S,4S)-5-(2-Methyl-5-quinolinyl)-2,5-diazabicyclo[2.2.1]hept-2-yl]ethyl}-2H-1,4-benzoxazin-3(4H)-one (E62)

The title compound (E62) was prepared from 5-[(1S,4S)-2,5-diazabicyclo[2.2.1]hept-2-yl]-2-methylquinoline (D46) according to the general procedure for the alkylation of arylpiperazines with 6-(2-chloroethyl)-4H-benzo[1,4]oxazin-3-one (D4).

MS; (ES) m/z: 415.3 [MH⁺]. C₂₅H₂₆N₄O₂ requires 414.52.

¹H NMR (500 MHz, DMSO) δ: 1 (s, 1H), 10.74 (s, 1H), 8.93 (bs, 1H), 7.85 (t, 1H), 7.70 (m, 2H), 7.22 (d, 1H), 6.9-6.7 (m, 3H), 4.8-4.52 (bm, 2H), 4.49 (s, 2H), 3.85 (m, 2H), 3.39-3.0 (m/m, 2/2H), 3.3 (m, 2H), 2.78 (s, 3H), 2.4-2.3 (m/m, 1/1H).

### Example 63

### 6-{2-[4-(2-Quinolinyl)-1-piperazinyl]ethyl}-2H-1,4-benzoxazin-3(4H)-one (E63)

The title compound (E63) was prepared from 2-(1-piperazinyl)quinoline (D47) according to the general procedure for the alkylation of arylpiperazines with 6-(2-chloroethyl)-4H-benzo[1,4]oxazin-3-one (D4).

MS; (ES) m/z: 389.5 [MH⁺]. C₂₃H₂₄N₄O₂ requires 388.47.

¹H NMR (300 MHz, DMSO) δ: 10.6 (bs, 1H), 8.0 (d, H), 7.68 (d, 1H), 7.52 (m, 2H), 7.20 (m, 2H), 6.87-6.75 (m, 3H), 4.51 (s, 2H), 3.65 (m, 4H), 3.3 (m, 4H), 2.68 (t, 2H), 2.58 (t, 2H).

### Example 64

### 6-{3-[4-(2-Quinolinyl)-1-piperazinyl]propyl}-2H-1,4-benzoxazin-3(4H)-one (E64)

The title compound (E64) was prepared from 2-(1-piperazinyl)quinoline (D47) and 6-(3-chloropropyl)-2H-1,4-benzoxazin-3(4H)-one (D49) according to the general procedure for the alkylation of arylpiperazines.

MS; (ES) m/z: 403.5 [MH⁺]. C₂₄H₂₆N₄O₂ requires 402.5.

¹H NMR (300 MHz, DMSO) δ: 10.6 (bs, 1H), 8.0 (d, H), 7.68 (d, 1H), 7.52 (m, 2H), 7.20 (m, 2H), 6.85 (d, 1H), 6.75 (m, 2H), 4.51 (s, 2H), 3.65 (m, 4H), 3.3 (m, 4H), 2.5 (m, 2H), 2.3 (t, 2H), 1.7 (t, 2H).

### Example 65

### 6-{2-[4-(6-Chloro-2-quinolinyl)-1-piperazinyl]ethyl}-2H-1,4-benzoxazin-3(4H)-one (E65)

The title compound (E65) was prepared from 6-chloro-2-(1-piperazinyl)quinoline according to the general procedure for the alkylation of arylpiperazines with 6-(2-chloroethyl)-4H-benzo[1,4]oxazin-3-one (D4).

MS; (ES) m/z: 423.2 [MH⁺]. C₂₃H₂₃N₄O₂ requires 422.9.

¹H NMR (300 MHz, CDCl₃) δ: 7.8 (d, 1H), 7.67 (bs, 1H), 7.6 (d, 1H), 7.55 (d, 1H), 7.44 (dd, 1H), 6.98 (d, 1H), 6.7 (d, 1H), 6.83 (dd, 1H), 6.65 (s, 1H), 4.51 (s, 2H), 3.8 (m, 4H), 2.8 (m, 2H), 2.6 (m, 6H).

### Example 66

### 6-{2-[4-(6-Nitro-2-quinolinyl)-1-piperazinyl]ethyl}-2H-1,4-benzoxazin-3(4H)-one (E66)

The title compound (E66) was prepared from 6-nitro-2-(1-piperazinyl)quinoline according to the general procedure for the alkylation of arylpiperazines with 6-(2-chloroethyl)-4H-benzo[1,4]oxazin-3-one (D4).

MS; (ES) m/z: 434.5 [MH⁺]. C₂₃H₂₃N₅O₄ requires 433.47.

¹H NMR (300 MHz, DMSO) δ: 10.6 (s, 1H), 8.72 (d, 1H), 8.25 (m, 2H), 7.6 (d, 1H), 7.4 (d, 1H), 6.85-6.73 (d/d, 2H), 4.51 (s, 2H), 3.75 (m, 4H), 3.3 (m, 4H), 2.68 (t, 2H), 2.58 (t, 2H).

### Example 67

### 6-{2-[4-(7-Methyl-1,8-naphthyridin-4-yl)-1-piperazinyl]ethyl}-2H-1,4-benzoxazin-3(4H)-one (E67)

The title compound (E67) was prepared from 2-methyl-5-(1-piperazinyl)-1,8-naphthyridine (European Journal of Med. Chem. (1999), 34(6), 505-513) according to the general procedure for the alkylation of arylpiperazines with 6-(2-chloroethyl)-4H-benzo[1,4]oxazin-3-one (D4).

### Free Base Analytical Data:

MS; (ES) m/z: 404.5 [MH⁺]. C₂₃H₂₅N₅O₂ requires 433.48.

¹H NMR (500 MHz, DMSO) δ: 8.8 (d, 1H), 8.2 (d, 1H), 7.7 (bs, 1H), 7.2 (bs, 1H), 6.9 (d, 1H), 6.8 (d, 1H), 6.6 (s, 1H), 4.51 (s, 2H), 3.25 (m, 4H), 2.8 (m, 6H), 2.6 (m, 2H).

### Example 68

### 6-{2-[4-(1,6-Naphthyridin-5-yl)-1-piperazinyl]ethyl}-2H-1,4-benzoxazin-3(4H)-one (E68)

The title compound (E68) was prepared from 5-(1-piperazinyl)-1,6-naphthyridine (Bioorganic & Medicinal Chemistry (2001), 9(8), 2129-2137) according to the general procedure for the alkylation of arylpiperazines with 6-(2-chloroethyl)-4H-benzo[1,4]oxazin-3-one (D4).

MS; (ES) m/z: 390.5 [MH⁺]. C₂₃H₂₅N₅O₂ requires 389.46.

¹H NMR (500 MHz, DMSO) δ: 11.79 (bs, 1H), 10.79 (s, 1H), 9.53 (bs, 1H), 8.96 (bs, 1H), 8.6 (bd, 1H), 7.58 (m, 1H), 6.92 (d, 1H), 6.84 (dd, 1H), 6.76 (d, 1H), 6.66 (d, 1H), 4.52 (s, 2H), 3.7-3.4 (bm, 8H), 3.3-3.0 (t/t, 4H).

### Example 69

### 6-{2-[4-(2-Phenylquinolin-5-yl)piperazin-1-yl]ethyl}-4H-benzo[1,4]oxazin-3-one dihydrochloride salt (E69)

The title compound (E69) was prepared in 46% yield according to the general alkylation procedure starting from 2-phenyl-5-piperazin-1-ylquinoline (D56) and 6-(2-chloroethyl)-4H-benzo[1,4]oxazin-3-one (D4).

MS; (ES) m/z: 465.2 [MH]⁺. C₂₉H₂₈N₄O₂ requires 464.

¹H-NMR (500 MHz, DMSO) δ: 10.79 (s, 1H), 10.22 (bs, 1H), 8.58 (d, 1H), 8.26 (d, 2H), 8.13 (d, 1H), 7.82 (d, 1H), 7.72 (t, 1H), 7.56 (t, 2H), 7.50 (t, 1H), 7.26 (d, 1H), 6.94 (d, 1H), 6.85 (dd, 1H), 6.79 (d, 1H), 4.54 (s, 2H), 3.2-3.7 (m, 8H), 3.39 (m, 2H), 3.01 (m, 2H).

### Example 70

### 6-{[4-(7-Fluoro-2-methyl-5-quinolinyl)-1-piperazinyl]acetyl}-2H-1,4-benzoxazin-3(4H)-one (E70)

To a suspension of 7-fluoro-2-methyl-5-piperazin-1-ylquinoline (D30) (1.62 g, 6.60 mmol) and 6-(chloroacetyl)-2H-1,4-benzoxazin-3(4H)-one (1.93 g, 8.52 mmol) in dry acetonitrile (70 ml), N,N-diisopropylethylamine (2.30 ml, 13.21 mmol) was added. The reaction mixture was stirred at reflux for 14 h under nitrogen, then allowed to cool to room temperature and concentrated in vacuo. The residue was dissolved in saturated aqueous NH₄Cl (50 ml) and the mixture extracted with ethyl acetate (2×50 ml). The organic phases were collected, dried (Na₂SO₄) and concentrated in vacuo. The crude product was then triturated with diethyl ether to afford the title compound (E70) as a yellow solid (2.22 g, 77%).

MS; (ES) m/z: 435.20 [MH⁺]. C₂₄H₂₃FN₄O₃ requires 434.47.

¹H-NMR (300 MHz, CDCl₃) δ: 8.28 (d, 1H), 8.11 (bs, 1H), 7.75 (d, 1H), 7.53 (s, 1H), 7.32 (d, 1H), 7.14 (d, 1H), 7.01 (d, 1H), 6.79 (d, 1H), 4.74 (s, 2H), 3.88 (s, 2H), 3.13 (bs, 4H), 2.89 (bs, 4H), 2.66 (s, 3H).

### Example 71

### 6-{2-[4-(7-Fluoro-2-methyl-5-quinolinyl)-1-piperazinyl]-1-hydroxyethyl}-2H-1,4-benzoxazin-3(4H)-one (E71)

A stirred suspension of 6-{[4-(7-fluoro-2-methyl-5-quinolinyl)-1-piperazinyl]acetyl}-2H-1,4-benzoxazin-3(4H)-one (E70). (1.21 g, 2.83 mmol) in dry methanol (10 ml) was cooled to 0° C. and sodium borohydride (0.42 g, 11.1 mmol) added in portions. The reaction mixture was stirred at 0° C. under nitrogen for 4 h, quenched with 10% HCl, passed through a SCX cartridge and then purified by flash chromatography on silica gel, eluting with 2% methanol in DCM to afford the title compound (E71) as a yellow solid (0.75 g, 61%).

MS; (ES) m/z: 437.20 [MH⁺]. C₂₄H₂₅FN₄O₃ requires 436.48.

¹H-NMR (500 MHz, DMSO-d⁶) δ: 11.65 (s, 1H), 8.26 (d, 1H), 7.32 (d, 1H), 7.24 (dd, 1H), 6.96 (dd, 1H), 6.94 (d, 1H), 6.87 (m, 2H), 5.04 (d, 1H), 4.67 (m, 1H), 4.51 (s, 2H), 3.03 (bs, 4H), 2.74 (bs, 4H), 2.60 (s, 3H), 2.54-2.43 (m, 2H).

### Example 72

### 6-{1-Fluoro-2-[4-(7-fluoro-2-methyl-5-quinolinyl)-1-piperazinyl]ethyl}-2H-1,4-benzoxazin-3(4H)-one hydrochloride (E72)

A stirred suspension of 6-{2-[4-(7-fluoro-2-methyl-5-quinolinyl)-1-piperazinyl]-1-hydroxyethyl}-2H-1,4-benzoxazin-3(4H)-one (E71) (38.60 mg, 0.09 mmol) in dry DCM (4 ml) was cooled to 0° C. and DAST (0.02 ml, 0.15 mmol) was added dropwise. The reaction mixture was allowed to warm to room temperature and stirred under nitrogen overnight. Solvent was evaporated and the product crude purified by flash cromatography on silica gel, eluting with 3% methanol in DCM to afford the corresponding free base of the title compound (E72) as a yellow solid (13.3 mg, 34%).

The free base was dissolved in dry methanol (2 ml) and a 1.25 M solution of hydrochloric acid in methanol (0.075 mL, 93.7 mmol) was slowly added at 0° C. The resulting suspension was stirred at 0° C. for 2 h. Evaporation of the volatile gave the title compound (E72) as a yellow solid (12.2 mg, 79%).

MS; (ES) m/z: 439.30 [MH⁺]. C₂₄H₂₄F₂N₄O₂ requires 438.48.

¹H-NMR (500 MHz, DMSO-d⁶) δ: 11.24 (bs, 1H), 10.93 (s, 1H), 8.65 (bd, 1H), 7.62 (d, 1H), 7.57 (d, 1H), 7.29 (d, 1H), 7.06 (m, 2H), 6.99 (s, 1H), 6.25 (dd, 1H), 4.00-3.30 (bs, 10H), 2.79 (bs, 3H).

### Example 73

### 8-Fluoro-6-{2-[4-(2-methyl-5-quinolinyl)-1-piperazinyl]ethyl}-2H-1,4-benzoxazin-3(4H)-one hydrochloride (E73)

The title compound (E73) was prepared in 24% yield according to the general alkylation procedure starting from 2-methyl-5-piperazin-1-ylquinoline (D3) (101 mg, 0.45 mmol) and 6-(2-chloroethyl)-8-fluoro-4H-benzo[1,4]oxazin-3-one (D4) (121 mg, 0.53 mmol).

MS; (ES) m/z: 421.30 [MH⁺]. C₂₄H₂₅FN₄O₂ requires 420.49.

¹H-NMR (500 MHz, DMSO-d⁶) δ: 11.00 (s, 1H), 10.90 (bs, 1H), 8.81 (bs, 1H), 7.88 (bs, 2H), 7.73 (bs, 1H), 7.39 (bs, 1H), 6.81 (dd, 1H), 6.65 (d, 1H), 4.64 (s, 2H), 3.68 (bd, 2H), 3.60-3.40 (bs, 6H), 3.30 (m, 2H), 3.05 (dd, 2H), 2.85 (bs, 3H).

### Example 74

### 8-Fluoro-6-{[4-(2-methyl-5-quinolinyl)-1-piperazinyl]acetyl}-2H-1,4-benzoxazin-3(4H)-one (E74)

The title compound (E74) was prepared in 74% yield in a similar fashion to Example 70 starting from 2-methyl-5-piperazin-1-ylquinoline (D3) (0.39 g, 1.71 mmol) and 6-(chloroacetyl)-8-fluoro-2H-1,4-benzoxazin-3(4H)-one (D59) (0.64 g, 1.89 mmol).

MS; (ES) m/z: 435.20 [MH⁺]. C₂₄H₂₃FN₄O₃ requires 434.47.

¹H-NMR (300 MHz, DMSO-d⁶) δ: 10.97 (s, 1H), 8.18 (d, 1H), 7.53 (d, 1H), 7.45 (m, 2H), 7.29 (s, 1H), 7.25 (d, 1H), 6.97 (d, 1H), 4.63 (s, 2H), 3.72 (s, 2H), 2.90 (bs, 4H), 2.65 (bs, 4H), 2.47 (s, 3H).

### Example 75

### 8-Fluoro-6-{1-hydroxy-2-[4-(2-methyl-5-quinolinyl)-1-piperazinyl]ethyl}-2H-1,4-benzoxazin-3(4H)-one (E75)

The title compound (E75) was prepared in 32% yield in a similar fashion to Example 71 starting from 8-fluoro-6-{[4-(2-methyl-5-quinolinyl)-1-piperazinyl]acetyl}-2H-1,4-benzoxazin-3(4H)-one (E74) (0.26 g, 0.61 mmol).

MS; (ES) m/z: 437.20 [MH⁺]. C₂₄H₂₅FN₄O₃ requires 436.48.

¹H-NMR (500 MHz, DMSO-d⁶) δ: 10.88 (s, 1H), 8.33 (d, 1H), 7.58 (m, 2H), 7.37 (d, 1H), 7.09 (d, 1H), 6.88 (d, 1H), 6.79 (s, 1H), 5.20 (bs, 1H), 4.68 (bs, 1H), 4.63 (s, 2H), 3.02 (bs, 4H), 2.76 (bs, 4H), 2.62 (s, 3H), 2.53 (m, 2H).

### Example 76

### 8-Fluoro-6-{1-fluoro-2-[4-(2-methyl-5-quinolinyl)-1-piperazinyl]ethyl}-2H-1,4-benzoxazin-3(4H)-one hydrochloride (E76)

The title compound (E76) was prepared in 25% yield in a similar fashion to Example 72 starting from 8-fluoro-6-{1-hydroxy-2-[4-(2-methyl-5-quinolinyl)-1-piperazinyl]-ethyl}-2H-1,4-benzoxazin-3(4H)-one (E74) (61.70 mg, 0.14 mmol).

MS; (ES) m/z: 439.30 [MH⁺]. C₂₄H₂₄F₂N₄O₂ requires 438.48.

¹H-NMR (500 MHz, DMSO-d⁶) δ: 11.13 (bs, 1H), 10.78 (bs, 1H), 8.45 (bs, 1H), 7.68 (bs, 1H), 7.48 (bs, 1H), 7.22 (bs, 1H), 6.83 (bs, 1H), 7.06 (bd, 1H), 6.22 (bd, 1H), 4.70 (bs, 2H), 4.00-2.50 (bs, 10H), 2.68 (bs, 3H).

### Example 77

### 8-Fluoro-6-{2-[4-(7-fluoro-2-methyl-5-quinolinyl)-1-piperazinyl]ethyl}-2H-1,4-benzoxazin-3(4H)-one hydrochloride (E77)

The title compound (E77) was prepared in 30% yield according to the general alkylation procedure starting from 7-fluoro-2-methyl-5-piperazin-1-ylquinoline (D30) (61 mg, 0.28 mmol) and 6-(2-chloroethyl)-8-fluoro-4H-benzo[1,4]oxazin-3-one (D4) (79.5 mg, 0.35 mmol).

MS; (ES) m/z: 439.20 [MH⁺]. C₂₄H₂₄F₂N₄O₂ requires 438.48.

¹H-NMR (500 MHz, DMSO-d⁶) δ: 10.99 (s, 1H), 10.98 (bs, 1H), 8.57 (bs, 1H), 7.56 (d, 1H), 7.50 (d, 1H), 7.24 (d, 1H), 6.87 (dd, 1H), 6.62 (s, 1H), 4.63 (s, 2H), 3.70-3.20 (bs, 10H), 3.03 (dd, 2H), 2.74 (s, 3H).

### Example 78

### 8-Fluoro-6-{[4-(7-fluoro-2-methyl-5-quinolinyl)-1-piperazinyl]acetyl}-2H-1,4-benzoxazin-3(4H)-one (E78)

The title compound (E78) was prepared in 74% yield in a similar fashion to Example 70 starting from 7-fluoro-2-methyl-5-piperazin-1-ylquinoline (D30) (0.33 g, 1.36 mmol) and 6-(2-chloroethanoyl)-8-fluoro-4H-benzo[1,4]oxazin-3-one (0.37 g, 1.52 mmol).

MS; (ES) m/z: 453.20 [MH⁺]. C₂₄H₂₂F₂N₄O₃ requires 452.46.

¹H-NMR (300 MHz, DMSO-d⁶) δ: 11.03 (s, 1H), 8.27 (d, 1H), 7.66 (d, 1H), 7.38 (s, 1H), 7.29 (d, 1H), 7.25 (d, 1H), 6.96 (d, 1H), 4.78 (s, 2H), 3.83 (s, 2H), 3.05 (bs, 4H), 2.77 (bs, 4H), 2.59 (s, 3H).

### Example 79

### 8-Fluoro-6-{2-[4-(7-fluoro-2-methyl-5-quinolinyl)-1-piperazinyl]-1-hydroxyethyl}-2H-1,4-benzoxazin-3(4H)-one hydrochloride (E79)

A stirred suspension of (E78) (92.30 mg, 0.20 mmol) in dry methanol (8 ml) was cooled to 0° C. and sodium borohydride (37 mg, 1.02 mmol) was added in portions. The reaction mixture was allowed to warm to room temperature, stirred for 6 h under nitrogen, quenched with 10% HCl, passed through a SCX cartridge and then purified by flash chromatography on silica gel, eluting with 2% methanol in DCM to afford the corresponding free base of the title compound (E79) as a solid (14.8 mg, 16%). The free base was dissolved in dry methanol (3 ml) and a 1.25 M solution of hydrochloric acid in methanol (0.065 ml, 81.2 mmol) was slowly added at 0° C. The resulting suspension was stirred at 0° C. for 2 h. Evaporation of the volatiles gave the title compound (E79) as a solid (13.9 mg, 81%).

MS; (ES) m/z: 455.20 [MH⁺]. C₂₄H₂₄F₂N₄O₃ requires 454.47.

¹H-NMR (500 MHz, DMSO-d⁶) δ: 11.06 (s, 1H), 10.09 (bs, 1H), 8.42 (bd, 1H), 7.47 (d, 1H), 7.42 (d, 1H), 7.17 (d, 1H), 6.96 (d, 1H), 6.85 (s, 1H), 6.42 (bs, 1H), 5.09 (d, 1H), 4.67 (s, 2H), 3.70 (m, 2H), 3.70-3.30 (m, 6H), 2.68 (s, 3H).

### Example 80

### 6-{[4-(8-Chloro-2-methyl-5-quinolinyl)-1-piperazinyl]acetyl}-2H-1,4-benzoxazin-3(4H)-one (E80)

The title compound (E80) was prepared in 46% yield in a similar fashion to Example 70 starting from 8-chloro-2-methyl-5-piperazin-1-ylquinoline (D64) (0.26 g, 1.01 mmol) and 6-(chloroacetyl)-2H-1,4-benzoxazin-3(4H)-one (0.30 g, 1.32 mmol).

MS; (ES) m/z: 451.20 [MH⁺]. C₂₄H₂₃ClN₄O₃ requires 450.92.

¹H-NMR (300 MHz, DMSO-d⁶) δ: 10.91 (s, 1H), 8.37 (d, 1H), 7.76 (d, 1H), 7.69 (d, 1H), 7.60 (s, 1H), 7.48 (d, 1H), 7.12-7.03 (m, 2H), 4.70 (s, 2H), 3.88 (s, 2H), 3.05 (bs, 4H), 2.80 (bs, 4H), 2.71 (s, 3H).

### Example 81

### 6-{2-[4-(8-Chloro-2-methyl-5-quinolinyl)-1-piperazinyl]-1-hydroxyethyl}-2H-1,4-benzoxazin-3(4H)-one hydrochloride (E81)

A stirred suspension of 6-{[4-(8-chloro-2-methyl-5-quinolinyl)-1-piperazinyl]acetyl}-2H-1,4-benzoxazin-3(4H)-one (E80) (0.21 g, 0.47 mmol) in dry methanol (15 ml) was cooled to 0° C. and sodium borohydride (54 mg, 1.41 mmol) was added in portions. The reaction mixture was allowed to warm to room temperature, stirred for 4 h under nitrogen, quenched with 10% HCl, passed through a SCX cartridge and then purified by flash chromatography, eluting with 2% methanol in DCM to afford the corresponding free base of the title compound (E81) as a solid (0.18 g, 84%).

The free base was dissolved in dry methanol (5 ml) and a 1.25 M solution in methanol of hydrochloric acid (0.69 ml, 0.86 mmol) was slowly added at 0° C. The resulting suspension was stirred at 0° C. for 2 h. Evaporation of the volatile gave the title compound (E81) as a solid (0.17 g, 85%).

MS; (ES) m/z: 453.20 [MH⁺]. C₂₄H₂₅ClN₄O₃ requires 452.94.

¹H-NMR (500 MHz, DMSO-d⁶) δ: 10.86 (s, 1H), 10.14 (bs, 1H), 8.44 (d, 1H), 7.83 (d, 1H), 7.54 (d, 1H), 7.18 (d, 1H), 7.00 (m, 3H), 6.31 (bs, 1H), 5.10 (bs, 1H), 4.56 (s, 2H), 3.72-3.22 (bm, 10H), 2.70 (s, 3H).

### Example 82

### 6-{2-[4-(8-Chloro-2-methyl-5-quinolinyl)-1-piperazinyl]ethyl}-2H-1,4-benzoxazin-3(4H)-one hydrochloride (E82)

The title compound (E82) was prepared in 27% yield according to the general alkylation procedure starting from 8-Chloro-2-methyl-5-piperazin-1-ylquinoline (D64) (0.21 g, 0.81 mmol) and 6-(2-chloroethyl)-4H-benzo[1,4]oxazin-3-one (D4) (0.21 g, 0.97 mmol).

MS; (ES) m/z: 437.20 [MH⁺]. C₂₄H₂₅ClN₄O₂ requires 436.94.

¹H-NMR (500 MHz, DMSO-d⁶) δ: 10.75 (s, 1H), 10.44 (bs, 1H), 8.41 (d, 1H), 7.78 (d, 1H), 7.50 (d, 1H), 7.14 (d, 1H), 6.90 (d, 1H), 6.81 (dd, 1H), 6.75 (d, 1H), 4.51 (s, 2H), 3.63-3.40 (bd, 6H), 3.15 (bt, 4H), 2.70 (dd, 2H), 2.67 (s, 3H).

### Example 83

### 4-Methyl-8-{2-[4-(2-methyl-5-quinolinyl)-1-piperazinyl]ethyl}-2H-1,4-benzoxazin-3(4H)-one hydrochloride (E83)

A mixture of (4-methyl-3-oxo-3,4-dihydro-2H-1,4-benzoxazin-8-yl)acetaldehyde (D69) (84 mg, 0.41 mmol) and 2-methyl-5-piperazin-1-yl-quinoline (D30) (0.14 g, 0.61 mmol) in dry 1,2-dichloroethane (5 ml) was stirred at room temperature under nitrogen for 40 min. Sodium triacetoxyborohydride (0.13 g, 0.61 mmol) was then added and the resulting reaction mixture was stirred for 4 h, quenched with a saturated aqueous solution of NaHCO₃ (20 ml) and extracted with dichloromethane (3×20 ml). The organic layers were combined, dried (Na₂SO₄) and concentrated in vacuo. The crude product was purified by SPE cartridge (Si), eluting with 2% methanol in dichloromethane to afford the corresponding free base of the title compound (E83) as a solid (0.11 g, 67%).

The free base was dissolved in dry methanol (3 ml) and a 1.25 M solution of hydrochloric acid in methanol (0.55 ml, 0.69 mmol) was slowly added at 0° C. The resulting suspension was stirred at 0° C. for 4 h. Evaporation of the volatiles gave the title compound (E83) as a solid (0.11 g, 83%).

MS; (ES) m/z: 417.30 [MH⁺]. C₂₅H₂₈N₄O₂ requires 416.52.

¹H-NMR (500 MHz, DMSO-d⁶) δ: 10.78 (bs, 1H), 8.41 (d, 1H), 7.67 (m, 2H), 7.45 (d, 1H), 7.20 (d, 1H), 7.20-7.00 (m, 3H), 4.73 (s, 2H), 3.71 (m, 2H), 3.50-3.00 (m, 10H), 3.30 (s, 3H), 2.67 (s, 3H).

### Example 84

### 8-{2-[4-(2-Methyl-5-quinolinyl)-1-piperazinyl]ethyl}-2H-1,4-benzoxazin-3(4H)-one hydrochloride (E84)

The title compound (E84) was prepared in 42% yield in a similar fashion to Example 83 starting from (3-oxo-3,4-dihydro-2H-1,4-benzoxazin-8-yl)acetaldehyde (D70) (20 mg, 0.10 mmol) and 2-methyl-5-piperazin-1-yl-quinoline (D30) (34 mg, 0.15 mmol).

MS; (ES) m/z: 403.20 [MH⁺]. C₂₄H₂₆N₄O₂ requires 402.50.

¹H-NMR (500 MHz, DMSO-d⁶) δ: 10.76 (s, 1H), 10.19 (bs, 1H), 8.41 (d, 1H), 7.67 (m, 2H), 7.45 (d, 1H), 7.21 (bd, 1H), 6.98-6.85 (m, 3H), 4.65 (s, 2H), 3.80-3.00 (bm, 12H), 2.70 (s, 3H).

### Example 85

### 6-{2-[4-(7-Chloro-2-methyl-5-quinolinyl)-1-piperazinyl]ethyl}-7-fluoro-2H-1,4-benzoxazin-3(4H)-one hydrochloride (E85)

The title compound (E85) was prepared in 32% yield according to the general alkylation procedure starting from 7-chloro-2-methyl-5-piperazin-1-ylquinoline (D9) (51 mg, 0.20 mmol) and 6-(2-chloroethyl)-7-fluoro-4H-benzo[1,4]oxazin-3-one (D24) (63 mg, 0.27 mmol).

MS; (ES) m/z: 455.30 [MH⁺]. C₂₄H₂₄ClFN₄O₂ requires 454.93.

¹H-NMR (500 MHz, DMSO-d⁶) δ: 11.40 (bs, 1H), 10.86 (s, 1H), 8.73 (s, 1H), 7.96 (d, 1H), 7.72 (d, 1H), 7.04 (d, 1H), 6.95 (d, 1H), 6.83 (d, 1H), 4.58 (s, 2H), 3.71-3.52 (bm, 4H), 3.52-3.38 (bm, 6H), 3.32 (bm, 2H), 3.09 (bm, 2H), 2.84 (s, 3H).

### Example 86

### 6-{2-[(2S)-2-Methyl-4-(2-methyl-5-quinolinyl)-1-piperazinyl]ethyl}-2H-1,4-benzoxazin-3(4H)-one hydrochloride (E86)

The title compound (E86) was prepared in 32% yield according to the general alkylation procedure starting from 2-methyl-5-[(3S)-3-methyl-1-piperazinyl]quinoline (D72) (48 mg, 0.20 mmol) and 6-(2-chloroethyl)-4H-benzo[1,4]oxazin-3-one (D4) (55 mg, 0.26 mmol).

MS; (ES) m/z: 417.30 [MH⁺]. C₂₅H₂₈N₄O₂ requires 416.52.

¹H-NMR (500 MHz, DMSO-d⁶) δ: 11.10 (bs, 1H), 10.80 (s, 1H), 8.71 (bs, 1H), 7.83 (bs, 2H), 7.65 (bs, 1H), 7.34 (bs, 1H), 7.00-6.80 (m, 3H), 4.57 (s, 2H), 3.95 (m, 1H), 3.80-3.00 (m, 10H), 2.80 (s, 3H), 1.58 (d, 3H).

### Example 87

### 6-{2-[(2R)-2-Methyl-4-(2-methyl-5-quinolinyl)-1-piperazinyl]ethyl}-2H-1,4-benzoxazin-3(4H)-one hydrochloride (E87)

The title compound (E87) was prepared in 18% yield according to the general alkylation procedure starting from 2-methyl-5-[(3R)-3-methyl-1-piperazinyl]quinoline (D71) (44 mg, 0.18 mmol) and 6-(2-chloroethyl)-4H-benzo[1,4]oxazin-3-one (D4) (50 mg, 0.24 mmol).

MS; (ES) m/z: 417.30 [MH⁺]. C₂₅H₂₈N₄O₂ requires 416.52.

¹H-NMR (500 MHz, DMSO-d⁶) δ: 10.94 (bs, 1H), 10.80 (s, 1H), 8.71 (bs, 1H), 7.83 (bs, 2H), 7.65 (bs, 1H), 7.34 (bs, 1H), 7.00-6.80 (m, 3H), 4.57 (s, 2H), 3.78 (m, 1H), 3.80-3.00 (m, 10H), 2.80 (s, 3H), 1.45 (d, 3H).

### Examples E88-E102

The title compounds (E88-E102) were prepared according to the general alkylation procedure starting from the appropriate arylpiperazine and 6-(2-chloroethyl)-4H-benzo[1,4]oxazin-3-one (D4) or 6-(2-chloropropyl)-4H-benzo[1,4]oxazin-3-one.

| Example | NMR | [MH]⁺ |
|---|---|---|
| 6-{2-[4-(2,3-dihydro-1,4- | 1H-NMR (500 MHz, DMSO) | MS; (ES) m/z: |
| benzodioxin-6-yl)-1- | δ: 10.78 (s, 1H), 10.58 (bs, 1H), | 396 [MH⁺]. C22H25N3O4 |
| piperazinyl]ethyl}-2H-1,4- | 6.92 (d, 1H), 6.82 (dd, 1H), | requires 395. |
| benzoxazin-3(4H)-one | 6.76 (m, 2H), 6.50 (m, 2H), | |
| hydrochloride | 4.53 (s, 2H), 4.20 (m, 2H), | |
| (E88) | 4.15 (m, 2H), 3.65 (d, 2H), | |
| | 3.58 (d, 2H), 3.27 (m, 2H), | |
| | 3.14 (m, 2H), 2.98 (m, 4H). | |
| 6-{2-[4-(3,4-dihydro-2H- | 1H-NMR (500 MHz, DMSO) | MS; (ES) m/z: |
| 1,5-benzodioxepin-7-yl)-1- | δ: 10.78 (s, 1H), 10.56 (bs, 1H), | 410 [MH⁺]. C23H27N3O4 |
| piperazinyl]ethyl}-2H-1,4- | 6.92 (d, 1H), 6.87 (d, 1H), | requires 409. |
| benzoxazin-3(4H)-one | 6.81 (dd, 1H), 6.76 (d, 1H), | |
| hydrochloride | 6.62 (d, 1H), 6.58 (dd, 1H), | |
| (E89) | 4.54 (s, 2H), 4.08 (m, 2H), | |
| | 4.01 (m, 2H), 3.71 (d, 2H), | |
| | 3.58 (d, 2H), 3.28 (m, 2H), | |
| | 3.13 (m, 2H), 3.00 (m, 2H), | |
| | 2.97 (m, 2H), 2.05 (m, 2H). | |
| 6-{2-[4-(7-bromo-1H-indol- | 1H-NMR (500 MHz, DMSO) | MS; (ES) m/z: |
| 4-yl)-1-piperazinyl]ethyl}- | δ: 11.2 (bs, 1H), 10.6 (bs, 1H), | 456 [MH⁺]. |
| 2H-1,4-benzoxazin-3(4H)- | 7.28 (t, 1H), 7.15 (d, 1H), | C22H23BrN4O2 |
| one | 6.85 (d, 1H), 6.79 (dd, 1H), | requires 455. |
| (E90) | 6.77 (d, 1H), 6.50 (m, 1H), | |
| | 6.40 (d, 1H), 4.51 (s, 2H), | |
| | 3.11-2.65 (bm-bm, 4H-4H), | |
| | 2.68 (m, 2H), 2.51 (m, 2H). | |
| 6-{3-[4-(7-bromo-1H-indol- | 1H-NMR (500 MHz, DMSO) | MS; (ES) m/z: |
| 4-yl)-1- | δ: 11.18 (bs, 1H), | 470 [MH⁺]. |
| piperazinyl]propyl}-2H- | 10.62 (bs, 1H), 7.28 (t, 1H), | C23H25BrN4O2 |
| 1,4-benzoxazin-3(4H)-one | 7.14 (d, 1H), 6.84 (d, 1H), | requires 469. |
| (E91) | 6.76 (dd, 1H), 6.73 (d, 1H), | |
| | 6.49 (m, 1H), 6.40 (d, 1H), | |
| | 4.51 (s, 2H), 3.11-2.57 (bm-bm, 4H- | |
| | 4H), 2.53 (t, 2H), 2.34 (t, 2H), | |
| | 1.71 (q, 2H). | |
| 6-{2-[4-(1-isoquinolinyl)-1- | 1H-NMR (500 MHz, DMSO) | MS; (ES) m/z: |
| piperazinyl]ethyl}-2H-1,4- | δ: 10.64 (s, 1H), 8.10 (m, 2H), | 389 [MH⁺]. C23H24N4O2 |
| benzoxazin-3(4H)-one | 7.88 (d, 1H), 7.68 (t, 1H), | requires 390. |
| (E92) | 7.60 (t, 1H), 7.37 (d, 1H), | |
| | 6.86-6.78 (m, 3H), | |
| | 4.51 (s, 2H), 2.7 (m, 2H), | |
| | 2.66-2.56 (m, 4H), 2.5 (m, 6H). | |
| ethyl 5-{4-[2-(3-oxo-3,4- | 1H-NMR (500 MHz, CD₃OD) | MS; (ES) m/z: |
| dihydro-2H-1,4- | δ: 7.53 (d, 1H), 7.48 (d, 1H), | 450 [MH⁺]. C25H27N3O5 |
| benzoxazin-6-yl)ethyl]-1- | 7.27 (dd, 1H), 7.25 (d, 1H), | requires 449. |
| piperazinyl}-1- | 6.9-6.84 (m, 2H), 6.79 (d, 1H), | |
| benzofuran-2-carboxylate | 4.53 (s, 2H), 4.10 (q, 2H), | |
| (E93) | 3.23 (m, 4H), | |
| | 2.78-2.66 (m, 4H), 2.75 (m, 4H), | |
| | 1.24 (t, 3H). | |
| 6-{2-[4-(1,2-dihydro-5- | 1H-NMR (500 MHz, DMSO) | MS; (ES) m/z: |
| acenaphthylenyl)-1- | δ: 10.63 (s, 1H), 7.64 (d, 1H), | 414 [MH⁺]. C26H27N3O2 |
| piperazinyl]ethyl}-2H-1,4- | 7.42 (t, 1H), 7.28 (d, 1H), | requires 413. |
| benzoxazin-3(4H)-one | 7.19 (d, 1H), 6.98 (d, 1H), | |
| (E94) | 6.87 (d, 1H), 6.79 (d, 1H), | |
| | 6.78 (d, 1H), 4.51 (s, 2H), | |
| | 3.33-3.0 (m, 4H), | |
| | 3.0-2.7 (m, 8H), 2.7 (m, 2H), | |
| | 2.58 (m, 2H). | |
| 6-{2-[4-(5-fluoro-1H-indol- | 1H-NMR (300 MHz, CD₃OD) | MS; (ES) m/z: |
| 3-yl)-1-piperidinyl]ethyl}- | δ: 7.19 (m, 2H), 7.02 (s, 1H), | 394 [MH⁺]. C23H24FN3O2 |
| 2H-1,4-benzoxazin-3(4H)- | 6.7/6.8 (m, 4H), 4.45 (s, | requires 393. |
| one (E95) | 2H), 3.42 (d, 2H), 2.73/ | |
| | 3.02 (m, 7H), 2.14 (d, 2H), | |
| | 1.87 (m, 2H). | |
| 6-{2-[4-(5-chloro-1H-indol- | 1H-NMR (500 MHz, DMSO) | MS; (ES) m/z: |
| 4-yl)-1-piperazinyl]ethyl}- | δ: 11.21 (bs, 1H), 10.63 (bs, 1H), | 411 [MH⁺]. |
| 2H-1,4-benzoxazin-3(4H)- | 7.31 (t, 1H), 7.10 (d, 1H), | C22H23ClN4O2 |
| one (E96) | 7.03 (d, 1H), 6.85 (d, 1H), | requires 410. |
| | 6.78 (m, 2H), 6.62 (s, 1H), | |
| | 4.51 (s, 2H), 3.25 (m, 4H), | |
| | 2.68 (m, 2H), 2.60 (m, 4H), | |
| | 2.51 (m, 2H). | |
| 6-{2-[4-(6-chloro-1H-indol- | 1H-NMR (500 MHz, DMSO) | MS; (ES) m/z: |
| 4-yl)-1-piperazinyl]ethyl}- | δ: 11.13 (bs, 1H), 10.63 (bs, | 411 [MH⁺]. |
| 2H-1,4-benzoxazin-3(4H)- | 1H), 7.25 (t, 1H), 7.02 (s, 1H), | C22H23ClN4O2 |
| one (E97) | 6.85 (d, 1H), 6.77 (m, 2H), | requires 410. |
| | 4.51 (s, 2H), 3.14 (m, 4H), | |
| | 2.68 (m, 2H), 2.65 (m, 4H), | |
| | 2.53 (m, 2H). | |
| 6-{2-[4-(1H-pyrrolo[2,3- | 1H-NMR (500 MHz, DMSO) | MS; (ES) m/z: |
| b]pyridin-4-yl)-1- | δ: 11.38 (bs, 1H), 10.64 (bs, | 378 [MH⁺]. C21H23N5O2 |
| piperazinyl]ethyl}-2H-1,4- | 1H), 7.94 (d, 1H), 7.22 (dd, | requires 377. |
| benzoxazin-3(4H)-one | 1H), 6.84 (d, 1H), 6.79 (dd, 1H), | |
| (E98) | 6.77 (d, 1H), 6.44 (dd, 1H), | |
| | 6.40 (d, 1H), 4.51 (s, 2H), | |
| | 3.38 (m, 4H), 2.69 (t, 2H), | |
| | 2.62 (m 4H), 2.5 (t, 2H). | |
| 6-{2-[4-(7-chloro-1H-indol- | 1H-NMR (500 MHz, DMSO) | MS; (ES) m/z: |
| 4-yl)-1-piperazinyl]ethyl}- | δ: 11.3 (bs, 1H), 10.63 (bs, 1H), | 411 [MH⁺]. |
| 2H-1,4-benzoxazin-3(4H)- | 7.31 (t, 1H), 7.01 (d, 1H), | C22H23ClN4O2 |
| one (E99) | 6.84 (d, 1H), 6.78 (dd, 1H), | requires 410. |
| | 6.77 (d, 1H), 6.46 (t, 1H), | |
| | 6.43 (d, 1H), 4.51 (s, 2H), | |
| | 3.11 (m, 4H), 2.65 (m, 6H), | |
| | 2.51 (m, 2H). | |
| 6-{3-[4-(1H-pyrrolo[2,3- | 1H-NMR (500 MHz, DMSO) | MS; (ES) m/z: |
| b]pyridin-4-yl)-1- | δ: 11.37 (bs, 1H), 10.62 (bs, | 392 [MH⁺]. C22H25N5O2 |
| piperazinyl]propyl}-2H- | 1H), 7.93 (d, 1H), 7.21 (dd, | requires 391. |
| 1,4-benzoxazin-3(4H)-one | 1H), 6.84 (d, 1H), 6.76 (dd, | |
| (E100) | 1H), 6.73 (d, 1H), 6.43 (dd, | |
| | 1H), 6.39 (d, 1H), 4.51 (s, 2H), | |
| | 3.37 (m, 4H), 2.53 (m, 6H), | |
| | 2.33 (t, 2H), 1.71 (q, 2H). | |
| 6-{3-[4-(5-chloro-1H-indol- | 1H-NMR (500 MHz, DMSO) | MS; (ES) m/z: |
| 4-yl)-1- | δ: 11.22 (bt, 1H), 10.62 (bs, | 425 [MH⁺]. |
| piperazinyl]propyl}-2H- | 1H), 7.31 (t, 1H), 7.10 (d, 1H), | C23H25ClN4O2 |
| 1,4-benzoxazin-3(4H)-one | 7.03 (d, 1H), 6.84 (d, 1H), | requires 424. |
| (E101) | 6.77 (dd, 1H), 6.74 (d, | |
| | 1H), 6.63 (t, 1H), 4.51 (s, 2H), | |
| | 3.30 (m, 2H), 3.26 (m, 4H), | |
| | 2.5 (m, 4H), 2.35 (m, 2H), | |
| | 1.71 (q, 2H). | |
| 6-{2-[4-(5- | 1H-NMR (500 MHz, DMSO) | MS; (ES) m/z: |
| methylthieno[2,3- | δ: 11.12 (bs, 1H), 10.79 (s, 1H), | 410 [MH⁺]. C21H23N5O2S |
| d]pyrimidin-4-yl)-1- | 8.63 (s, 1H), 7.48 (s, 1H), | requires 409. |
| piperazinyl]ethyl}-2H-1,4- | 6.92 (d, 1H), 6.83 (dd, 1H), | |
| benzoxazin-3(4H)-one | 6.78 (d, 1H), 4.54 (s, 2H), | |
| hydrochloride | 3.93 (d, 2H), 3.62 (d, 2H), | |
| (E102) | 3.49 (m, 2H), 3.30 (m, 4H), | |
| | 3.01 (m, 2H), 2.55 (s, 3H). | |

### Example 103

### 6-({2-[4-(2-methyl-5-quinazolinyl)-1-piperazinyl]ethyl}oxy)-2H-1,4-benzoxazin-3(4H)-one hydrochloride (E103)

The title compound (E103) was prepared from 2-methyl-5-piperazin-1-ylquinazoline (D12) and 6-(2-bromoethoxy)-4H-benzo[1,4]oxazin-3-one (D36) according to the general procedure described for Example 1. Yield 68%.

MS; (ES) m/z: 420 [MH⁺]. C₂₄H₂₆N₄O₃ requires 419.

¹H-NMR (300 MHz, CDCl₃) δ: 9.50 (br s, 1H), 8.42 (s, 1H), 7.7 (t, 1H), 7.55 (d, 1H), 7.05 (d, 1H), 6.8 (d, 1H), 6.5 (m, 1H), 6.4 (s, 1H), 4.55 (s, 2H), 4.05 (t, 2H), 3.2 (m, 4H), 2.85 (m, 10H).

### Example 104

### 6-{2-[4-(7-Chloro-2-methylquinolin-5-yl)piperazin-1-yl]ethanoyl}-4H-benzo[1.4]oxazin-3-one hydrochloride salt (E104)

7-Chloro-2-methyl-5-piperazin-1-yl-quinoline (D9) (90 mg, 0.34 mmol, 1.0 eq.) and 6-(2-chloroethanoyl)-4H-benzo[1,4]oxazin-3-one (100 mg, 0.44 mmol, 1.3 eq.) were added to a solution of N,N-diisopropylethylamine (1.1 ml) in dry acetonitrile (3 ml). The reaction mixture was stirred at reflux for 5 h, then allowed to cool to room temperature and concentrated in vacuo. The residue was dissolved in water (15 ml) and extracted with ethyl acetate (3×15 ml). The organic phases were combined, dried (Na₂SO₄) and concentrated in vacuo. The crude product was passed through a SCX cartridge then purified by flash chromatography, eluting with 3% methanol in DCM affording the title compound (E104) (87 mg, yield 57%).

MS; (ES) m/z: 451 [MH⁺]. C₂₄H₂₃ClN₄O₃ requires 450.

¹H-NMR (300 MHz, DMSO) δ: 11.03 (s, 1H), 10.8 (bs, 1H), 8.3 (d, 1H), 7.7 (d, 1H), 7.6 (m, 2H), 7.4 (d, 1H), 7.05 (m, 2H), 4.68 (s, 2H), 3.1 (bs, 4H), 2.8 (bs, 4H), 2.6 (bs, 3H).

### Example 105

### 6-{2-[4-(7-Chloro-2-methyl-5-quinolinyl)-1-piperazinyl]-1-hydroxyethyl}-2H-1,4-benzoxazin-3(4H)-one (E105)

A stirred suspension of 6-{2-[4-(7-chloro-2-methylquinolin-5-yl)piperazin-1-yl]ethanoyl}-4H-benzo[1.4]oxazin-3-one (E104) (85 mg, 0.19 mmol) in dry methanol (4 ml) was cooled to 0° C. and sodium borohydride (29 mg, 0.76 mmol, 4.0 eq) was added portionwise. The reaction mixture was stirred at room temperature under nitrogen for 4 h. The reaction was quenched with a saturated aq. solution of ammonium chloride (15 ml) and extracted into ethyl acetate (3×20 ml). The organic layers were combined, dried (Na₂SO₄) and concentrated in vacuo. The crude product was purified by flash chromatography, eluting with 5% methanol in DCM affording the title compound (E105) (55 mg, yield 64%).

MS; (ES) m/z: 453 [MH⁺]. C₂₄H₂₅ClN₄O₃ requires 452.

¹H-NMR (500 MHz, DMSO) δ: 10.67 (s, 1H), 8.29 (d, 1H), 7.59 (d, 1H), 7.40 (d, 1H), 7.03 (d, 1H), 6.95 (d, 1H), 6.89 (m, 2H), 5.05 (d, 1H), 4.68 (m, 1H), 4.52 (s, 2H), 3.04 (m, 4H), 2.75 (m, 4H), 2.5 (m, 2H), 2.62 (s, 3H).

### Example 106

### 6-{2-[4-(7-Chloro-2-methyl-5-quinolinyl)-1-piperazinyl]-1-fluoroethyl}-2H-1,4-benzoxazin-3(4H)-one (E106)

A stirred suspension of 6-{2-[4-(7-chloro-2-methyl-5-quinolinyl)-1-piperazinyl]-1-hydroxyethyl}-2H-1,4-benzoxazin-3(4H)-one (E105) (40 mg, 0.09 mmol, 1.0 eq.) in dry DCM (2 ml) was cooled to 0° and DAST (30 uL, 0.23 mmol, 2.5 eq) was added dropwise. The reaction mixture was stirred at 0° C. under nitrogen for 1 h and at r.t. overnight. The reaction was quenched with a saturated aq. solution of sodium carbonate (10 ml) and extracted into DCM (3×10 ml). The organic layers were combined, dried (Na₂SO₄) and concentrated in vacuo. The crude product was purified by flash chromatography, eluting with 2% methanol in DCM to give the title compound (E106) (12 mg, 29%).

MS; (ES) m/z: 455 [MH⁺]. C₂₄H₂₄ClFN₄O₂ requires 454.

¹H-NMR (500 MHz, DMSO) δ: 10.76 (s, 1H), 8.29 (d, 1H), 7.60 (d, 1H), 7.40 (d, 1H), 7.05 (d, 1H), 6.96 (m, 3H), 5.8/5.65 (m, 1H), 4.47 (s, 2H), 3.06 (m, 4H), 2.81 (m, 4H), 3.00/2.7 (m, 2H), 2.62 (s, 3H).

### Example 107

### 6-{3-[4-(2,2-Dimethyl-2,3-dihydro-1-benzofuran-7-yl)-1-piperazinyl]propyl}-2H-1,4-benzoxazin-3(4H)-one (E107)

The title compound (E107) was prepared in 46% yield according to the general alkylation procedure starting from 2,2-dimethyl-2,3-dihydro-1-benzofuran-7-yl)piperazine and 6-(2-chloropropyl)-4H-benzo[1,4]oxazin-3-one.

MS; (ES) m/z: 422 [MH]⁺. C₂₅H₃₁N₃O₃ requires 421.

¹H-NMR (500 MHz, DMSO) δ: 10.6 (bs, 1H), 6.83 (d, 1H), 6.79/6.73 (m, 3H), 6.69 (t, 1H), 6.61 (d, 1H), 4.50 (s, 2H), 3.12 (m, 4H), 2.83 (s, 2H), 2.5 (m, 6H), 2.28 (t, 2H), 1.67 (m, 2H), 1.39 (s, 6H).

### Example 108

### 6-{2-[4-(2,2-dimethyl-2,3-dihydro-1-benzofuran-7-yl)-1-piperazinyl]ethyl}-2H-1,4-benzoxazin-3(4H)-one (E108)

The title compound (E108) was prepared in 46% yield according to the general alkylation procedure starting from 2,2-dimethyl-2,3-dihydro-1-benzofuran-7-ylpiperazine (D75) and 6-(2-chloroethyl)-4H-benzo[1,4]oxazin-3-one (D4).

MS; (ES) m/z: 408 [MH]⁺. C₂₄H₂₉N₃O₃ requires 407.

¹H-NMR (500 MHz, DMSO) δ: 10.62 (bs, 1H), 6.83 (d, 1H), 6.79/6.73 (m, 3H), 6.69 (t, 1H), 6.63 (d, 1H), 4.50 (s, 2H), 3.18 (m, 4H), 2.94 (s, 2H), 2.5 (m, 6H), 1.39 (s, 6H).

### Example 109

### 4-Methyl-6-{[4-(1H-pyrrolo[2,3-b]pyridin-3-yl)-3,6-dihydro-1(2H)-pyridinyl]acetyl}-2H-1,4-benzoxazin-3(4H)-one (E109)

3-(1,2,3,6-Tetrahydro-4-pyridinyl)-1H-pyrrolo[2,3-b]pyridine (Bioorg. Med. Chem. Lett. 2002, 12(3), 307-310) (50 mg, 0.25 mmol) and 6-(chloroacetyl)-4-methyl-2H-1,4-benzoxazin-3(4H)-one (D94) (60 mg, 0.26 mmol) were added to a suspension of potassium carbonate (60 mg, 0.44 mmol) in dry THF (5 ml). The reaction mixture was stirred at reflux for 2 h, then allowed to cool to r.t., filtered and concentrated in vacuo. The residue was triturated with hot methanol to give the title compound (E109) as an orange solid (45 mg, yield 45%).

MS; (ES) m/z: 403 [MH⁺]. C₂₃H₂₂N₄O₃ requires 402.

¹H-NMR (500 MHz, d⁶-DMSO) δ(ppm): 11.69 (bs, 1H), 8.27 (d, 2H), 7.86 (dd, 1H), 7.80 (d, 1H), 7.57 (d, 1H), 7.14 (m, 2H), 6.22 (bt, 1H), 4.83 (bs, 2H), 4.00 (s, 2H), 3.40 (s, 3H), 3.32 (bm, 2H), 2.87 (m, 2H), 2.58 (bm, 2H).

### Example 110

### 6-{1-hydroxy-2-[4-(1H-pyrrolo[2,3-b]pyridin-3-yl)-3,6-dihydro-1(2H)-pyridinyl]ethyl}-4-methyl-2H-1,4-benzoxazin-3(4H)-one (E110)

Sodium borohydride (20 mg, 0.50 mmol) was added to a stirred suspension of 4-methyl-6-{[4-(1H-pyrrolo[2,3-b]pyridin-3-yl)-3,6-dihydro-1(2H)-pyridinyl]acetyl}-2H-1,4-benzoxazin-3(4H)-one (E109) (40 mg, 0.10 mmol) in dry MeOH (5 ml). The reaction mixture was stirred at room temperature under nitrogen for 3 h, then quenched with brine (7 ml), diluted with water (3 ml) and extracted into DCM (3×10 ml). The organic layers were combined, dried (Na₂SO₄) and concentrated in vacuo. The crude product was purified by flash chromatography, eluting with 2-5% methanol in DCM to give the title compound (E110) as a colourless solid (20 mg, yield 50%).

MS; (ES) m/z: 405 [MH⁺]. C₂₃H₂₄N₄O₃ requires 404.

¹H-NMR (500 MHz, d⁶-DMSO) δ(ppm): 11.60 (bs, 1H), 8.18 (d, 2H), 7.47 (d, 1H), 7.14 (d, 1H), 7.06 (m, 1H), 6.99 (dd, 1H), 6.92 (d, 1H), 6.13 (bt, 1H), 5.05 (d, 1H), 4.75 (m, 1H), 4.59 (s, 2H), 3.26 (s, 3H), 3.21 (bm, 1H), 2.75-2.50 (m, 7H).

### Example 111

### 6-{[4-(2-Methyl-5-quinolinyl)-1-piperazinyl]methyl}-2H-1,4-benzoxazin-3(4H)-one (E111)

To a stirred solution of 2-methyl-5-(1-piperazinyl)quinoline (80 mg, 0.35 mmol) (d3) in dry 1,2-dichloroethane (2 ml) at r.t., 3-oxo-3,4-dihydro-2H-1,4-benzoxazine-6-carbaldehyde (WO2002/034754) (81 mg, 0.46 mmol) was added. The solution was left under stirring at r.t. for 1 h then glacial acetic acid (0.028 ml, 0.46 mmol) and sodium triacetoxyborohydride (134 mg, 0.64 mmol) were added sequentially. The reaction was left under stirring for 18 h then it was washed with saturated aqueous solution of NaHCO₃, brine and dried (Na₂SO₄). The solvent was removed under reduced pressure and the resulting crude was purified by flash chromatography (silica, DCM/MeOH 95/5 as eluant).

The title compound (E111) was obtained as a white solid (89 mg, yield=65%).

MS: (ES/+) m/z: 389 [MH⁺]. C₂₃H₂₄N₄O₂ requires 388.

¹H-NMR (300 MHz, d⁶-DMSO) δ(ppm): 10.7 (bs, 1H), 8.3 (d, 1H), 7.6-7.5 (m, 2H), 7.35 (d, 1H), 7.1 (dd, 1H), 6.95-6.85 (m, 3H), 4.5 (s, 2H), 3.5 (bs, 2H), 3.3 (bs, 4H) 3.0 (bs, 4H), 2.6 (s, 3H).

### Example 112

### 4-methyl-6-{[4-(2-methyl-5-quinolinyl)-1-piperazinyl]acetyl}-3,4-dihydro-2H-1,4-benzoxazin-2-one (E112)

To a stirred solution of 2-methyl-5-(1-piperazinyl)quinoline (D3) (1 g, 44 mmol) in dry THF (10 ml), 6-(chloroacetyl)-4-methyl-2H-1,4-benzoxazin-3(4H)-one (D94) and K₂CO₃ (1.22 g, 88 mmol) were added and the mixture was heated at reflux for 4 h. The reaction mixture was then filtered and the solvent removed in vacuo. The residue was diluted in DCM, washed with water, brine and dried (Na₂SO₄). Evaporation of the solvent under reduced pressure afforded a crude foam which was purified by flash chromatography (silica, DCM/MeOH 97/3 as eluant). The title compound (E112) was obtained as a yellow solid (1.1 g, 60%).

MS: (ES/+) m/z: 431 [MH⁺]. C₂₅H₂₆N₄O₃ requires 430.

¹H-NMR (300 MHz, CDCl₃) δ(ppm): 8.3 (d, 1H), 7.7 (m, 2H), 7.6 (d, 1H), 7.2 (d, 2H), 7.0 (d, 2H), 4.7 (s, 2H), 3.85 (s, 2H), 3.3 (s, 3H) 3.2 (bs, 4H), 2.9 (bs, 4H), 2.6 (s, 3H).

### Example 113

### 4-Methyl-6-(1-{[4-(2-methyl-5-quinolinyl)-1-piperazinyl]methyl}ethenyl)-3,4-dihydro-2H-1,4-benzoxazin-2-one (E113)

To a stirred suspension of triphenylphosphonium bromide (0.373 g, 1.04 mmol) in dry THF (15 ml) under inert atmosphere at 0° C., butyl lithium (1.6M solution in hexane, 0.52 ml, 0.84 mmol) was added dropwise. The solution was left under stirring at 0° C. for ˜30 min then a solution of 4-methyl-6-{[4-(2-methyl-5-quinolinyl)-1-piperazinyl]acetyl}-3,4-dihydro-2H-1,4-benzoxazin-2-one (E112) (0.30 g, 0.70 mmol) in dry tetrahydrofuran (5 ml) was added dropwise. The reaction was allowed to return to r.t. and left under stirring for 4 h. As monitoring of the reaction showed that starting material was still present, in a different reaction vessel triphenylphosphonium bromide (0.25 g, 0.69 mmol) dissolved in dry THF (10 ml) at 0° C. was treated with butyl lithium (1.6M solution in hexane, 0.35 ml, 0.56 mmol) as described above. After 30 min the resulting solution was added to the previous one cooled at 0° C. and the reaction mixture was then left under stirring for 16 h. The reaction was then carefully quenched by addition of water (2 ml) and the organic solvent was concentrated in vacuo. The resulting solution was diluted with DCM and washed with water, brine and dried (Na₂SO₄). Removal of the solvent under reduced pressure afforded a crude oil which was purified by chromatography (silica cartridge, DCM/MeOH 99/1 as eluant). The title compound (E113) was obtained as a pale yellow foam (50 mg, 17%).

MS: (ES/+) m/z: 429 [MH⁺]. C₂₆H₂₈N₄O₂ requires 428.

¹H-NMR (300 MHz, CDCl₃) δ(ppm): 8.35 (d, 1H), 7.75-7.45 (m, 4H), 7.3 (s, 1H), 7.05 (d, 2H), 6.95 (d, 2H), 5.5 (br s 1H), 5.25 (bs, 1H) 4.55 (s, 2H), 3.4 (bs, 2H), 3.15 (bs, 2H), 2.7 (bs, 7H).

### Example E114

### 6-(2-Hydroxy-1-{[4-(2-methyl-5-quinolinyl)-1-piperazinyl]methyl}ethyl)-4-methyl-3,4-dihydro-2H-1,4-benzoxazin-2-one hydrochloride (E114)

To a solution of 4-methyl-6-(1-{[4-(2-methyl-5-quinolinyl)-1-piperazinyl]methyl}ethenyl)-3,4-dihydro-2H-1,4-benzoxazin-2-one (E113) (50 mg, 0.1116 mmol) in dry THF (3 ml) at 0° C. under inert atmosphere, BH₃.THF complex (1.0M solution in THF, 0.75 ml, 0.75 mmol) was added dropwise. The reaction was allowed to return to r.t. then water (1 ml) was added dropwise. THF (3 ml) was added to avoid precipitation then a 2.5N solution of NaOH (1 ml) and H₂O₂ (36% solution in water, 0.2 ml) were added sequentially. The reaction solution was left under stirring for 5 h, then it was diluted with DCM and the organic layer was washed with 5% solution of Na₂S₂O₃, water, brine and dried (Na₂SO₄). Removal of the solvent afforded a crude product which was purified by chromatography (silica cartridge, DCM/MeOH 98.5/1.5 then 95/5). The free base of the title compound was obtained as a pale yellow solid (7 mg, 13% yield). The free base (7 mg, 0.016 mmol) was dissolved in DCM (1 ml) and a 1M solution of HCl in Et₂O (0.033 ml, 0.034 mmol) was added at r.t. and the solution was left under stirring for 10 min. The solvent was then evaporated in vacuo and the crude was triturated with Et₂O. Evaporation of the solvent and drying afforded the title compound (E114) as a yellow solid (7.8 mg).

MS: (ES/+) m/z: 447 [MH⁺]. C₂₆H₃₀N₄O₃ requires 446.

¹H-NMR (400 MHz, d⁶-DMSO) δ(ppm): 9.1 (d, 1H), 8.0 (t, 1H), 7.9 (m, 2H), 7.6 (d, 1H), 7.2 (d, 1H) 7.1 (dd, 1H) 7.0 (d, 1H), 4.6 (s, 2H), 3.9-3.3 (m, 13H), 3.4 (s, 3H), 3.0 (s, 3H).

### Example E115

### 6-{[4-(6-fluoro-2-methyl-5-quinolinyl)-1-piperazinyl]acetyl}-2H-1,4-benzoxazin-3(4H)-one (E115)

The title compound (E115) was prepared in 40% yield according to a similar procedure to E104 starting from 6-fluoro-2-methyl-5-(1-piperazinyl)quinoline (D100) and commercially available 6-(chloroacetyl)-2H-1,4-benzoxazin-3(4H)-one.

MS: (ES/+) m/z: 435 [MH⁺]. C₂₄H₂₃FN₄O₃ requires 434.

¹H-NMR (300 MHz, CDCl₃) δ(ppm): 8.75 (s, 1H), 8.55 (d, 1H), 7.75 (m, 2H), 7.6 (s, 1H), 7.35 (d, 1H) 7.3 (d, 1H) 7.0 (d, 1H), 4.7 (s, 2H), 3.8 (s, 2H), 3.5 (bs, 2H), 3.0 (bs, 4H), 2.7 (s, 3H), 2.6 (bs, 2H).

### Example E116

### 6-{2-[4-(6-Fluoro-2-methyl-5-quinolinyl)-1-piperazinyl]-1-hydroxyethyl}-2H-1,4-benzoxazin-3(4H)-one (E116)

The title compound (E116) was prepared in 97% yield according to a similar procedure to E110 starting from 6-{[4-(6-fluoro-2-methyl-5-quinolinyl)-1-piperazinyl]acetyl}-2H-1,4-benzoxazin-3(4H)-one (E115).

MS: (ES/+) m/z: 437 [MH⁺]. C₂₄H₂₅FN₄O₃ requires 436.

¹H-NMR (500 MHz, d⁶-DMSO) δ(ppm): 10.69 (s, 1H), 8.48 (d, 1H), 7.72 (dd, 1H), 7.55 (dd, 1H), 7.45 (d, 1H) 6.95 (s, 1H) 6.90 (m, 2H), 5.05 (d, 1H), 4.68 (m, 1H), 4.53 (s, 2H), 3.4-2.8 (bs, 4H), 2.63 (s, 3H), 2.6-2.4 (m, 2H).

### Example E117

### 6-{1-Hydroxy-2-[4-(2-methyl-5-quinolinyl)-1-piperazinyl]ethyl}-4-methyl-2H-1,4-benzoxazin-3(4H)-one (E117)

The title compound (E117) was prepared in 53% yield according to a similar procedure to E110 starting from 4-methyl-6-{[4-(2-methyl-5-quinolinyl)-1-piperazinyl]acetyl}-3,4-dihydro-2H-1,4-benzoxazin-2-one (E112).

MS: (ES/+) m/z: 433 [MH⁺]. C₂₅H₂₈N₄O₃ requires 432.

¹H-NMR (300 MHz, CDCl₃) δ(ppm): 8.35 (d, 1H), 7.73 (d, 1H), 7.58 (t, 1H), 7.25 (d, 1H), 7.08 (m, 2H), 6.95 (m, 2H), 4.8 (dd, 1H), 4.6 (s, 2H), 3.4 (s, 3H), 2.7 (s, 3H), 3.3-2.4 (bm, 10H).

### Example E118

### 6-{[4-(8-Fluoro-2-methyl-5-quinolinyl)-1-piperazinyl]acetyl}-4-methyl-2H-1,4-benzoxazin-3(4H)-one (E118)

The title compound (E118) was prepared in 29% yield according to a similar procedure to E112 starting from 8-fluoro-2-methyl-5-piperazin-1-yl-quinoline (D97) and 6-(chloroacetyl)-4-methyl-2H-1,4-benzoxazin-3(4H)-one (D94).

MS: (ES/+) m/z: 449 [MH⁺]. C₂₅H₂₅FN₄O₃ requires 448.

### Example E119

### 6-{2-[4-(8-Fluoro-2-methyl-5-quinolinyl)-1-piperazinyl]-1-hydroxyethyl}-4-methyl-2H-1,4-benzoxazin-3(4H)-one (E119)

The title compound (E119) was prepared in 99% yield according to a similar procedure to E110 starting from 6-{[4-(8-fluoro-2-methyl-5-quinolinyl)-1-piperazinyl]acetyl}-4-methyl-2H-1,4-benzoxazin-3(4H)-one (E118).

MS: (ES/+) m/z: 451 [MH⁺]. C₂₅H₂₇FN₄O₃ requires 450.

¹H-NMR (300 MHz, CDCl₃) δ(ppm): 8.35 (dd, 1H), 7.3 (d, 1H), 7.3 (dd, 1H), 7.07 (bs, 1H), 7.0 (dd, 1H), 6.95 (bs, 2H), 4.80 (dd, 1H), 4.6 (s, 2H), 3.35 (s, 3H), 2.75 (s, 3H), 3.25-2.0 (bm, 10H).

### Example E120

### 6-{[4-(6-Fluoro-2-methyl-5-quinolinyl)-1-piperazinyl]acetyl}-4-methyl-2H-1,4-benzoxazin-3(4H)-one (E120)

The title compound (E120) was prepared in 37% yield according to a similar procedure to E112 starting from 6-fluoro-2-methyl-5-(1-piperazinyl)quinoline (D100) and 6-(chloroacetyl)-4-methyl-2H-1,4-benzoxazin-3(4H)-one (D94).

MS: (ES/+) m/z: 449 [MH⁺]. C₂₅H₂₅FN₄O₃ requires 448.

¹H-NMR (300 MHz, CDCl₃) δ(ppm): 8.52 (d, 1H), 7.80 (m, 3H), 7.37 (m, 1H), 7.28 (t, 1H), 7.03 (d, 1H), 4.67 (s, 2H), 3.84 (s, 2H), 3.70-2.55 (bm, 8H), 3.42 (s, 3H) 2.70 (s, 3H).

### Example E121

### 6-{2-[4-(6-Fluoro-2-methyl-5-quinolinyl)-1-piperazinyl]-1-hydroxyethyl}-4-methyl-2H-1,4-benzoxazin-3(4H)-one (E121)

The title compound (E121) was prepared in 35% yield according to a similar procedure to E110 starting from 6-{[4-(6-fluoro-2-methyl-5-quinolinyl)-1-piperazinyl]acetyl}-4-methyl-2H-1,4-benzoxazin-3(4H)-one (E120).

MS: (ES/+) m/z: 451 [MH⁺]. C₂₅H₂₇FN₄O₃ requires 450.

¹H-NMR (300 MHz, CDCl₃) δ(ppm): 8.5 (d, 1H), 7.8 (dd, 1H), 7.4 (dd, 1H), 7.3 (d, 1H), 7.1 (bs, 1H), 6.95 (m, 2H), 4.75 (dd, 1H), 4.6 (bs, 2H), 3.35 (s, 3H), 2.7 (s, 3H), 3.6-2.3 (bm, 10H).

### Example E122

### 4-Methyl-6-{2-[4-(2-methyl-5-quinolinyl)hexahydro-1H-1,4-diazepin-1-yl]ethyl}-2H-1,4-benzoxazin-3(4H)-one dihydrochloride (E122)

The title compound (E122) was prepared in 44% yield according to the general alkylation procedure starting from 5-[1,4]-diazepan-1-yl-2-methylquinoline (D21) and 6-(2-chloroethyl)-4-methyl-2H-1,4-benzoxazin-3(4H)-one (D43).

MS: (ES/+) m/z: 431 [MH⁺]. C₂₆H₃₀N₄O₂ requires 430.

¹H-NMR (400 MHz, d⁶-DMSO) δ(ppm): 10.89 (bs, 1H), 8.98 (bs, 1H), 7.88 (bs, 2H), 7.79 (bs, 1H), 7.45 (bs, 1H), 7.14 (d, 1H), 6.96 (m, 2H), 4.63 (s, 2H), 3.71 (m, 4H), 3.60 (m, 2H), 3.42 (m, 2H), 3.38 (m, 2H), 3.29 (s, 3H), 3.11 (m, 2H), 2.88 (bs, 3H), 2.32 (m, 1H), 2.23 (bm, 1H).

### Example E123

### 4-Methyl-6-{2-[3-methyl-4-(2-methyl-5-quinolinyl)-1-piperazinyl]ethyl}-2H-1,4-benzoxazin-3(4H)-one (E123)

The title compound (E123) was prepared in 42% yield according to a similar procedure to E11 starting from 6-{2-[3-methyl-4-(2-methyl-5-quinolinyl)-1-piperazinyl]ethyl}-2H-1,4-benzoxazin-3(4H)-one (E15) and methyl iodide.

MS: (ES/+) m/z: 431 [MH⁺]. C₂₆H₃₀N₄O₂ requires 430.

¹H-NMR (300 MHz, CDCl₃) δ(ppm): 8.55 (d, 1H), 7.8 (d, 1H), 7.6 (t, 1H), 7.25 (d, 1H), 6.85 (m, 3H), 4.60 (s, 2H), 3.35 (s, 3H), 2.70 (s, 3H), 3.4 (m, 1H), 3.15-2.75 (m, 6H), 2.65 (m, 2H), 2.55 (m, 1H), 2.25 (m, 1H), 0.85 (d, 3H).

### Example 124

### 6-{2-[4-(8-Chloro-2-methylquinolin-5-yl)-piperazine-1-yl]-ethyl}-4-methyl-4H-benzo[1,4]oxazine-3-one (E124)

A mixture of 8-chloro-2-methyl-5-piperazin-1-ylquinoline (D64) (25 mg, 0.095 mmol), 6-(2-chloroethyl)-4-methyl-4H-benzo[1,4]oxazin-3-one (D43) (30 mg, 0.13 mmol), sodium iodide (20 mg, 0.13 mmol), and sodium carbonate (14 mg, 0.13 mmol) were suspended in N-methylpyrrolidinone (1 ml). The reaction mixture was heated at 120° for 5 hours, cooled to r.t., diluted with ethyl acetate (10 ml), filtered and concentrated to dryness. The oily residue was purified by SPE silica cartridge, eluting with a 99/1 mixture of DCM/MeOH to afford the title compound (E124) (20 mg, 46% yield), which was converted to the corresponding hydrochloride salt using a 1M solution of HCl in diethyl ether.

¹H-NMR (400 MHz, d₆-DMSO) δ(ppm): 10.48 (bs, 1H), 8.46 (d, 1H), 7.83 (d, 1H), 7.54 (d, 1H), 7.19 (d, 1H), 7.12 (d, 1H), 7.00 (d, 1H), 6.96 (dd, 1H), 4.63 (s, 2H), 3.68 (m, 4H), 3.45 (m, 4H), 3.3 (s, 3H), 3.21 (t, 2H), 3.08 (m, 2H), 2.71 (s, 3H).

### Example 125

### 6-{2-[4-(8-Fluoro-2-methyl-quinolin-5-yl)-piperazine-1-yl]-ethyl}-4-methyl-4H-benzo[1,4]oxazine-3-one (E125)

A mixture of 8-fluoro-2-methyl-5-piperazin-1-yl-quinoline (D97) (25 mg, 0.10 mmol), 6-(2-chloroethyl)-4-methyl-4H-benzo[1,4]oxazin-3-one (D43) (30 mg, 0.13 mmol), sodium iodide (20 mg, 0.13 mmol), and sodium carbonate (14 mg, 0.13 mmol) were suspended in N-methylpyrrolidinone (1 ml). The reaction mixture was heated at 120° for 6 hours, cooled to r.t., diluted with ethyl acetate (10 ml), filtered and concentrated to dryness. The oily residue was purified by SPE silica cartridge, eluting with a 98/2 mixture of DCM/MeOH to afford the title compound (E125) (15 mg, 35% yield), which was converted to the corresponding hydrochloride salt using a 1M solution of HCl in diethyl ether.

¹H-NMR (400 MHz, d₆-DMSO) δ(ppm): 10.43 (bs, 1H), 8.45 (d, 1H), 7.54 (d, 1H), 7.48 (dd, 1H), 7.18 (dd, 1H), 7.12 (d, 1H), 6.99 (d, 1H), 6.95 (dd, 1H), 4.63 (s, 2H), 3.7-3.19 (m, 10H), 3.30 (s, 3H), 3.08 (dd, 2H), 2.69 (s, 3H).

### Example 126

### 6-{2-[4-(2-Methyl-1H-indol-4-yl)piperazin-1-yl]ethanoyl}-4H-benzo[1,4]oxazin-3-one (E126)

To a suspension of 2-methyl-5-piperazin-1-ylindole (0.27 g, 1.25 mmol, 1.0 eq) and 6-(chloroacetyl)-2H-1,4-benzoxazin-3(4H)-one (0.4 g, 1.75 mmol, 1.4 eq.) in dry acetonitrile (10 ml) N,N-diisopropylethylamine (0.435 ml, 2.5 mmol, 2 eq) was added. The reaction mixture was stirred at reflux for 4 h, then allowed to cool to r.t. and concentrated in vacuo. The residue was dissolved in water (25 ml) and the mixture extracted with ethyl acetate (2×30 ml). The organic phases were separated, dried (Na₂SO₄) and concentrated in vacuo. The crude product was then purified by flash chromatography, eluting with ethylacetate/cyclohexane 70:30, to afford the title compound (E126) as a pale yellow solid (0.35 g, 69%).

MS; (ES) m/z: 405 [MH⁺]. C₂₃H₂₄N₄O₃ requires 404.

¹H-NMR (300 MHz, DMSO) δ: 10.91 (bs, 1H), 10.78 (bs, 1H), 7.75 (d, 1H); 7.59 (d, 1H); 7.1 (t, 1H); 6.89 (m, 2H); 6.45 (d, 1H); 6.03 (d, 1H), 4.60 (s, 2H), 3.8 (m, 2H), 3.12 (m, 4H), 2.75 (m, 4H), 2.4 (s, 3H)

### Example 127

### 6-{1-Hydroxy-2-[4-(2-methyl-1H-indol-4-yl)piperazinyl]ethyl}-2H-benzo[1.4]oxazin-3-one (E127)

A stirred suspension of 6-{2-[4-(2-methyl-1H-indol-4-yl)piperazin-1-yl]ethanoyl}-4H-benzo[1.4]oxazin-3-one (E126) (0.1 g, 0.25 mmol, 1.0 eq.) in dry methanol (10 ml) was cooled to 0° and sodium borohydride (0.038 g, 1 mmol, 4.0 eq) was added portionwise. The reaction mixture was stirred at 0° under nitrogen for 3 h. The reaction was quenched at r.t. with saturated aq. ammonium chloride and extracted into ethyl acetate. The organic layers were combined, dried (Na₂SO₄) and concentrated in vacuo. The crude product was purified by flash chromatography, eluting with 2% methanol in DCM, to give the title compound (E127) as a white solid (0.03 g, 30%).

MS; (ES) m/z: 407 [MH⁺]. C₂₃H₂₆N₄O₃ requires 406.

¹H-NMR (300 MHz, DMSO) δ: 11.0 (bs, 1H), 10.8 (bs, 1H), 7.1 (d, 1H); 6.9-6.82 (m, 4H); 6.45 (d, 1H); 6.03 (d, 1H), 5.1 (d, 1H), 4.7 (m, 1H); 4.58 (s, 2H), 3.10 (m, 4H), 2.72 (m, 4H), 2.4 (s, 3H).

### Example 128

### 6-{1-Fluoro-2-[4-(2-methyl-1H-indol-4-yl)piperazinyl]ethyl}-2H-benzo[1.4]oxazin-3-one (E128)

A stirred suspension of 6-{1-hydroxy-2-[4-(2-methyl-1H-indol-4-yl)piperazinyl]ethyl}-2H-benzo[1.4]oxazin-3-one (E127) (0.07 g, 0.172 mmol, 1.0 eq.) in dry DCM (5 ml) was cooled to 0° and DAST (0.034 ml, 0.26 mmol, 1.5 eq) was added dropwise. The reaction mixture was stirred at 0° under nitrogen for 3 h, then left at room temperature overnight.

Solvent was evaporated and the crude purified by flash chromatography, eluting with ethylacetate/cyclohexane 1:1, to give the title compound (E128) as a yellow oil (0.01 g, yield 20%).

MS; (ES) m/z: 409 [MH⁺]. C₂₄H₂₅N₄O₃ requires 408.

¹H-NMR (300 MHz, DMSO) δ: 8.7 (bs, 1H), 7.9 (bs, 1H), 7.1 (d, 1H); 7.1-6.9 (m, 3H); 6.85 (d, 1H); 6.65 (m, 1H), 6.2 (d, 1H), 5.75 (m, 1H), 4.58 (s, 2H), 3.10 (m, 4H), 2.72 (m, 4H), 2.4 (s, 3H).

### Example 129 and 130

### 6-{2-[4-(7-Fluoro-2-methyl-5-quinolinyl)-1-piperazinyl]-1-hydroxyethyl}-2-H-1,4-benzoxazin-3-(4H)-one hydrochloride. Separation of enantiomers (E129 and E130)

The two enantiomers of title compound were separated from racemic 6-{2-[4-(7-fluoro-2-methyl-5-quinolinyl)-1-piperazinyl]-1-hydroxyethyl}-2H-1,4-benzoxazin-3(4H)-one (E71) (210 mg, 0.48 mmol) by preparative HPLC (Chiralcel OD 25 cm×20 mm), elution with 15% ethanol in hexane. The resulting fractions were concentrated in vacuo, dissolved in MeOH (5 ml) and treated with 1.25 M HCl in MeOH (0.42 ml, 0.53 mmol, 2.3 eq). The mixtures were stirred at r.t. for 2 h, then concentrated in vacuo and the residue triturated with diethylether (10 ml) at r.t for 1 h.

After filtration, the enantiomerically pure title compounds (E129) (0.101 g) and (E130) (0.0986 g) were obtained as yellow solids (unknown stereochemistry).

Enantiomeric purity was verified by analytical chiral HPLC (Chiralcel OD 25 cm×4.6 mm, elution with 15% ethanol in hexane):

E129, enantiomer 1, Rt=19.3 min
E130, enantiomer 2, Rt=26.5 min

MS; (ES) m/z: 437 [MH⁺]. C₂₄H₂₅N₄O₃ requires 436.

¹H-NMR (500 MHz, DMSO) δ: 10.84 (s, 1H), 10.15 (bs, 1H), 8.37 (d, 1H), 7.40 (m, 2H), 7.13 (d, 1H), 6.9 (s, 1H), 6.97 (s, 1H), 6.3 (bs, 1H), 5.09 (dd, 1H), 4.55 (s, 2H), 3.72 (bt, 2H), 3.6-3.2 (bm, 8H+H₂O), 2.65 (s, 3H).

### Example 131

### 6-{2-[4-(2-Methyl-5quinolinyl)-1-piperadinyl}-ethanoyl}-2H-1,4-benzoxazin-3(4H)-one (E131)

To a suspension of 2-methyl-5-piperidin-4-ylquinoline (D19) (0.11 g, 0.442 mmol, 1.0 eq) and 6-(chloroacetyl)-2H-1,4-benzoxazin-3(4H)-one (0.14 g, 0.62 mmol, 1.4 eq.) in dry acetonitrile (10 ml) N,N-diisopropylethylamine (0.15 ml, 0.8 mmol, 2 eq.) was added. The reaction mixture was stirred at reflux for 4 h, then allowed to cool to r.t. and concentrated in vacuo. The residue was dissolved in water (25 ml) and the mixture extracted with ethyl acetate (2×30 ml). The organic phase was separated, dried (Na₂SO₄) and concentrated in vacuo. The crude product was then purified by flash chromatography, eluting with DCM/MeOH 98:2, to afford the title compound (E131) as a pale yellow solid (0.1 g, 54%).

MS; (ES) m/z: 416 [MH⁺]. C₂₅H₂₅N₃O₃ requires 415

### Example 132

### 6-{1-Hydroxy-2-[4-(2-methyl-5quinolinyl)-1-piperadinyl}-ethyl}-2H-1,4-benzoxazin-3(4H)-one dihydrochloride (E132)

A stirred suspension of 6-{2-[4-(2-methyl-5quinolinyl)-1-piperadinyl}-ethanoyl}-2H-1,4-benzoxazin-3(4H)-one (E131) (0.1 g, 0.241 mmol, 1.0 eq.) in dry MeOH (10 ml) was cooled to 0° and sodium borohydride (0.038 g, 1 mmol, 4.0 eq) was added portionwise. The reaction mixture was stirred at 0° under nitrogen for 3 h. 1M HCl acid solution was added and the resulting mixture was passed trough a SCX column and then, after evaporation of the ammonia fractions, the crude was purified by flash chromatography, eluting with 2% methanol in DCM, to give the free base of the title compound (E132) as a white solid (0.052 mg, 30%).

The free base was then dissolved in MeOH (2 ml) and HCl in MeOH (0.2 ml of a 1.25M solution) was added.

After filtration, 0.036 g of the corresponding dihydrochloride salt was isolated as yellow solid.

MS; (ES) m/z: 418 [MH⁺]. C₂₅H₂₇N₃O₃ requires 417.

¹H-NMR (300 MHz, DMSO) δ: 11.0 (bs, 1H), 10.5 (bs, 1H), 8.8 (bs, 1H), 7.9 (t, 1H), 7.75 (m, 1H), 7.58 (d, 1H), 7.39 (d, 1H), 7.00 (m, 3H), 6.3 (d, 1H), 5.2 (m, 1H), 4.7 (m, 1H); 4.58 (s, 2H), 3.8-3.6 (m, 3H), 3.4-3.2 (m, 4H), 2.70 (s, 3H), 1.90-2.15 (m, 4H).

### Example 133

### 6-{2-[4-(7-Fluoro-2-methylquinolin-5-yl)piperidin-1-yl]ethyl}-4-H-benzo[1,4]-oxazin-3-one dihydrochloride salt (E133)

The title compound (E133) was prepared from 7-fluoro-2-methylquinolin-5-(1,2,3,6-tetrahydropyridin-4-yl]-quinoline (D104) and 6-(2-chloro)ethyl-4H-benzo[1,4]-oxazin-3-one (D4) according to the general procedure in 31% yield.

MS; (ES) m/z: 420.2 [MH⁺]. C₂₅H₂₆FN₃O₂ requires 419.

¹H-NMR (300 MHz, DMSO) δ: 10.9 (s, 1H), 9.45 (br s, 1H), 8.6 (d, 1H), 7.65 (m, 1H), 7.50 (d, 1H), 7.35 (m, 1H), 6.95-6.7 (m, 3H), 4.55 (s, 2H), 3.8 (m, 2H), 3.2-3.55 (m, 5H, +H₂O), 3.1 (m, 2H), 2.70 (s, 3H), 1.90-2.15 (m, 4H).

### Example 134 and 135

### 6-{2-[4-(6-Fluoro-2-methyl-5-quinolinyl)-1-piperazinyl]-1-hydroxyethyl}-2H-1,4-benzoxazin-3(4H)-one. Separation of Enantiomers (E134 and E135)

The title compounds were separated from racemic 6-{2-[4-(6-fluoro-2-methyl-5-quinolinyl)-1-piperazinyl]1-hydroxyethyl}-2H-1,4-benzoxazin-3-(4H)-one (E116) (28 mg, 0.064 mmol) by preparative HPLC (Chiralcel OJ 25 cm×20 mm), elution with 40% ethanol in hexane. After evaporation of the appropriate fractions the resulting pure enantiomers were dissolved in methanol (2 ml) and treated with 1.25 M HCl in MeOH (2.3 eq) to afford the title compounds (E134) (0.011 g) and (E135) (0.011 g) as yellow solids with unknown stereochemistry.

Enantiomeric purity was verified by analytical chiral HPLC (Chiralcel OJ 25 cm×4.6 mm, elution with 15% ethanol in hexane):

### E134—Rt=13.4 min

### E135—Rt=19.7 min

MS; (ES) m/z: 437 [MH⁺]. C₂₄H₂₅FN₄O₃ requires 436.

¹H-NMR (500 MHz, DMSO) δ: 10.88 (s, 1H), 10.06 (bs, 1H), 8.72 (bd, 1H), 7.94 (bm, 1H), 7.75 (t, 1H), 7.66 (d, 1H), 7.02 (m, 3H), 6.31 (bs, 1H), 5.12 (dd, 1H), 4.59 (s, 2H), 3.8-3.5 (bm, 6H), 3.32 (bm, 4H), 2.76 (bs, 3H).

### Example 136

### 6{2-[4(8-Fluoro-2-methyl-5-quinolinyl)-1-piperazinyl]ethyl}-2H-1,4-benzoxazin-3-(4H)-one (E136)

The title compound (E136) was prepared from 8-fluoro-2-methyl-5-piperidin-4-ylquinoline (WO02/34754) and 6-(2-chloro)ethyl-4H-benzo[1,4]oxazin-3-one (D4) according to the general procedure in 42% yield.

MS; (ES) m/z: 421.2 [MH⁺]. C₂₅H₂₆FN₄O₂ requires 420.

¹H-NMR (500 MHz, DMSO) δ: 10.65 (s, 1H), 8.39 (d, 1H), 7.50 (d, 1H), 7.42 (q, 1H), 7.09 (q, 1H), 6.86 (d, 1H), 6.78 (m, 2H), 4.51 (s, 2H), 2.99 (m, 4H), 2.7 (m, 6H), 2.66 (s, 3H), 2.56 (bm, 2H).

### Example 137

### 6-{2-[4-2-Quinoxalinyl)-1-piperazinyl]ethyl}-2H-1,4-benzoxazin-3-(4H)-one (E137)

The title compound (E137) was prepared starting from 2-(1-piperazinyl)quinoxaline (0.07 g, 0.325 mmol) and 6-(2-chloro)ethyl-4H-benzo[1,4]oxazin-3-one (D4) according to the general procedure described above in 47% yield.

MS; (ES) m/z: 390.3 [MH⁺]. C₂₂H₂₃N₅O₂ requires 389.

¹H-NMR (300 MHz, DMSO) δ: 10.95 (s, 1H), 9.8 (bs, 1H), 8.9 (d, 1H), 7.95 (d, 1H), 7.52 (t, 1H), 6.8 (m, 2H), 4.51 (s, 2H), 2.99 (m, 4H), 2.7 (m, 6H), 2.66 (s, 3H), 2.56 (bm, 2H).

### Example 138

### 4-Methyl-8-{2-[(2R)-2-methyl-4-(2-methyl-5-quinolinyl)-1-piperazinyl]ethyl}-2H-1,4-benzoxazin-3(4H)-one hydrochloride salt (E138)

A mixture of (4-methyl-3-oxo-3,4-dihydro-2H-1,4-benzoxazin-8-yl)acetaldehyde (D69) (27.4 mg, 0.13 mmol) and 2-methyl-5-[(3R)-3-methyl-1-piperazinyl]quinoline (D71) (48.2 mg, 0.20 mmol) in dry 1,2-dichloroethane (3 ml) was stirred at room temperature under nitrogen for 40 min. Sodium triacetoxyborohydride (42.4 mg, 0.20 mmol) was then added and the resulting reaction mixture was stirred for 4 h, quenched with a saturated aqueous solution of NaHCO₃ (5 ml) and extracted with DCM (3×10 ml). The organic layers were combined, dried (Na₂SO₄) and concentrated in vacuo. The crude product was purified by SPE cartridge (Si), eluting with 2% methanol in DCM to afford the corresponding free base of the title compound (E138) as a solid (20.4 mg, 36%).

MS; (ES) m/z: 431.40 [MH⁺]. C₂₆H₃₀N₄O₂ requires 430.55.

¹H-NMR (300 MHz, DMSO-d⁶) δ: 8.39 (d, 1H), 7.59 (m, 2H), 7.34 (d, 1H), 7.09 (d, 1H), 7.05-6.92 (m, 3H), 4.64 (s, 2H), 3.22 (s, 3H), 3.19-2.55 (m, 11H), 2.67 (s, 3H), 1.11 (d, 3H).

The free base was dissolved in dry methanol (3 ml) and a 1.25 M solution of hydrochloric acid in methanol (0.062 ml, 0.12 mmol) was slowly added at 0° C. The resulting suspension was stirred at 0° C. for 4 h. Evaporation of the volatile gave the title compound (E138) as a solid (23.1 mg, 98%).

### Example 139

### 4-Methyl-8-{2-[(2S)-2-methyl-4-(2-methyl-5-quinolinyl)-1-piperazinyl]ethyl}-2H-1,4-benzoxazin-3(4H)-one (E139)

The corresponding free base of the title compound (E139) was prepared as a solid in 39% yield in a similar fashion to Example 138 starting from (4-methyl-3-oxo-3,4-dihydro-2H-1,4-benzoxazin-8-yl)acetaldehyde (D69) (17.4 mg, 0.085 mmol) and 2-methyl-5-[(3S)-3-methyl-1-piperazinyl]quinoline (D72) (30.8 mg, 0.13 mmol).

MS; (ES) m/z: 431.40 [MH⁺]. C₂₆H₃₀N₄O₂ requires 430.55.

¹H-NMR (300 MHz, DMSO-d⁶) δ: 8.39 (d, 1H), 7.59 (m, 2H), 7.34 (d, 1H), 7.09 (d, 1H), 7.05-6.92 (m, 3H), 4.64 (s, 2H), 3.22 (s, 3H), 3.19-2.55 (m, 11H), 2.67 (s, 3H), 1.11 (d, 3H).

The free base was dissolved in dry methanol (3 ml) and a 1.25 M solution of hydrochloric acid in methanol (0.095 ml, 0.12 mmol) was slowly added at 0° C. The resulting suspension was stirred at 0° C. for 4 h. Evaporation of the volatile gave the title compound (E139) as a solid (15.6 mg, 94%).

### Examples 140-144

The following Examples (E140-144) were prepared according to the following general procedure:

To a solution of the appropriate substituted 1-(2,3-dihydro-1,4-benzodioxin-5-yl)piperazine hydrochloride (D83, D88, D93 and D108) (0.35 mmol) in methyl isobutyl ketone (MIBK, 8 ml) was added 6-(2-chloroethyl)-4H-benzo[1,4]oxazin-3-one (D4) (1.3 eq) followed by sodium carbonate (3 eq) and sodium iodide (1.3 eq). This mixture was heated to 100° C. for 16 hours. The reaction mixture was purified by SPE extraction using a 5 g SCX cartridge, followed by a general purification on a 5 g silica cartridge. The target compounds were dissolved in Et₂O and a solution of 10% HCl in Et₂O was added at 0° C. The suspension was stirred at room temperature for 1 hour and finally the corresponding hydrochloride salt was filtered. Yields may vary from 30% to 70%.

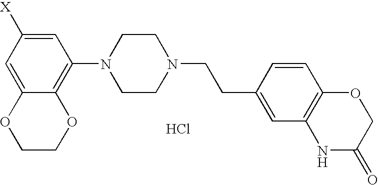

| Example | X | Name | Analytical data |
|---|---|---|---|
| 140 | Cl | 6-{2-[4-(7-Chloro-2,3- | ¹H-NMR (CDCl₃, 343 K, as free base) δ: |
| | | dihydro-1,4- | 7.68 (s br, 1H); 6.90 (d, 1H); 6.82 (dd, |
| | | benzodioxin-5-yl)-1- | 1H); 6.64 (d, 1H); 6.60 (d, 1H); 6.49 (d, |
| | | piperazinyl]ethyl}-2H- | 1H); 4.58 (s, 2H); 4.29 (m, 2H); |
| | | 1,4-benzoxazin-3(4H)-one | 4.23 (m, 2H); 3.09 (m, 4H); 2.76 (m, 2H); |
| | | | 2.69 (m, 4H); 2.62 (m, 2H). |
| | | | ESI POS; AQA: spray 3.5 KV/source |
| | | | 30 V/probe 250° C.: 430.1 (MH+) |
| 141 | F | 6-{2-[4-(7-Fluoro-2,3- | ¹H-NMR (CDCl₃, 343 K, as free base) δ: |
| | | dihydro-1,4- | 7.58 (s br, 1H); 6.90 (d, 1H); 6.82 (dd, |
| | | benzodioxin-5-yl)-1- | 1H); 6.63 (d, 1H); 6.31 (dd, 1H); |
| | | piperazinyl]ethyl}-2H- | 6.27 (dd, 1H); 4.58 (s, 2H); 4.30-4.21 (m, |
| | | 1,4-benzoxazin-3(4H)- | 4H); 3.10 (m, 4H); 2.80-2.58 (m, 8H). |
| | | one | ESI POS; AQA: spray 3.5 KV/source |
| | | | 30 V/probe 250° C.: 414 (MH+) |
| 142 | Br | 6-{2-[4-(7-Bromo-2,3- | ¹H-NMR (CDCl₃, 343 K, as free base) δ: |
| | | dihydro-1,4- | 7.63 (s br, 1H); 6.90 (d, 1H); 6.83 (dd, |
| | | benzodioxin-5-yl)-1- | 1H); 6.74 (d, 1H); 6.64 (d, 1H); 6.62 (d, |
| | | piperazinyl]ethyl}-2H- | 1H); 4.59 (s, 2H); 4.29 (m, 2H); |
| | | 1,4-benzoxazin-3(4H)- | 4.23 (m, 2H); 3.09 (m, 4H); 2.80-2.58 (m, |
| | | one | 8H). ESI POS; AQA: spray 3.5 KV/ |
| | | | source 30 V/probe 250° C.: |
| | | | 474.2 (MH+). |
| 143 | CN | 8-{4-[2-(3-Oxo-3,4- | ESI POS; AQA: spray 3.5 KV/source |
| | | dihydro-2H-1,4- | 30 V/probe 250° C.: 421.24 (MH+) |
| | | benzoxazin-6-yl)ethyl]-1- | |
| | | piperazinyl}-2,3-dihydro- | |
| | | 1,4-benzodioxin-6- | |
| | | carbonitrile | |

1. A compound which is 2-methyl-5-piperazin-1-ylquinoline.

2. A compound which is tert-butyl 4-(2-methylquinolin-5-yl)piperazine-1-carboxylate.
